(19) United States
(12) Patent Application Publication
(10) Pub. No.: US 2025/0170265 A1
(43) Pub. Date: May 29, 2025

(54) DIAZONIUM COMPOUNDS AND METHODS OF USE FOR DNA CLEAVAGE

(71) Applicant: New York University, New York, NY (US)

(72) Inventors: Tianning DIAO, New York, NY (US); Benjamin BEN-ZVI, Long Island City, NY (US); Christina LIAN, Staten Island, NY (US); Maureen BRUSCO, Lakeville, MN (US)

(73) Assignee: New York University, New York, NY (US)

(21) Appl. No.: 18/960,418

(22) Filed: Nov. 26, 2024

Related U.S. Application Data
(60) Provisional application No. 63/603,936, filed on Nov. 29, 2023.

(51) Int. Cl. A61K 47/68; A61K 47/54; A61P 35/00; C07C 69/017; C07C 69/76; C07C 233/75; C07C 275/30; C07D 219/08; C07K 16/28; C07K 16/32

(52) U.S. Cl. CPC A61K 47/6855; A61K 47/549; A61K 47/6849; A61P 35/00; C07C 69/017; C07C 69/76; C07C 233/75; C07C 275/30; C07D 219/08; C07K 16/2803; C07K 16/32

(57) ABSTRACT

The present disclosure relates to aryl diazonium compounds and methods of cleaving DNA using the diazonium compounds as described herein. Further, the present disclosure relates to said aryl diazonium compounds conjugated to additional compounds as described herein.

### CROSS-REFERENCE TO RELATED APPLICATIONS

This patent application claims the benefit of U.S. Provisional Application No. 63/603,936, filed Nov. 29, 2023, the disclosure of which is incorporated by reference herein in its entirety for all purposes.

### STATEMENT REGARDING FEDERALLY SPONSORED RESEARCH OR DEVELOPMENT

This invention was made with government support under R01 GM127778 awarded by the National Institutes of Health. The government has certain rights in the invention.

### FIELD OF THE INVENTION

The present disclosure relates to aryl diazonium compounds and conjugates thereof. Methods of cleaving DNA using the diazonium compounds and conjugates thereof as described herein are disclosed. The present disclosure further relates to methods of treating disease with the compounds and conjugates thereof as described herein.

### BACKGROUND

DNA cleavage, a fundamental biological process involving the disconnection of phosphodiester bonds within the DNA molecule, plays a crucial role in degrading foreign or harmful DNA, such as cancerous, viral, and bacterial DNA. Many therapeutic strategies utilize DNA cleavage as a mechanism of action (Galm, et al., 2005).

Radical reactions are ubiquitous in biological systems, playing pivotal roles in numerous physiological processes (Halliwell, et al., 2015). These reactions proceed through radical intermediates which have unpaired valence electrons that allow them to exhibit high intrinsic reactivity in a range of biological functions, from enzymatic mechanisms to cellular signal transduction. While the high reactivity of radicals can pose challenges due to its potential to damage biomolecules, they also have profound applications in biomedical research, provided one can control their generation under mild, physiologically compatible conditions.

Aryl radicals are among the most reactive radical species, as the unpaired electron occupies an sp 2-hybridized orbital which is not stabilized through hyperconjugation. A natural mechanism to form aryl radicals involves the formation of 1,4-benzenoid diradicals (Abe, et al., 2013) through chemically or thermally activated Bergman cyclization of strained enediynes (Jones, et al., 1972; Nicolaou, et al., 1991) that act as warheads. This process subsequently leads to the abstraction of hydrogen atoms from the C5′-position of 2-deoxy-ribose of DNA, forming 2-deoxyribosyl radicals. Trapping of the radicals by oxygen thereby initiates unrepairable double-strand DNA backbone cleavage followed by cell-cycle arrest and apoptotic cell death (Lee, et al., 1991; Zein, et al., 1988; Hangeland, et al., 1992; Myers, et al., 1994).

The structural feature of strained enediynes is shared amongst several natural products including calicheamicin γ₁, esperamicin A, dynemicin A, neocarzinostatin, and shishijimicin A (Zein, et al., 1988). These natural products exhibit excellent potency in double-stranded DNA cleavage with half maximal effective concentrations (EC₅₀) ranging from 14-20 nM (Lee, et al., 1991). Calicheamicin γ₁ is renowned for its remarkable potency in DNA cleavage, which is considered the most potent molecule for several applications, including inducing cancer cell death (Lee, Dunne, et al., 1992). Calicheamicin γ₁ has been successfully utilized in FDA-approved drugs for leukemia treatment, whose efficacy is attributed to the DNA-cleaving activity of the calicheamicin γ₁ component. Calicheamicin γ₁ serves as the active cytotoxic element in FDA-approved antitumor drugs Gemtuzumab ozogamicin (Mylotarg™) and Inotuzumab ozogamicin (Besponsa™), enabled by the creation of antibody-drug conjugates (ADCs) through the conjugation of calicheamicin γ₁ to differing antibodies (Tong, et al., 2021; Hamann, et al., 2002; Damle, et al., 2003).

However, the intricate structure of calicheamicins and related natural products presents significant challenges for synthesis, primarily due to the instability of the strained enediyne structures and the complexity of the molecular scaffolds (Kishikawa, et al., 1991; Zhang, et al., 2019; Nicolaou, et al., 1991; Lee, Dunne, et al., 1992; Galm, et al., 2005; Basak, et al., 2003). At present, the commercial production of calicheamicin γ₁ relies on the isolation of the core structure from bacteria cultures, a process that necessitates rigorous and expensive purification and quality control measures (Hamann, et al., 2002; Lee, Greenstein, et al., 1992; Chen, et al., 2022), and the total synthesis of calicheamicin γ₁ spans 36 steps (Smith, et al., 1992; Nicolaou, Hummel, et al., 1992). These synthetic challenges result in high production costs and challenges to derivatize the natural products to adjust their potency and efficacy for tuning the therapeutic window. The modularity of payload toxicity is crucial for developing a successful ADC, as the toxicity should be within an optimal range rather than maximized (Nguyen, et al., 2023). The lack of tunability in the toxicity of calicheamicin γ₁ may have contributed to its severe side effects (McDonald, et al., 2019).

A small molecule mimic of calicheamicin γ₁, capable of generating aryl radicals, could address challenges related to production costs and tunability. Currently, there is a scarcity of small molecule mimetics of calicheamicin γ₁ and other potent DNA-cleaving natural products. While Nicolaou's strained enediyne mimic exhibits high cytotoxicity (Nicolaou, Dai, et al., 1992), the simpler analogues are much less potent in DNA cleavage assays (Nicolaou, et al., 1988; Arya, et al., 1993; Arya, et al., 1995; Kizil, et al., 2003; Mohler, et al., 2003; Ylijoki, et al., 2012; Bhattacharya, et al., 2018). Many synthetic mimetics display a low potency, with EC₅₀ values as high as millimolar (mM) to low micromolar (μM) range. These values are considerably higher compared to the low nanomolar (nM) concentrations enabled by calicheamicin γ₁ and are not suitable for pursuing as drug candidates. The synthesis of many of these mimetics is arduous and necessitates numerous synthetic steps. Some agents require the involvement of transition metal reductants or catalysts for activation, further limiting their potential as drug candidates (Arya, 2006). Often, elevated temperatures, extended reaction times, and/or nonoptimal conditions, such as acidic solvents, may be needed.

Accordingly, there is an unmet need in the art for synthetically accessible and easily derivatized small molecules with improved potency for DNA cleavage. Additionally, there remains a need for controlled generation of aryl radicals under mild, physiologically compatible conditions to address long-standing challenges in DNA cleavage.

### SUMMARY OF THE INVENTION

In one aspect, provided herein is a compound having the structure according to Formula (I):

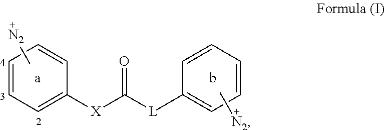

or a pharmaceutically acceptable salt thereof, wherein: X is selected from the group consisting of S, Se, P, NH, and O; and L is selected from the group consisting of S, Se, P, bond and NH; wherein: when X is NH and L is bond, N₂⁺ in ring a is not located at the 4 position. In certain embodiments, the compound is:

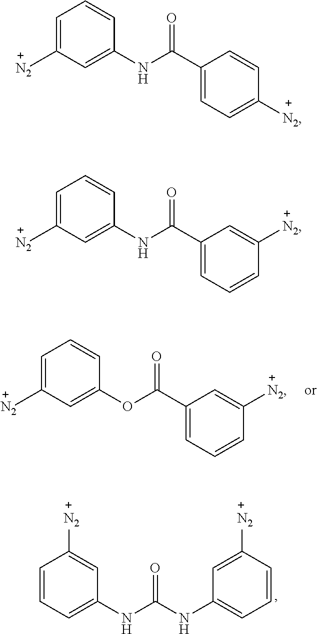

or a pharmaceutically acceptable salt thereof.

In a further aspect, provided herein is a compound having the structure according to Formula (II):

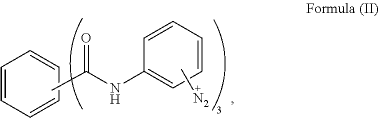

or a pharmaceutically acceptable salt thereof.

In certain embodiments, the compound is

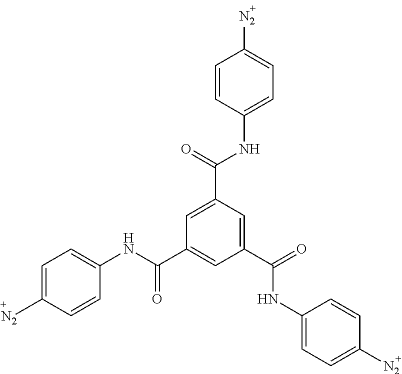

or a pharmaceutically acceptable salt thereof.

In another aspect, provided herein is a compound having the structure selected from the group consisting of Formula (III) and Formula (IV):

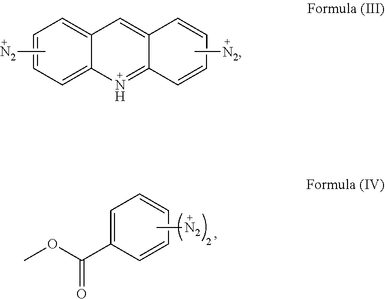

or a pharmaceutically acceptable salt thereof. In certain embodiments, the compound is:

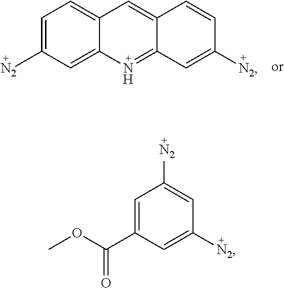

or a pharmaceutically acceptable salt thereof.

In another aspect, provided herein is a compound having the structure according to Formula (V):

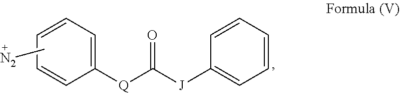

or a pharmaceutically acceptable salt thereof wherein: Q is selected from the group consisting of NH and bond; and J is selected from the group consisting of NH and bond. In certain embodiments, the compound is:

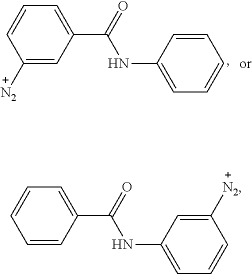

or a pharmaceutically acceptable salt thereof.

In a further aspect, provided herein is a compound having the structure according to Formula (XVII):

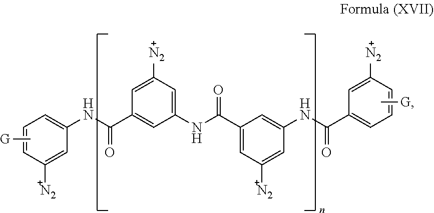

or a pharmaceutically acceptable salt thereof, wherein: G is independently at each occurrence selected from the group consisting of H, halogen, carbonyl, ester, ketone, amide, alkyl, cycloalkyl, aryl, arylalkyl, alkenyl, heteroalkyl, cyano, alkoxy, amine, haloalkyl, heteroatoms, and heterocycle; and n is selected from the group consisting of 1, 2, 3, 4, 5, 6, 7, 8, 9, and 10. In certain embodiments, the compound is

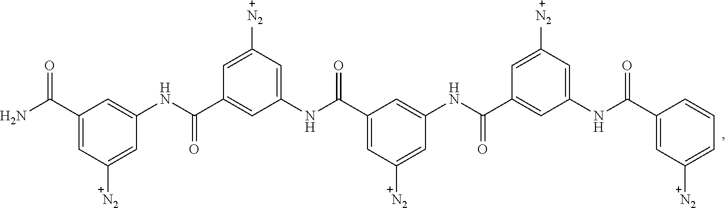

or a pharmaceutically acceptable salt thereof.

In another aspect, provided herein is a compound having the structure selected from the group consisting of Formula (XVIII), Formula (XIX), Formula (XX), Formula (XXI), Formula (XXII), and Formula (XXIII):

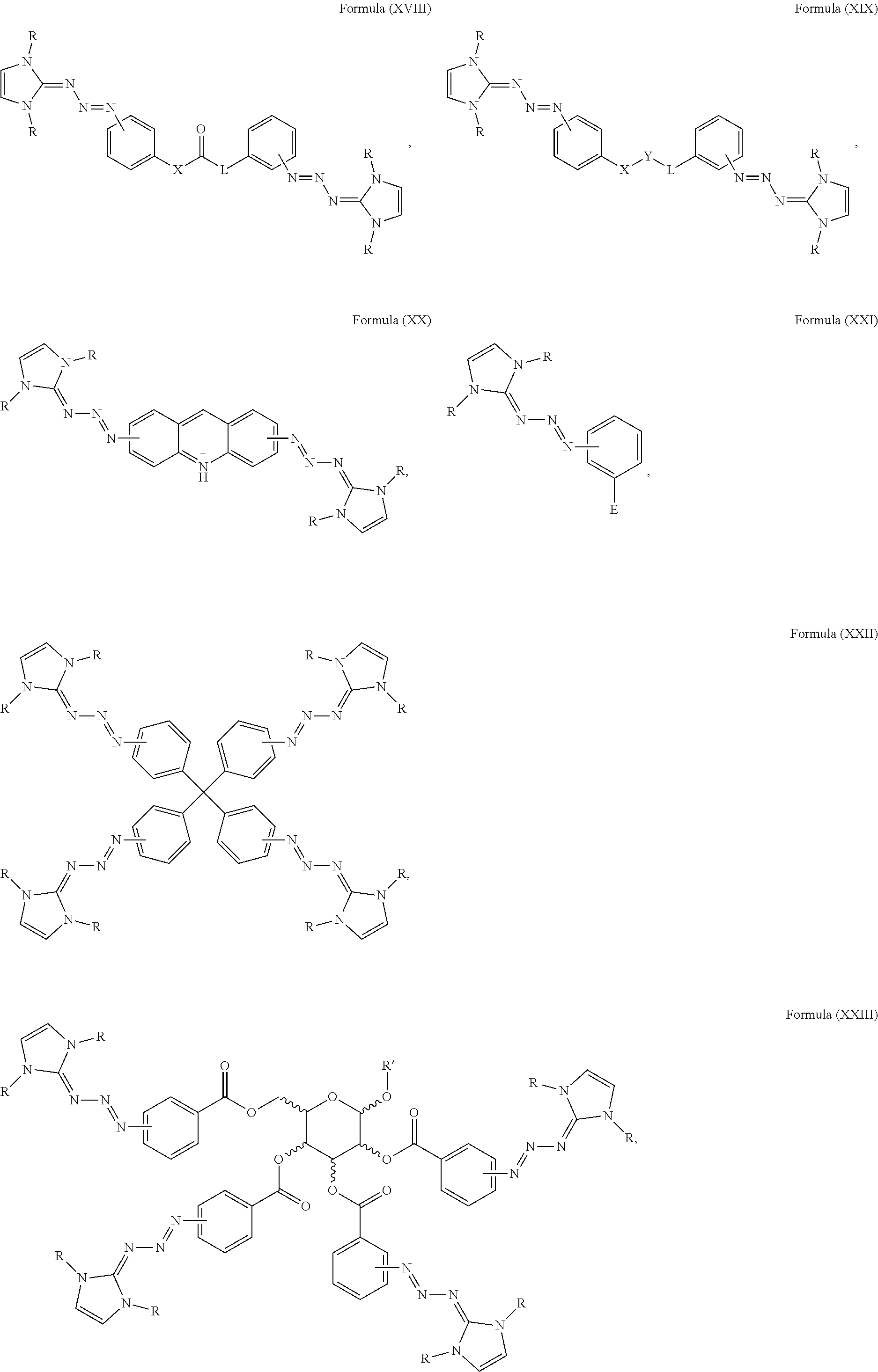

or a pharmaceutically acceptable salt thereof, wherein: R is selected from the group consisting of methyl, ethyl, isopropyl, mesityl, and alkyl sulfonate; X is selected from the group consisting of S, Se, P, bond, NH, NMe, and O; L is selected from the group consisting of S, Se, P, bond, and NH; Y is selected from the group consisting of O and C═O; E is selected from the group consisting of CN, CO₂Me, OMe, and NO₂; and R′ is selected from the group consisting of H, alkyl, cycloalkyl, halogen, alkenyl, aryl, arylalkyl, haloalkyl, heteroalkyl, acetyl, heterocycle, amine, amide, ether, and carbonyl, and any combination thereof.

In a further aspect, provided herein is a compound having the structure selected from the group consisting of Formula (XXV), Formula (XXVI), Formula (XXVII), Formula (XXVIII), Formula (XXIX), Formula (XXX), and Formula (XXXI):

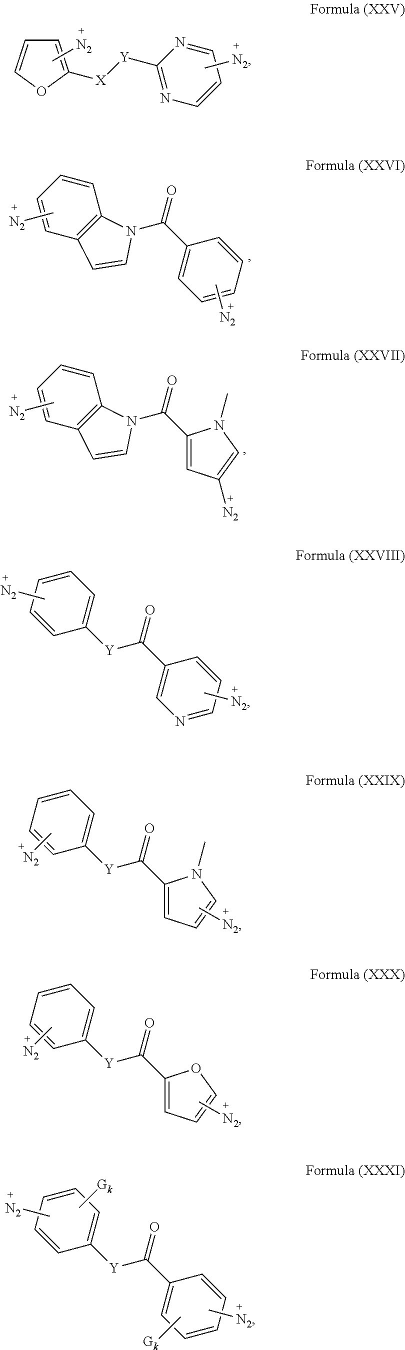

or a pharmaceutically acceptable salt thereof, wherein: X and Y are independently selected from the group consisting of bond, alkyl, S, Se, P, C═O, NH, NR′, S, O, amide, and ester; k is independently at each occurrence from 0 to 4; G is independently at each occurrence selected from the group consisting of H, halogen, carbonyl, ester, ketone, amide, alkyl, cycloalkyl, aryl, arylalkyl, alkenyl, heteroalkyl, cyano, alkoxy, amine, haloalkyl, heteroatoms, and heterocycle; and R′ is selected from the group consisting of H, alkyl, cycloalkyl, halogen, alkenyl, aryl, arylalkyl, haloalkyl, heteroalkyl, acetyl, heterocycle, amine, amide, ether, and carbonyl, and any combination thereof.

In another aspect, provided herein is a compound having the structure selected from the group consisting of Formula (I), Formula (II), Formula (III), Formula (IV), Formula (V), Formula (VI), Formula (VII), Formula (VIII), Formula (IX), Formula (X), Formula (XI), Formula (XII), Formula (XIII), Formula (XIV), Formula (XVII), Formula (XVIII), Formula (XIX), Formula (XX), Formula (XXI), Formula (XXII), Formula (XXIII), Formula (XXIV), Formula (XXV), Formula (XXVI), Formula (XXVII), Formula (XXVIII), Formula (XXIX), Formula (XXX), Formula (XXXI), and Formula (XXXII):

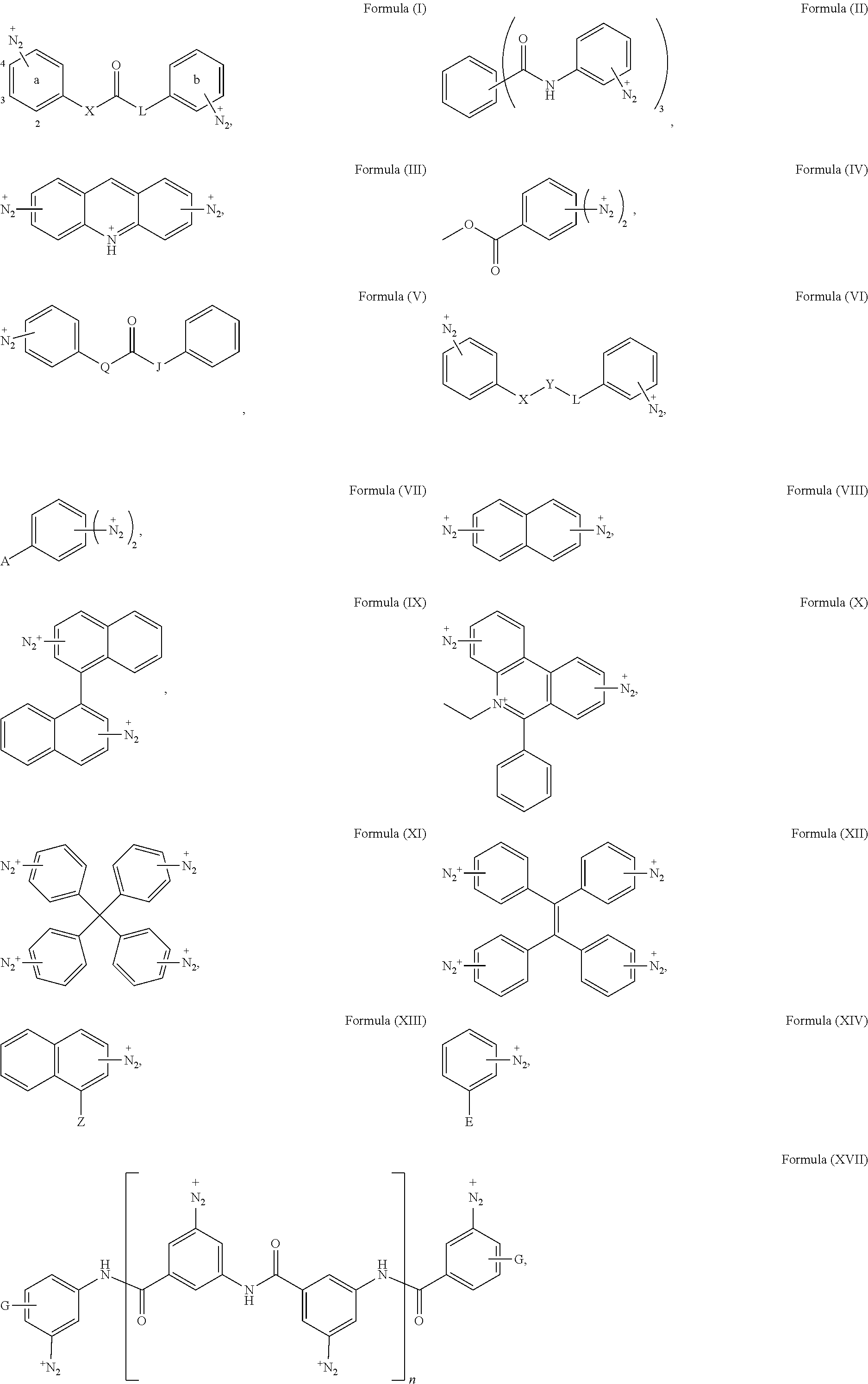

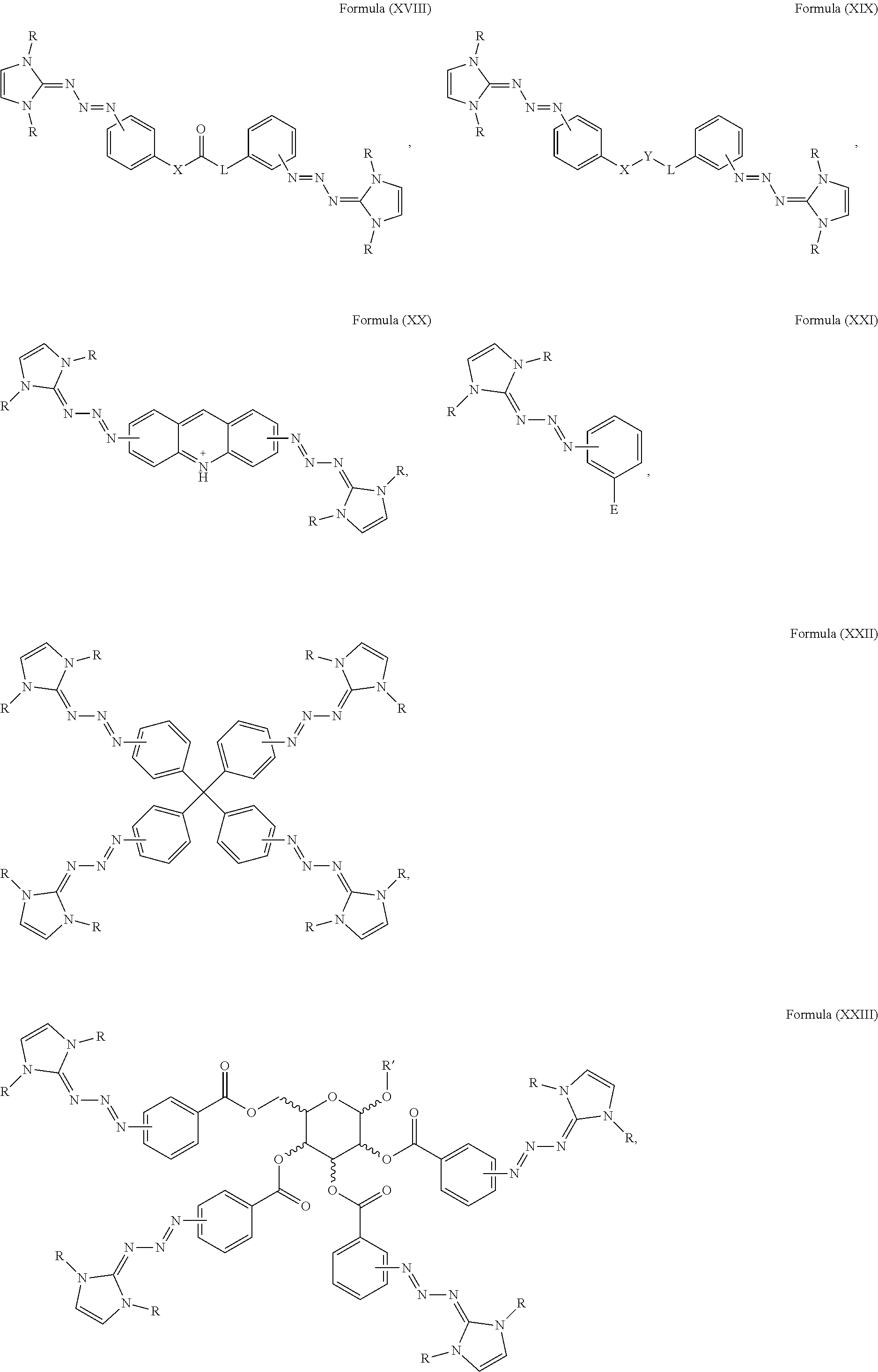

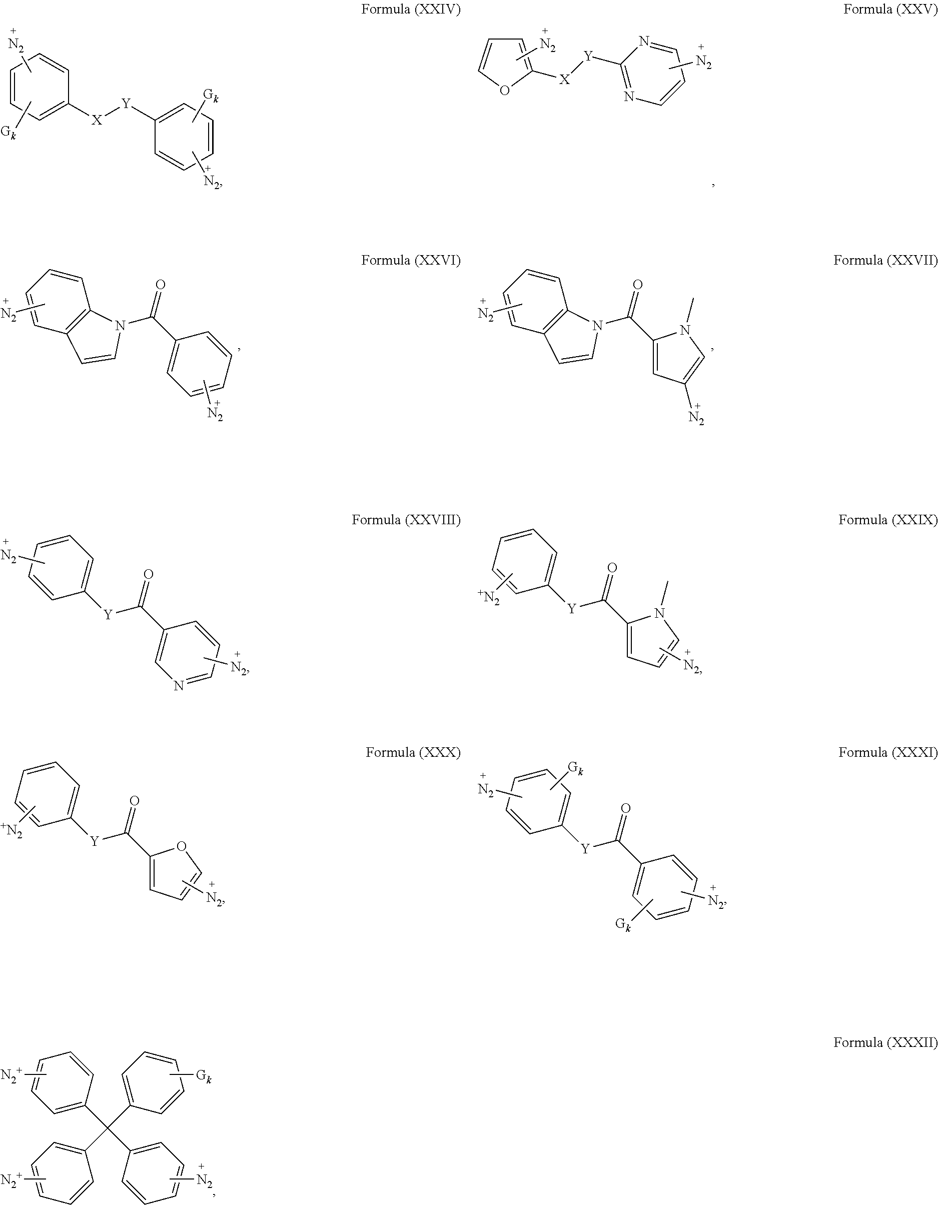

or a pharmaceutically acceptable salt thereof, wherein: X is selected from the group consisting of S, Se, P, bond, alkyl, C═O, NH, NR′, amide, ester, and O; L is selected from the group consisting of S, Se, P, bond, and NH; Q is selected from the group consisting of NH and bond; J is selected from the group consisting of NH and bond; Y is selected from the group consisting of bond, alkyl, S, Se, P, NH, NR′, S, amide, ester, 0, and C═O; A is selected from the group consisting of CO₂Me and H; Z is selected from the group consisting of H and NO₂; and E is selected from the group consisting of CN, CO₂Me, OMe, and NO₂; G is independently at each occurrence selected from the group consisting of H, halogen, carbonyl, ester, ketone, amide, alkyl, cycloalkyl, aryl, arylalkyl, alkenyl, heteroalkyl, cyano, alkoxy, amine, haloalkyl, heteroatoms, and heterocycle; n is selected from the group consisting of 1, 2, 3, 4, 5, 6, 7, 8, 9, and 10; k is independently at each occurrence from 0 to 4; R is selected from the group consisting of methyl, ethyl, isopropyl, mesityl, and alkyl sulfonate; and R′ is selected from the group consisting of H, alkyl, cycloalkyl, halogen, alkenyl, aryl, arylalkyl, haloalkyl, heteroalkyl, acetyl, heterocycle, amine, amide, ether, and carbonyl, and any combination thereof; wherein said compound, optionally in addition to more than one molecule of said compound, is conjugated to a compound selected from the group consisting of a sugar, a heterocycle, an antibody, a minor groove binder, an intercalator, an amino acid, and a peptide. In certain embodiments, the conjugation is direct. In certain embodiments, the conjugation is via a linker. In certain embodiments, the linker is selected from the group consisting of alkyl, heteroatom, heteroalkyl, haloalkyl, alkenyl, alkoxy, ester, ether, amide, hydrazine, hydrazone, acetal, ketal, disulfide, phosphate, Mal-PEG-NHS ester, DBCO-NHS ester, and SMCC.

In certain embodiments, the compound is conjugated to a sugar. In certain embodiments, the compound has the structure selected from the group consisting of Formula (XV) and Formula (XVI):

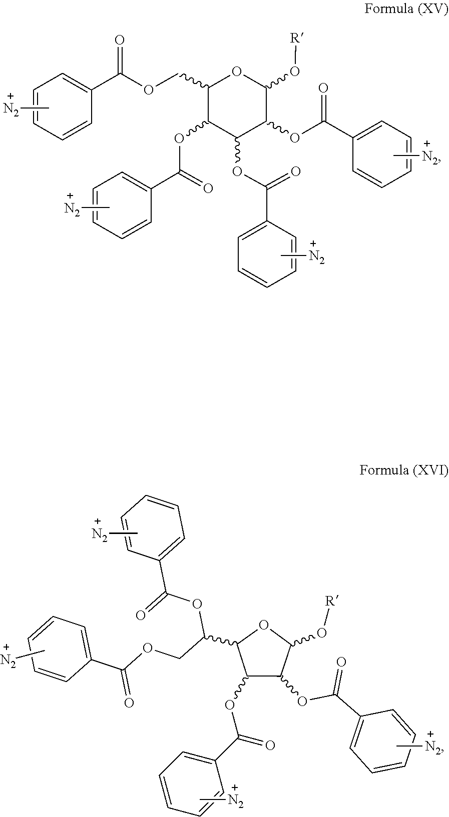

or a pharmaceutically acceptable salt thereof, wherein: R′ is selected from the group consisting of H, alkyl, cycloalkyl, halogen, alkenyl, aryl, arylalkyl, haloalkyl, heteroalkyl, acetyl, heterocycle, amine, amide, ether, carbonyl, maleimide (Mal)-polyethylene glycol (PEG)-N-Hydroxysuccinimide (NHS) ester, dibenzocyclooctyne (DBCO)—NHS ester, and succinimidyl 4-(N-maleimidomethyl)cyclohexane-1-carboxylate (SMCC), and any combination thereof. In certain embodiments, the compound is:

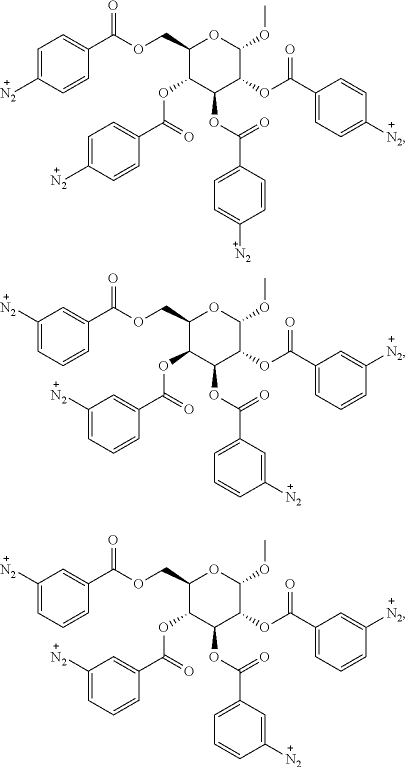

or a pharmaceutically acceptable salt thereof. In certain embodiments, the sugar is glucose, galactose, mannose, or fructose. In certain embodiments, the sugar is a pyranose. In certain embodiments, the sugar is a furanose. In certain embodiments, the sugar is a monosaccharide. In certain embodiments, the sugar is a disaccharide. In certain embodiments, the sugar is a polysaccharide.

In certain embodiments, the compound is conjugated to a heterocycle. In certain embodiments, the heterocycle is pyrrole, pyridine, indole, furan, imidazole, pyrazole, thiazole, or thiophene.

In certain embodiments, the compound is conjugated to an antibody. In certain embodiments, the antibody is an anti-CD33 antibody, an anti-CD22 antibody, or trastuzumab.

In certain embodiments, the compound is conjugated to a minor groove binder. In certain embodiments, the minor groove binder is netropsin or distamycin A.

In certain embodiments, the compound is conjugated to an intercalator. In certain embodiments, the intercalator is ethidium bromide, an anthracycline, or an acridine derivative.

In certain embodiments, the compound is conjugated to an amino acid.

In certain embodiments, the compound is conjugated to a peptide.

In certain embodiments, the compound is a salt comprising a counterion selected from the group consisting of tetrafluoroborate, hexafluorophosphate, tosylate, chloride, bromide, and hydrogen sulfate.

In certain embodiments, a pharmaceutical composition comprises the compound as described herein, or a pharmaceutically acceptable salt thereof.

In certain embodiments, a pharmaceutical dosage form comprises the compound as described herein, or the pharmaceutical composition as described herein, or a pharmaceutically acceptable salt thereof.

In certain embodiments, a method of cleaving DNA in a cell comprises contacting the cell with an effective amount of the compound as described herein, the pharmaceutical composition as described herein, or the pharmaceutical dosage as described herein.

In another aspect, provided herein is a method of cleaving DNA in a cell comprising contacting the cell with an effective amount of a compound according to Formula (XXIV):

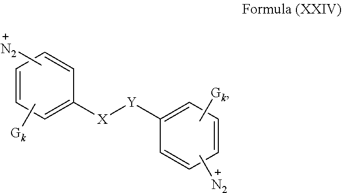

or a pharmaceutically acceptable sale thereof, wherein: X and Y are independently selected from the group consisting of bond, alkyl, S, Se, P, C═O, NH, NR′, S, O, amide, and ester; k is independently at each occurrence from 0 to 4; G is independently at each occurrence selected from the group consisting of H, halogen, carbonyl, ester, ketone, amide, alkyl, cycloalkyl, aryl, arylalkyl, alkenyl, heteroalkyl, cyano, alkoxy, amine, haloalkyl, heteroatoms, and heterocycle; R′ is selected from the group consisting of H, alkyl, cycloalkyl, halogen, alkenyl, aryl, arylalkyl, haloalkyl, heteroalkyl, acetyl, heterocycle, amine, amide, ether, and carbonyl, and any combination thereof.

In a further aspect, provided herein is a method of cleaving DNA in a cell comprising contacting the cell with an effective amount of a compound according to Formula (VI):

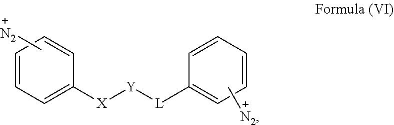

or a pharmaceutically acceptable salt thereof, wherein: X is selected from the group consisting of S, Se, P, bond, NH, NMe, and O; Y is selected from the group consisting of O and C═O; and L is selected from the group consisting of S, Se, P, bond, and NH. In certain embodiments, the compound is:

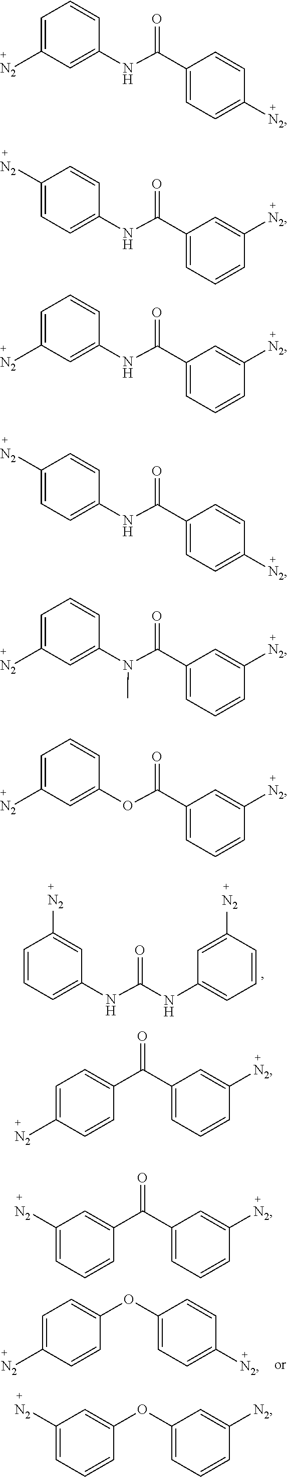

or a pharmaceutically acceptable salt thereof.

In another aspect, provided herein is a method of cleaving DNA in a cell comprising contacting the cell with an effective amount of a compound according to Formula (VII):

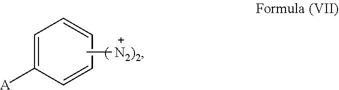

or a pharmaceutically acceptable salt thereof, wherein: A is selected from the group consisting of CO₂Me and H. In certain embodiments, the compound is:

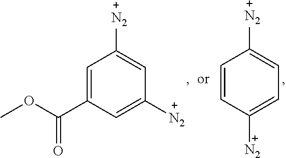

or a pharmaceutically acceptable salt thereof.

In a further aspect, provided herein is a method of cleaving DNA in a cell comprising contacting the cell with an effective amount of a compound selected from the group consisting of Formula (VIII), Formula (IX), Formula (X), Formula (XI), Formula (XII), and Formula (XXXII):

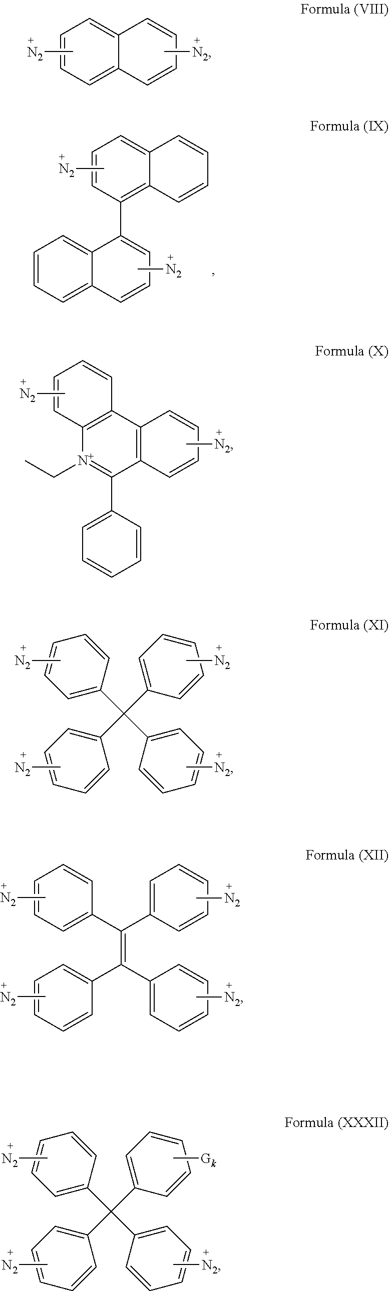

or a pharmaceutically acceptable salt thereof, wherein: k is independently at each occurrence from 0 to 4; G is independently at each occurrence selected from the group consisting of H, halogen, carbonyl, ester, ketone, amide, alkyl, cycloalkyl, aryl, arylalkyl, alkenyl, heteroalkyl, cyano, alkoxy, amine, haloalkyl, heteroatoms, and heterocycle. In certain embodiments, the compound is:

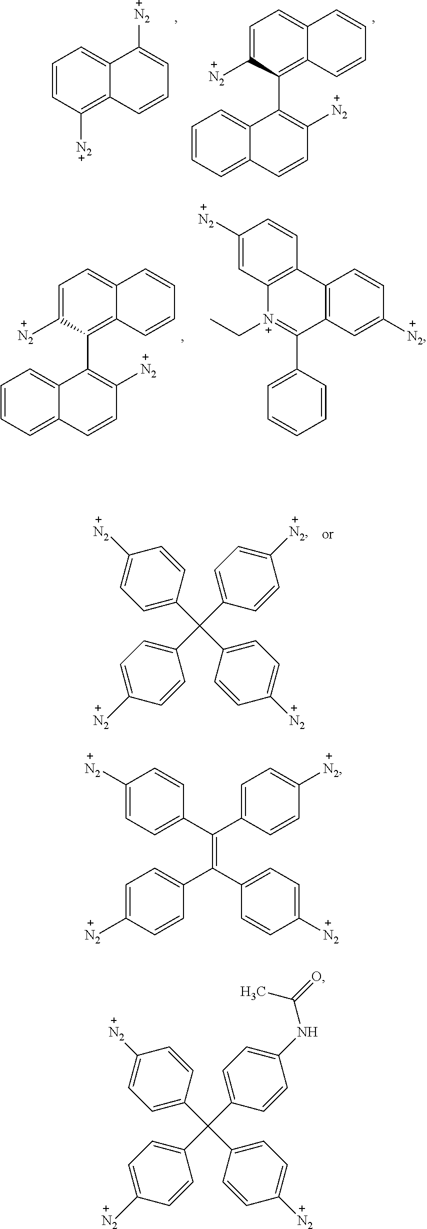

or a pharmaceutically acceptable salt thereof.

In another aspect, provided herein is a method of cleaving DNA in a cell comprising contacting the cell with an effective amount of a compound according to Formula (XIII):

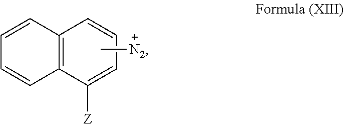

or a pharmaceutically acceptable salt thereof, wherein: Z is selected from the group consisting of H and NO₂. In certain embodiments, the compound is:

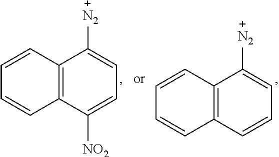

or a pharmaceutically acceptable salt thereof.

In a further aspect, provided herein is a method of cleaving DNA in a cell comprising contacting the cell with an effective amount of a compound according to Formula (XIV):

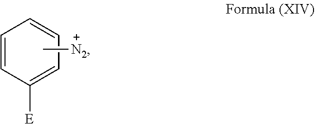

or a pharmaceutically acceptable salt thereof, wherein: E is selected from the group consisting of CN, CO₂Me, OMe, and NO₂. In certain embodiments, the compound is:

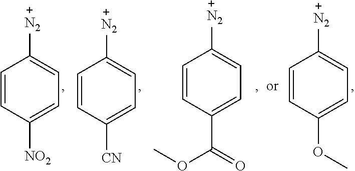

or a pharmaceutically acceptable salt thereof.

In certain embodiments, the compound is a salt comprising a counterion that is tetrafluoroborate.

In certain embodiments, the method further comprises a reduction step after contacting the cell with the compound. In certain embodiments, the reduction step comprises reduction of the compound to its corresponding radical upon nitrogen extrusion.

In certain embodiments, the reduction step comprises light irradiation. In certain embodiments, the light irradiation is performed with light of a wavelength comprising one or more wavelengths within the full spectrum of light wavelengths. In certain embodiments, the light irradiation is performed with light selected from the group consisting of ambient light, blue light at about 467 nm, green light at about 525 nm, red light at about 660 nm, and near-IR light at about 1040 nm to about 2500 nm. In certain embodiments, the reduction step is performed with spatial and temporal selectivity.

In certain embodiments, the reduction step comprises treatment of the compound with a reducing agent. In certain embodiments, the reducing agent is selected from the group consisting of NADPH, NADH, FADH₂, glutathione, cysteine, and amine reductants. In certain embodiments, the reducing agent is cysteine. In certain embodiments, the reducing agent is endogenous. In certain embodiments, the reducing agent is exogenous.

In certain embodiments, the reduction step comprises treatment of the compound with a metal reductant. In certain embodiments, the metal reductant is selected from the group consisting of copper (I) chloride, iron (II) sulfate, zinc metal, and iron metal.

In certain embodiments, the DNA cleavage comprises one or more double strand breaks in the DNA. In certain embodiments, the DNA cleavage comprises one or more single strand breaks in the DNA. In certain embodiments, the DNA comprises supercoiled double stranded DNA. In certain embodiments, the DNA comprises linear double stranded DNA.

In certain embodiments, the compound cleaves DNA with an EC₅₀ of less than 600 nM. In certain embodiments, the compound cleaves DNA with an EC₅₀ of less than 150 nM. In certain embodiments, the compound cleaves DNA with an EC₅₀ of less than 75 nM. In certain embodiments, the compound cleaves DNA with an EC₅₀ of less than 25 nM. In certain embodiments, the compound cleaves DNA with an EC₅₀ of less than 15 nM. In certain embodiments, the compound cleaves DNA with an EC₅₀ of less than 10 nM. In certain embodiments, the compound cleaves DNA with an EC₅₀ of less than 5 nM. In certain embodiments, the compound cleaves DNA with an EC₅₀ of less than 3 nM.

In certain embodiments, the cell is a bacterial cell. In certain embodiments, the cell is a mammalian cell. In certain embodiments, the mammalian cell is a tumor cell.

In a further aspect, provided herein is a method of treating a bacterial infection in a subject in need thereof, comprising administering to the subject an effective amount of the compound as described herein, the pharmaceutical composition as described herein, or the pharmaceutical dosage as described herein. In certain embodiments, the method of treating a bacterial infection in a subject in need thereof, comprises administering to the subject an effective amount of a compound selected from the group consisting of Formula (I), Formula (II), Formula (III), Formula (IV), Formula (V), Formula (VI), Formula (VII), Formula (VIII), Formula (IX), Formula (X), Formula (XI), Formula (XII), Formula (XIII), Formula (XIV), Formula (XV), Formula (XVI), Formula (XVII), Formula (XVIII), Formula (XIX), Formula (XX), Formula (XXI), Formula (XXII), Formula (XXIII), Formula (XXIV), Formula (XXV), Formula (XXVI), Formula (XXVII), Formula (XXVIII), Formula (XXIX), Formula (XXX), Formula (XXXI), and Formula (XXXII).

In another aspect, provided herein is a method of treating a viral infection in a subject in need thereof, comprising administering to the subject an effective amount of the compound as described herein, the pharmaceutical composition as described herein, or the pharmaceutical dosage as described herein. In certain embodiments, the method of treating a viral infection in a subject in need thereof, comprises administering to the subject an effective amount of a compound selected from the group consisting of Formula (I), Formula (II), Formula (III), Formula (IV), Formula (V), Formula (VI), Formula (VII), Formula (VIII), Formula (IX), Formula (X), Formula (XI), Formula (XII), Formula (XIII), Formula (XIV), Formula (XV), Formula (XVI), Formula (XVII), Formula (XVIII), Formula (XIX), Formula (XX), Formula (XXI), Formula (XXII), Formula (XXIII), Formula (XXIV), Formula (XXV), Formula (XXVI), Formula (XXVII), Formula (XXVIII), Formula (XXIX), Formula (XXX), Formula (XXXI), and Formula (XXXII).

In a further aspect, provided herein is a method of treating a cancer in a subject in need thereof, comprising administering to the subject an effective amount of the compound as described herein, the pharmaceutical composition as described herein, or the pharmaceutical dosage as described herein. In certain embodiments, the method of treating a cancer in a subject in need thereof, comprises administering to the subject an effective amount of a compound selected from the group consisting of Formula (I), Formula (II), Formula (III), Formula (IV), Formula (V), Formula (VI), Formula (VII), Formula (VIII), Formula (IX), Formula (X), Formula (XI), Formula (XII), Formula (XIII), Formula (XIV), Formula (XV), Formula (XVI), Formula (XVII), Formula (XVIII), Formula (XIX), Formula (XX), Formula (XXI), Formula (XXII), Formula (XXIII), Formula (XXIV), Formula (XXV), Formula (XXVI), Formula (XXVII), Formula (XXVIII), Formula (XXIX), Formula (XXX), Formula (XXXI), and Formula (XXXII). In certain embodiments, the cancer is a solid tumor. In certain embodiments, the cancer is selected from the group consisting of kidney cancer, adrenal gland cancer, anal cancer, appendiceal cancer, ovarian cancer, uterine cancer, gastric cancer, breast cancer, lung cancer, bladder cancer, cervical cancer, stomach cancer, sarcoma cancer, liver cancer, esophageal cancer, laryngeal cancer, multiple myeloma, colorectal cancer, rectal cancer, skin cancer, pancreatic cancer, brain or spinal cord cancer, leukemia or lymphoma. In certain embodiments, the cancer is breast cancer or human lung carcinoma.

In certain embodiments, the subject is a human.

In certain embodiments, the compound is administered to the subject orally, topically, intranasally, intravenously, intramuscularly, or subcutaneously.

### BRIEF DESCRIPTION OF THE DRAWINGS

FIGS. 1A-1B show the DNA-cleaving ability of compound 1. FIG. 1A shows the agarose gel images of DNA incubated with compound 1 under green light irradiation. FIG. 1B shows the dose response plot of cleaved DNA percentage vs. concentration of compound 1.

FIGS. 2A-2B show the DNA-cleaving ability of compound 2. FIG. 2A shows the agarose gel images of DNA incubated with compound 2 under green light irradiation. FIG. 2B shows the dose response plot of cleaved DNA percentage vs. concentration of compound 2.

FIGS. 3A-3B show the DNA-cleaving ability of compound 3. FIG. 3A shows the agarose gel images of DNA incubated with compound 3 under green light irradiation. FIG. 3B shows the dose response plot of cleaved DNA percentage vs. concentration of compound 3.

FIGS. 4A-4B show the DNA-cleaving ability of compound 4. FIG. 4A shows the agarose gel images of DNA incubated with compound 4 under green light irradiation. FIG. 4B shows the dose response plot of cleaved DNA percentage vs. concentration of compound 4.

FIGS. 5A-5B show the DNA-cleaving ability of compound 5. FIG. 5A shows the agarose gel images of DNA incubated with compound 5 under green light irradiation. FIG. 5B shows the dose response plot of cleaved DNA percentage vs. concentration of compound 5.

FIGS. 6A-6B show the DNA-cleaving ability of compound 6. FIG. 6A shows the agarose gel images of DNA incubated with compound 6 under green light irradiation. FIG. 6B shows the dose response plot of cleaved DNA percentage vs. concentration of compound 6.

FIGS. 7A-7B show the DNA-cleaving ability of compound 7. FIG. 7A shows the agarose gel images of DNA incubated with compound 7 under green light irradiation. FIG. 7B shows the dose response plot of cleaved DNA percentage vs. concentration of compound 7.

FIGS. 8A-8B show the DNA-cleaving ability of compound 8. FIG. 8A shows the agarose gel images of DNA incubated with compound 8 under green light irradiation. FIG. 8B shows the dose response plot of cleaved DNA percentage vs. concentration of compound 8.

FIGS. 9A-9B show the DNA-cleaving ability of compound 9. FIG. 9A shows the agarose gel images of DNA incubated with compound 9 under green light irradiation. FIG. 9B shows the dose response plot of cleaved DNA percentage vs. concentration of compound 9.

FIGS. 10A-10B show the DNA-cleaving ability of compound 10. FIG. 10A shows the agarose gel images of DNA incubated with compound 10 under green light irradiation.

FIG. 10B shows the dose response plot of cleaved DNA percentage vs. concentration of compound 10.

FIGS. 11A-11B show the DNA-cleaving ability of compound 11. FIG. 11A shows the agarose gel images of DNA incubated with compound 11 under green light irradiation.

FIG. 11B shows the dose response plot of cleaved DNA percentage vs. concentration of compound 11.

FIGS. 12A-12B show the DNA-cleaving ability of compound 12. FIG. 12A shows the agarose gel images of DNA incubated with compound 12 under green light irradiation.

FIG. 12B shows the dose response plot of cleaved DNA percentage vs. concentration of compound 12.

FIGS. 13A-13B show the DNA-cleaving ability of compound 13. FIG. 13A shows the agarose gel images of DNA incubated with compound 13 under green light irradiation.

FIG. 13B shows the dose response plot of cleaved DNA percentage vs. concentration of compound 13.

FIGS. 14A-14B show the DNA-cleaving ability of compound 14. FIG. 14A shows the agarose gel images of DNA incubated with compound 14 under green light irradiation.

FIG. 14B shows the dose response plot of cleaved DNA percentage vs. concentration of compound 14.

FIGS. 15A-15B show the DNA-cleaving ability of compound 15. FIG. 15A shows the agarose gel images of DNA incubated with compound 15 under green light irradiation.

FIG. 15B shows the dose response plot of cleaved DNA percentage vs. concentration of compound 15.

FIGS. 16A-16B show the DNA-cleaving ability of compound 16. FIG. 16A shows the agarose gel images of DNA incubated with compound 16 under green light irradiation.

FIG. 16B shows the dose response plot of cleaved DNA percentage vs. concentration of compound 16.

FIGS. 17A-17B show the DNA-cleaving ability of compound 17. FIG. 17A shows the agarose gel images of DNA incubated with compound 17 under green light irradiation.

FIG. 17B shows the dose response plot of cleaved DNA percentage vs. concentration of compound 17.

FIGS. 18A-18B show the DNA-cleaving ability of compound 18. FIG. 18A shows the agarose gel images of DNA incubated with compound 18 under green light irradiation.

FIG. 18B shows the dose response plot of cleaved DNA percentage vs. concentration of compound 18.

FIGS. 19A-19B show the DNA-cleaving ability of compound 19. FIG. 19A shows the agarose gel images of DNA incubated with compound 19 under green light irradiation.

FIG. 19B shows the dose response plot of cleaved DNA percentage vs. concentration of compound 19.

FIGS. 20A-20B show the DNA-cleaving ability of compound 20. FIG. 20A shows the agarose gel images of DNA incubated with compound 20 under green light irradiation.

FIG. 20B shows the dose response plot of cleaved DNA percentage vs. concentration of compound 20.

FIGS. 21A-21B show the DNA-cleaving ability of compound 21. FIG. 21A shows the agarose gel images of DNA incubated with compound 21 under green light irradiation.

FIG. 21B shows the dose response plot of cleaved DNA percentage vs. concentration of compound 21.

FIGS. 22A-22B show the DNA-cleaving ability of compound 22. FIG. 22A shows the agarose gel images of DNA incubated with compound 22 under green light irradiation.

FIG. 22B shows the dose response plot of cleaved DNA percentage vs. concentration of compound 22.

FIGS. 23A-23B show the DNA-cleaving ability of compound 23. FIG. 23A shows the agarose gel images of DNA incubated with compound 23 under green light irradiation.

FIG. 23B shows the dose response plot of cleaved DNA percentage vs. concentration of compound 23.

FIGS. 24A-24B show the DNA-cleaving ability of compound 24. FIG. 24A shows the agarose gel images of DNA incubated with compound 24 under green light irradiation. FIG. 24B shows the dose response plot of cleaved DNA percentage vs. concentration of compound 24.

FIGS. 25A-25B show the DNA-cleaving ability of compound 25. FIG. 25A shows the agarose gel images of DNA incubated with compound 25 under green light irradiation.

FIG. 25B shows the dose response plot of cleaved DNA percentage vs. concentration of compound 25.

FIGS. 26A-26B show the DNA-cleaving ability of compound 26. FIG. 26A shows the agarose gel images of DNA incubated with compound 26 under green light irradiation.

FIG. 26B shows the dose response plot of cleaved DNA percentage vs. concentration of compound 26.

FIGS. 27A-27B show the DNA-cleaving ability of compound 27. FIG. 27A shows the agarose gel images of DNA incubated with compound 27 under green light irradiation.

FIG. 27B shows the dose response plot of cleaved DNA percentage vs. concentration of compound 27.

FIGS. 28A-28B show the DNA-cleaving ability of compound 28. FIG. 28A shows the agarose gel images of DNA incubated with compound 28 under green light irradiation.

FIG. 28B shows the dose response plot of cleaved DNA percentage vs. concentration of compound 28.

FIGS. 29A-29B show the DNA-cleaving ability of compound 29. FIG. 29A shows the agarose gel images of DNA incubated with compound 29 under green light irradiation.

FIG. 29B shows the dose response plot of cleaved DNA percentage vs. concentration of compound 29.

FIGS. 30A-30B show the DNA-cleaving ability of compound 30. FIG. 30A shows the agarose gel images of DNA incubated with compound 30 under green light irradiation.

FIG. 30B shows the dose response plot of cleaved DNA percentage vs. concentration of compound 30.

FIGS. 31A-31B show the DNA-cleaving ability of compound 13 using low-energy red light. FIG. 31A shows the agarose gel images of DNA incubated with compound 13 under low-energy red light irradiation. FIG. 31B shows the dose response plot of cleaved DNA percentage vs. concentration of compound 13 using low-energy red light.

FIGS. 32A-32B show the DNA-cleaving ability of compound 28 using ambient light. FIG. 32A shows the agarose gel image of DNA incubated with compound 28 under ambient light. FIG. 32B shows the dose response plot of cleaved DNA percentage vs. concentration of compound 28 using ambient light.

FIGS. 33A-33B show the DNA-cleaving ability of compound 28 in the presence of cysteine in the dark. FIG. 33A shows the agarose gel image of DNA incubated with compound 28 under the presence of cysteine in the dark. FIG. 33B shows the dose response plot of cleaved DNA percentage vs. concentration of compound 28.

FIGS. 34A-34B show the DNA-cleaving ability of compound 31. FIG. 34A shows the agarose gel images of DNA incubated with compound 31 under green light irradiation.

FIG. 34B shows the dose response plot of cleaved DNA percentage vs. concentration of compound 31.

FIGS. 35A-35B show the DNA-cleaving ability of compound 32. FIG. 35A shows the agarose gel images of DNA incubated with compound 32 under green light irradiation.

FIG. 35B shows the dose response plot of cleaved DNA percentage vs. concentration of compound 32.

FIGS. 36A-36B show the DNA-cleaving ability of compound 33. FIG. 36A shows the agarose gel images of DNA incubated with compound 33 under green light irradiation.

FIG. 36B shows the dose response plot of cleaved DNA percentage vs. concentration of compound 33.

FIGS. 37A-37B show the DNA-cleaving ability of compound 28 using blue light at 467 nm. FIG. 37A shows the agarose gel images of DNA incubated with compound 28 under blue light irradiation. FIG. 37B shows the dose response plot of cleaved DNA percentage vs. concentration of compound 28 using blue light.

FIGS. 38A-38B show the DNA-cleaving ability of compound 28 using low-energy red light at 660 nm. FIG. 38A shows the agarose gel images of DNA incubated with compound 28 under low-energy red light irradiation. FIG. 38B shows the dose response plot of cleaved DNA percentage vs. concentration of compound 28 using low-energy red light.

FIGS. 39A-39B show the DNA-cleaving ability of compound 28 on linearized double stranded DNA. FIG. 39A shows the agarose gel images of linearized double stranded DNA incubated with compound 28 under green light irradiation. For the gel on the left: Lanes 1-8 show purified EcoRV-HF-digested pBR322 (0.5 μg) and corresponding solution of diazonium compound 28; Lane 9 shows purified EcoRV-HF-digested pBR322 (0.5 μg) with no diazonium nor light irradiation; Lane 10 shows supercoiled pBR322 plasmid (0.5 μg) with no diazonium nor light irradiation. The gel on the right shows the assay with use of supercoiled pBR322 as an internal standard (0.5 μg): Lanes 1-8 show purified EcoRV-HF-digested pBR322 (0.5 μg) and corresponding solution of diazonium 28 and added supercoiled pBR322 (0.5 μg); Lane 9 shows a mix of purified EcoRV-HF-digested pBR322 (0.5 μg) and supercoiled pBR322 plasmid (0.5 μg) with no diazonium nor light irradiation; Lane 10 shows purified EcoRV-HF-digested pBR322 (0.5 μg) with no diazonium nor light irradiation; Lane 11 shows supercoiled pBR322 plasmid (0.5 μg) with no diazonium nor light irradiation. FIG. 39B shows the dose response plot of cleaved DNA percentage vs. concentration of compound 28 on linearized pBR322 with supercoiled pBR322 as an internal standard.

FIGS. 40A-40B show the antiproliferation activity of compound 28 on live HeLa cells. FIG. 40A shows confocal microscopy images of the HeLa cells after treatment with compound 28. Image 1 shows a control plate of HeLa cells incubated with DMEM in the absence of compound 28. The plate is nearly completely confluent after 3 days. Image 2 shows a control plate of HeLa cells incubated with 1×PBS pH 6.6 in the absence of compound 28. The plate is nearly completely confluent after 3 days, similar to the plate incubated with DMEM. Image 3 shows a plate of HeLa cells incubated with 1.25 μM of compound 28. Plate is over 90% confluent after 3 days, showing minimal cytotoxicity at this concentration, consistent with the IC₅₀ plot. Image 4 shows a plate of HeLa cells incubated with 10 μM of compound 28. Plate is less than 10% confluent after 3 days, consisting of mainly cellular debris, demonstrating effective cytotoxicity of compound 28 at this concentration, consistent with the IC₅₀ plot. FIG. 40B shows the cell viability dose response plot of cell viability percentage vs. concentration of compound 28.

FIGS. 41A-41B show the DNA-cleaving ability of compound 28 with deoxygenated water. FIG. 41A shows the agarose gel images of DNA incubated with compound 28 with degassed deionized water (1) and standard deionized water (2). FIG. 41B shows the dose response plot of cleaved DNA percentage vs. concentration of compound 28 in degassed deionized water (1) and standard deionized water (2).

FIG. 42 shows the results of UV-Vis (ultraviolet-visible spectroscopy) studies of compound 28. Absorbances were measured for supercoiled pBR322 in water, linearized (EcoRV-HF-digested) pBR322 in water, compound 28 in water, compound 28 and pBR322 immediately after preparation, compound 28 and pBR322 after left in the dark for 20 minutes, compound 28 and linearized pBR322, and compound 28 and supercoiled pBR322 after irradiation with 525 nm light for 20 minutes.

FIG. 43 shows an agarose gel image of DNA cleavage assay controls with restriction enzymes and amine precursor. Lane 1: pBR322 incubated with nicking enzyme Nb.BtsI; Lane 2: pBR322 irradiated in the presence of 500 nM of diazonium compound 10; Lane 3: pBR322 incubated with restriction enzyme EcoRV-HF; Lane 4: pBR322 irradiated in the presence of 500 nM of 1,5-diaminonaphthalene; Lane 5: pBR322 irradiated with water alone; Lane 6: pBR322 with no irradiation.

FIGS. 44A-44B show the DNA-cleaving ability of compound 28 in the dark. FIG. 44A shows the agarose gel images of DNA incubated with compound 28 in the dark. FIG. 44B shows the dose response plot of cleaved DNA percentage vs. concentration of compound 28 in the dark.

FIG. 45 shows the NMR spectra of compound 28 in D₂O at different time points. Spectra showed: (T₀) immediately after sample preparation of compound 28 with a 28/IS ratio of 0.68; (1 h) at 1 hour with a 28/IS ratio of 0.68; (2 h) at 2 hours with a 28/IS ratio of 0.67; (6 h) at 6 hours with a 28/IS ratio of 0.64; (24 h) at 24 hours with a 28/IS ratio of 0.54.

FIG. 46 shows the NMR spectra of compound 10 in D₂O at different time points. Spectra showed: (T₀) immediately after sample preparation of compound 10 with a 10/IS ratio of 0.62; (1 h) at 1 hour with a 10/IS ratio of 0.59; (2 h) at 2 hours with a 10/IS ratio of 0.56; (6 h) at 6 hours with a 10/IS ratio of 0.49; (24 h) at 24 hours with a 10/IS ratio of 0.41.

FIGS. 47A-47B show the DNA-cleaving ability of compound 34. FIG. 47A shows the agarose gel images of DNA incubated with compound 34 under green light irradiation.

FIG. 47B shows the dose response plot of cleaved DNA percentage vs. concentration of compound 34.

### DETAILED DESCRIPTION

Detailed embodiments of the present invention are disclosed herein; however, it is to be understood that the disclosed embodiments are merely illustrative of the invention that may be embodied in various forms. In addition, each of the examples given in connection with the various embodiments of the invention is intended to be illustrative, and not restrictive. Therefore, specific structural and functional details disclosed herein are not to be interpreted as limiting, but merely as a representative basis for teaching one skilled in the art to variously employ the present invention.

Inspired by the high potency of (−)-lomaiviticin, it was hypothesized that the active species for DNA cleavage, benzenoid radicals, could be generated from diazonium salts through photoredox activation upon nitrogen extrusion (Behr, 1989; Hari, et al., 2012; Romero, et al., 2016). Aryl diazonium salts present as a more accessible and tunable precursor to aryl radicals. The aryl diazonium compounds as described herein are readily available and inexpensive as they can be synthesized from commercial materials, aryl amine precursors, in 1-3 simple steps vs. the 36-step total synthesis of calicheamicin γ₁. The straightforward synthesis and the ease of modification offer a broad range of functionalities and structural motifs to further establish the structure-activity-relationship (SAR). Aryl diazonium salts showed a wide range of potencies in DNA cleavage, with EC₅₀ values ranging from low nanomolar to micromolar. Furthermore, integrating machine learning models can facilitate the development of even more potent derivatives.

Aryl diazonium salts are reduced by mild reductants, including low-energy, long-wavelength light, producing aryl radicals upon nitrogen extrusion (E=−0.16 V/SCE) (Andrieux, et al., 2003). Photoredox conditions can facilitate the formation of aryl radicals by the reduction of aryl diazonium salts (Hari, et al., 2012; Romero, et al., 2016). While diazonium salts have been studied in the context of nucleophilic aromatic substitution reactions and recently in photoredox activation, the studies as described herein have unveiled new reactivity. Without wishing to be bound by theory, it is postulated that an electron donor-acceptor (EDA) interaction may form between a diazonium and an electron-rich moiety present in DNA and peptides.

This EDA interaction facilitated a light-induced electron transfer even without the presence of a photocatalyst, and intriguingly, was initiated solely by light, including ambient light, blue light (467 nm), green light (525 nm), low-energy red light (660 nm), and near-IR light (>1040 nm). The ability for aryl diazoniums to be activated with low-energy light irradiation can allow for enhanced specificity through precise spatial and temporal control over the generation of these aryl radicals. The conditions identified in the reactions described herein are mild, physiologically compatible conditions. The reactions proceed at a fast reaction rate and are compatible with an aqueous solution, bypassing the need for any organic co-solvent.

Forming benzenoid diradicals does not appear to be necessary for potent DNA cleavage by the diazonium compounds; instead, DNA cleavage can be achieved with radicals distributed among different arenes when connected with proper linkages. The potency is influenced by electronic effects, stereochemistry, orbital orientations, the distance between multi-radicals, and the number of diazonium motifs within the molecule.

It was found that multi-diazonium compounds, despite their structural simplicity, can reach a greater potency of DNA cleavage (EC₅₀=2.76 nM) compared to calicheamicin γ₁ (EC₅₀=7-10 nM). There was a significant dependence between the potency of the diazonium compound and the overall structure. Such small molecule DNA cleavage agents can serve as active cytotoxic components in novel antitumor drugs, providing a more cost-effective, efficient, potent, and modular alternative to calicheamicin γ₁ as an approach to cancer treatment. The utilization of red-light-activatable compounds holds great potential for integration into phototherapy, in which the drug is activated through irradiation specifically at the tumor site. This approach enables targeted treatment and minimizes the impact on healthy surrounding tissues. Further, the diazonium compounds as disclosed herein are versatile to enable conjugation with molecular recognition components.

### Definitions

To facilitate an understanding of the principles and features of the various embodiments of the invention, various illustrative embodiments are explained below. Although exemplary embodiments of the invention are explained in detail, it is to be understood that other embodiments are contemplated. Accordingly, it is not intended that the invention is limited in its scope to the details of construction and arrangement of components set forth in the following description or examples. The invention is capable of other embodiments and of being practiced or carried out in various ways. Also, in describing the exemplary embodiments, specific terminology will be resorted to for the sake of clarity.

As used in this specification and the appended claims, the singular forms “a”, “an”, and “the” include plural references unless the context clearly dictates otherwise. Thus, for example, a reference to “a method” includes one or more methods, and/or steps of the type described herein and/or which will become apparent to those persons skilled in the art upon reading this disclosure.

The terms “treat”, “treating”, or “treatment” of a state, disorder or condition include: (1) preventing, delaying, or reducing the incidence and/or likelihood of the appearance of at least one clinical or sub-clinical symptom of the state, disorder or condition developing in a subject that may be afflicted with or predisposed to the state, disorder or condition but does not yet experience or display clinical or subclinical symptoms of the state, disorder or condition; or (2) inhibiting the state, disorder or condition, i.e., arresting, reducing or delaying the development of the disease or a relapse thereof (in case of maintenance treatment) or at least one clinical or sub-clinical symptom thereof; or (3) relieving the disease, i.e., causing regression of the state, disorder or condition or at least one of its clinical or sub-clinical symptoms. The benefit to a subject to be treated is either statistically significant or at least perceptible to the patient or to the physician.

As used herein the term “effective” applied to dose or amount refers to that quantity of a compound or pharmaceutical composition that is sufficient to result in a desired activity upon administration to a subject in need thereof. Note that when a combination of active ingredients is administered, the effective amount of the combination may or may not include amounts of each ingredient that would have been effective if administered individually. The exact amount required will vary from subject to subject, depending on the species, age, and general condition of the subject, the severity of the condition being treated, the particular drug or drugs employed, the mode of administration, and the like.

In the context of the field of medicine, the term “prevent” encompasses any activity which reduces the burden of mortality or morbidity from disease. Prevention can occur at primary, secondary and tertiary prevention levels. While primary prevention avoids the development of a disease, secondary and tertiary levels of prevention encompass activities aimed at preventing the progression of a disease and the emergence of symptoms as well as reducing the negative impact of an already established disease by restoring function and reducing disease-related complications.

The phrase “pharmaceutically acceptable”, as used in connection with compositions of the invention, refers to molecular entities and other ingredients of such compositions that are physiologically tolerable and do not typically produce untoward reactions when administered to a mammal (e.g., a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans.

As used herein, the term “pharmaceutically acceptable salt” means those salts of compounds of the invention that are safe for application in a subject. In certain embodiments, the pharmaceutically acceptable salts include salts of acidic or basic groups present in compounds of the present disclosure. Pharmaceutically acceptable acid salts include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,11-methylene-bis-(2-hydroxy-3-naphthoate)) salts. Certain compounds of the present disclosure can form pharmaceutically acceptable salts with various amino acids. Suitable base salts include, but are not limited to, aluminum, calcium, lithium, magnesium, potassium, sodium, zinc, and diethanolamine salts. Lists of suitable salts are found in Remington's Pharmaceutical Sciences, 17th ed., Mack Publishing Company, Easton, Pa., 1985, p. 1418 and Berge, S M et al, Journal of Pharmaceutical Science, 1977, 66, 1, 1-19.

The terms “patient”, “individual”, “subject”, and “animal” are used interchangeably herein and refer to mammals, including, without limitation, human and veterinary animals (e.g., cats, dogs, cows, horses, goats, sheep, pigs, etc.) and experimental animal models. In a preferred embodiment, the subject is a human.

The term “about” or “approximately” means within a statistically meaningful range of a value. Such a range can be within an order of magnitude, preferably within 50%, more preferably within 20%, still more preferably within 10%, and even more preferably within 5% of a given value or range. The allowable variation encompassed by the term “about” or “approximately” depends on the particular system under study, and can be readily appreciated by one of ordinary skill in the art.

By “comprising” or “containing” or “including” is meant that at least the named compound, element, particle, or method step is present in the composition or article or method, but does not exclude the presence of other compounds, materials, particles, or method steps, even if the other such compounds, material, particles, or method steps have the same function as what is named.

Compounds of the present invention include those described generally herein, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausalito: 1999, and “March's Advanced Organic Chemistry”, 5th Ed., Ed.: Smith, M. B. and March, J., John Wiley & Sons, New York: 2001, the entire contents of which are hereby incorporated by reference.

The term “aryl” used alone or as part of a larger moiety as in “aralkyl,” “aralkoxy,” or “aryloxyalkyl,” refers to an aromatic monocyclic or polycyclic ring system containing from 6 to 19 (6, 7, 8, 9, 10, 11, 12, 13, 14, 15, 16, 17, 18, 19, 6-7, 6-8, 6-9, 6-10, 6-11, 6-12, 6-13, 6-14, 6-15, 6-16, 1-17, 6-18, 7-8, 7-9, 7-10, 7-11, 7-12, 7-13, 7-14, 7-15, 7-16, 7-18, 7-19, 8-9, 8-10, 8-11, 8-12, 8-13, 8-14, 8-15, 8-16, 8-17, 8-18, 8-19, 9-10, 9-11, 9-12, 9-13, 9-14, 9-15, 9-16, 9-17, 9-18, 9-19, 10-11, 10-12, 10-13, 10-14, 10-15, 10-16, 10-17, 10-18, 10-19, 11-12, 11-13, 11-14, 11-15, 11-16, 11-17, 11-18, 11-19, 12-13, 12-14, 12-15, 12-16, 12-17, 12-18, 12-19, 13-14, 13-15, 13-16, 13-17, 13-18, 13-19, 14-15, 14-16, 14-17, 14-18, 14-19, 15-16, 15-17, 15-18, 15-19, 16-17, 16-18, 16-19, 17-18, 17-19, 18-19) carbon atoms, where the ring system may be optionally substituted. Aryl groups of the present invention include, but are not limited to, groups such as phenyl, naphthyl, azulenyl, phenanthrenyl, anthracenyl, fluorenyl, pyrenyl, triphenylenyl, chrysenyl, and naphthacenyl.

As used herein, the term “alkyl” means an aliphatic hydrocarbon group which may be straight or branched having about 1 to about 8 (e.g., 1-2, 1-3, 1-4, 1-5, 1-6, 1-7, 1-8) carbon atoms in the chain. Branched means that one or more lower alkyl groups such as methyl, ethyl, or propyl are attached to a linear alkyl chain. Exemplary alkyl groups include methyl, ethyl, n-propyl, i-propyl, n-butyl, t-butyl, n-pentyl, and 3-pentyl.

The term “alkenyl” means an aliphatic hydrocarbon group containing a carbon-carbon double bond and which may be straight or branched having about 2 to about 8 (e.g., 2-3, 2-4, 2-5, 2-6, 2-7, 2-8) carbon atoms in the chain. Preferred alkenyl groups have 2 to about 4 carbon atoms in the chain. Exemplary alkenyl groups include ethenyl, propenyl, n-butenyl, and i-butenyl.

As used herein, the term “cycloalkyl” refers to a non-aromatic saturated or unsaturated mono- or polycyclic ring system which may contain 3 to 8 (3, 4, 5, 6, 7, 8, 3-4, 3-5, 3-6, 3-7, 4-5, 4-6, 4-7, 4-8, 5-6, 5-7, 5-8, 6-7, 6-8, 7-8) carbon atoms, and which may include at least one double bond. Exemplary cycloalkyl groups include, without limitation, cyclopropyl, cyclobutyl, cyclopentyl, cyclohexyl, cyclopropenyl, cyclobutenyl, cyclopentenyl, cyclohexenyl, anti-bicyclopropane, or syn-bicyclopropane.

As used herein, the terms “heterocycle,” “heterocyclyl,” “heterocyclic radical,” and “heterocyclic ring” are used interchangeably and refer to a stable 3- to 18-membered (3-, 4-, 5-, 6-, 7-, 8-, 9-, 10-, 11-, 12-, 13-, 14-, 15-, 16-, 17-, or 18-membered) ring system that consists of carbon atoms and from one to five (1, 2, 3, 4, 5, 1-2, 1-3, 1-4, 2-3, 2-4, 2-5, 3-4, 3-5, 4-5) heteroatoms selected from the group consisting of nitrogen, oxygen, and sulfur. The heterocyclyl may be a monocyclic or a polycyclic ring system, which may include fused, bridged, or spiro ring systems; and the nitrogen, carbon, or sulfur atoms in the heterocyclyl may be optionally oxidized; the nitrogen atom may be optionally quaternized; and the ring may be partially or fully saturated. Representative monocyclic heterocyclyls include piperidine, piperazine, pyrimidine, morpholine, thiomorpholine, pyrrolidine, tetrahydrofuran, pyran, tetrahydropyran, oxetane, and the like. Representative polycyclic heterocyclyls include indole, isoindole, indolizine, quinoline, isoquinoline, purine, carbazole, dibenzofuran, chromene, xanthene, and the like.

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be monocyclic, bicyclic, tricyclic, tetracyclic, and/or otherwise polycyclic. The term “heterocyclylalkyl” refers to an alkyl group substituted by a heterocyclyl, wherein the alkyl and heterocyclyl portions independently are optionally substituted.

As used herein, “heteroaryl” refers to an aromatic ring radical which consists of carbon atoms and from one to five heteroatoms selected from the group consisting of nitrogen, oxygen, and sulfur. Examples of heteroaryl groups include, without limitation, pyrrolyl, pyrazolyl, imidazolyl, triazolyl, furyl, thiophenyl, oxazolyl, isoxazolyl, thiazolyl, isothiazolyl, oxadiazolyl, thiadiazolyl, pyridyl, pyrazinyl, pyrimidinyl, pyridazinyl, triazinyl, thienopyrrolyl, furopyrrolyl, indolyl, azaindolyl, isoindolyl, indolinyl, indolizinyl, indazolyl, benzimidazolyl, imidazopyridinyl, benzotriazolyl, benzoxazolyl, benzoxadiazolyl, benzothiazolyl, pyrazolopyridinyl, triazolopyridinyl, thienopyridinyl, benzothiadiazolyl, benzofuyl, benzothiophenyl, quinolinyl, isoquinolinyl, tetrahydroquinolyl, tetrahydroisoquinolyl, cinnolinyl, quinazolinyl, quinolizilinyl, phthalazinyl, benzotriazinyl, chromenyl, naphthyridinyl, acrydinyl, phenanzinyl, phenothiazinyl, phenoxazinyl, pteridinyl, and purinyl. Additional heteroaryls are described in Comprehensive Heterocyclic Chemistry: The Structure, Reactions, Synthesis and Use of Heterocyclic Compounds (Katritzky et al. eds., 1984), which is hereby incorporated by reference in its entirety.

The term “arylalkyl” refers to a moiety of the formula —R^{a}R^{b} where R^{a} is an alkyl or cycloalkyl as defined above and R^{b} is an aryl or heteroaryl as defined above.

The term “ether” means a group having the formula —R—O—R—. Each R can be independently selected from the group consisting of C₁₋₈ alkylene, C₂₋₈ alkenylene, arylene, and heteroarylene. Exemplary ethers include, but are not limited to, —C₁₋₈ alkylene-O—C₁₋₈ alkylene- (e.g., —(CH₂)₂—O—(CH₂)₂—), —C₂₋₈ alkenylene-O—C₂₋₈ alkenylene-, -arylene-O-arylene-, -heteroarylene-O-heteroarylene-, and —C₁₋₈ alkylene-O-heteroarylene-.

The term “amide” means a group having the formula —C(O)N(R¹)(R¹) or —C(O)N(R¹)—. Amides include, e.g., —C(O)N(R¹)R—, —R—C(O)N(R¹)R—, —CHR¹—C(O)N(R¹)R—, and —C(R¹)(R¹)—C(O)N(R¹)R—. Each R can be independently selected from the group consisting of a bond, C₁₋₈ alkylene, C₂₋₈ alkenylene, arylene, and heteroarylene, and each R¹ can be independently selected from the group consisting of hydrogen, C₁₋₈ alkyl, C₂₋₈ alkenyl, C₂₋₈ alkynyl, C₃₋₈ cycloalkyl, aryl, heteroaryl, heterocyclyl, and arylalkyl. Exemplary amides include, but are not limited to, —C₁₋₈ alkylene-C(O)N(aryl)-, —C₂₋₈ alkenylene-C(O)N(aryl)-, and —C₁₋₈ alkylene-C(O)N(C₁₋₈ alkyl)- (e.g., —(CH₂)₂—C(O)N(CH₃)—).

The term “ester” means a group having the formula —C(O)O—. Esters include, e.g., —R—C(O)O—R—, —CHR¹—C(O)O—R—, and —C(R¹)(R¹)—C(O)O—R—. Each R can be independently selected from the group consisting of a bond, C₁₋ₛ alkylene, C₂₋₈ alkenylene, arylene, and heteroarylene, and each R¹ can be independently selected from the group consisting of hydrogen, C₁₋₈ alkyl, C₂₋₈ alkenyl, C₂₋₈ alkynyl, C₃₋₈ cycloalkyl, aryl, heteroaryl, heterocyclyl, and arylalkyl. Exemplary esters include, but are not limited to, —C₁₋₈ alkylene-C(O)O-arylene-, —C₂₋₈ alkenylene-C(O)O-arylene-, —C₁₋₈ alkylene-C(O)O-heteroarylene-, —C₁₋₈ alkylene-C(O)O—C₁₋₈ alkylene- (e.g., —(CH₂)₂—C(O)O—(CH₂)₂—), and —C₁₋₈ alkylene-C(O)O— (e.g., (CH₂)₂—C(O)O—).

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring.

As used herein, the term “radical” means an atom, molecule, compound, or ion that has at least one unpaired valence electron or an open electron shell.

The term “monocyclic” used herein indicates a molecular structure having one ring.

The term “polycyclic” or “multi-cyclic” used herein indicates a molecular structure having two or more rings, including, but not limited to, fused, bridged, or spiro rings.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

The term “halogen” means fluorine, chlorine, bromine, or iodine.

A “peptide” as used herein is any oligomer of two or more natural or non-natural amino acids, including alpha amino acids, beta amino acids, gamma amino acids, L-amino acids, D-amino acids, and combinations thereof. In preferred embodiments, the peptide is ˜2 to ˜30 (e.g., ˜2 to ˜5, ˜2 to ˜10, ˜5 to ˜10, ˜2 to ˜17, ˜5 to ˜17, ˜10 to ˜17, ˜5 to ˜30, ˜10 to ˜30, or ˜18 to ˜30) amino acids in length. Typically, the peptide is 10-17 amino acids in length. In at least one embodiment, the peptide contains a mixture of alpha and beta amino acids, preferably in the pattern α3/β1.

An amino acid as used herein can be any natural or non-natural amino acid, including alpha amino acids, beta amino acids, gamma amino acids, L-amino acids, and D-amino acids. Amino acid side chains can be any amino acid side chain of such an amino acid.

An amino acid according to the present invention also includes an analogue of a natural or non-natural amino acid. An amino acid analogue is an alpha amino acid with a nonnatural side chain consisting of alkyl, cycloalkyl, aryl, cycloaryl, alkenyl, or alkynyl; or a beta3-amino acid with a side chain consisting of alkyl, cycloalkyl, aryl, cycloaryl, alkenyl, or alkynyl. As used herein, an amino acid analogue also refers to a natural or nonnatural amino acid that may be substituted for an amino acid residue in the coiled-coil without loss of function relative to the native coiled-coil sequence. Suitable amino acid analogues/substitutions include the natural amino acid substitutions described in Betts & Russell, “Amino Acid Properties and Consequences of Substitutions,” in Bioinformatics for Geneticists 289-316 (Michael R. Barnes & Ian C. Gray eds. 2003), which is hereby incorporated by reference in its entirety, as well as the nonnatural substitutions described in Gfeller et al., “SwissSidechain: A Molecular and Structural Database of Non-Natural Sidechains,” Nucl. Acids Res. 41:D327-D332 (2013), which is hereby incorporated by reference in its entirety. As will be understood by the skilled artisan, analogues in the table below that are listed as having a protecting group at the N- and/or C-terminal would be deprotected during conjugation to an adjacent residue.

Unless otherwise stated, structures depicted herein are also meant to include all isomeric (e.g., enantiomeric, diastereomeric, and geometric (or conformational)) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the invention.

Unless otherwise stated, all tautomeric forms of the compounds of the invention are within the scope of the invention.

Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a ¹¹C- or ¹³C- or ¹⁴C-enriched carbon are within the scope of this invention.

It is also to be understood that the mention of one or more method steps does not preclude the presence of additional method steps or intervening method steps between those steps expressly identified. Similarly, it is also to be understood that the mention of one or more components in a device or system does not preclude the presence of additional components or intervening components between those components expressly identified.

Also, in describing the exemplary embodiments, terminology will be resorted to for the sake of clarity. It is intended that each term contemplates its broadest meaning as understood by those skilled in the art and includes all technical equivalents which operate in a similar manner to accomplish a similar purpose.

### Compounds

In one aspect, provided herein is a compound having the structure according to Formula (I):

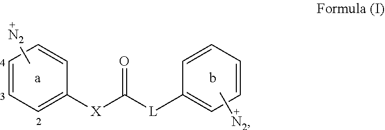

or a pharmaceutically acceptable salt thereof, wherein: X is selected from the group consisting of S, Se, P, NH, and O; and L is selected from the group consisting of S, Se, P, bond and NH; wherein: when X is NH and L is bond, N₂⁺ in ring a is not located at the 4 position. In certain embodiments, the compound is:

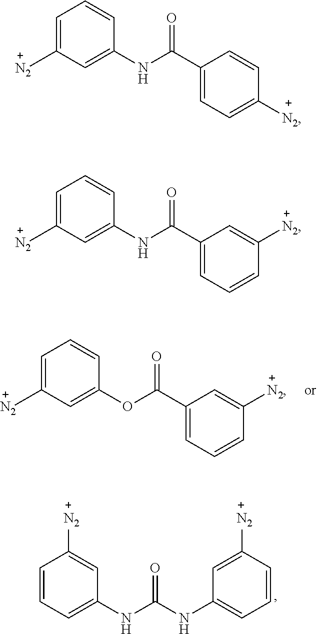

or a pharmaceutically acceptable salt thereof.

In a further aspect, provided herein is a compound having the structure according to Formula (II):

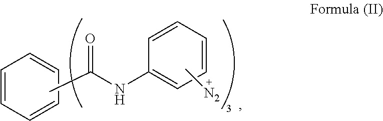

or a pharmaceutically acceptable salt thereof. In certain embodiments, the compound is

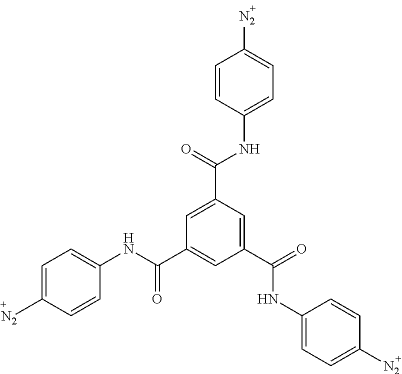

or a pharmaceutically acceptable salt thereof.

In another aspect, provided herein is a compound having the structure selected from the group consisting of Formula (III) and Formula (IV):

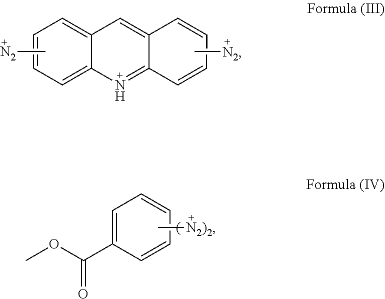

or a pharmaceutically acceptable salt thereof. In certain embodiments, the compound is:

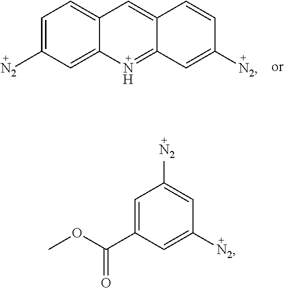

or a pharmaceutically acceptable salt thereof.

In another aspect, provided herein is a compound having the structure according to Formula (V):

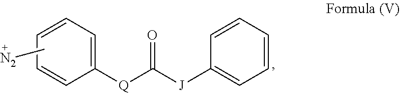

or a pharmaceutically acceptable salt thereof wherein: Q is selected from the group consisting of NH and bond; and J is selected from the group consisting of NH and bond. In certain embodiments, the compound is:

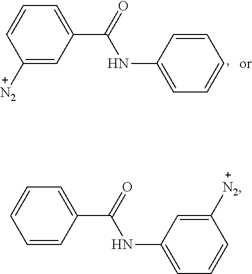

or a pharmaceutically acceptable salt thereof.

In a further aspect, provided herein is a compound having the structure according to Formula (XVII):

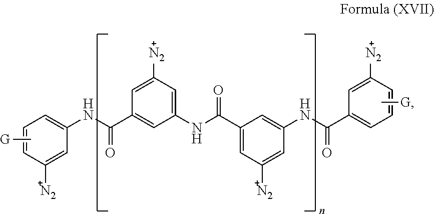

or a pharmaceutically acceptable salt thereof, wherein: G is independently at each occurrence selected from the group consisting of H, halogen, carbonyl, ester, ketone, amide, alkyl, cycloalkyl, aryl, arylalkyl, alkenyl, heteroalkyl, cyano, alkoxy, amine, haloalkyl, heteroatoms, and heterocycle; and n is selected from the group consisting of 1, 2, 3, 4, 5, 6, 7, 8, 9, and 10. In certain embodiments, the compound is

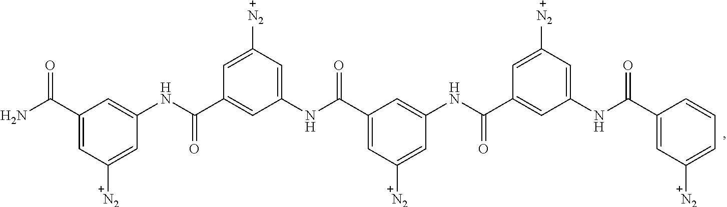

or a pharmaceutically acceptable salt thereof.

In another aspect, provided herein is a compound having the structure selected from the group consisting of Formula (XVIII), Formula (XIX), Formula (XX), Formula (XXI), Formula (XXII), and Formula (XXIII):

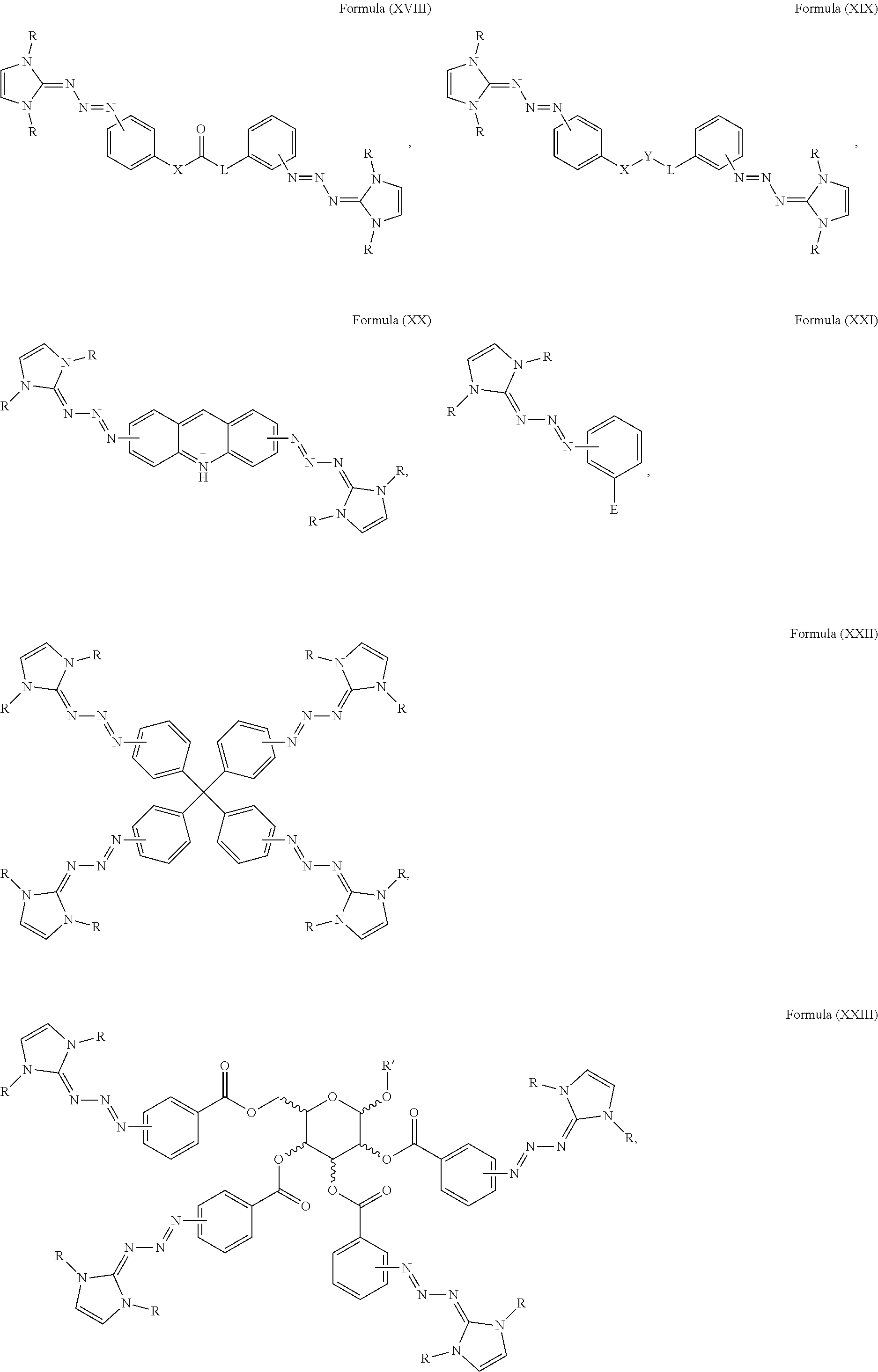

or a pharmaceutically acceptable salt thereof, wherein: R is selected from the group consisting of methyl, ethyl, isopropyl, mesityl, and alkyl sulfonate; X is selected from the group consisting of S, Se, P, bond, NH, NMe, and O; L is selected from the group consisting of S, Se, P, bond, and NH; Y is selected from the group consisting of O and C═O; E is selected from the group consisting of CN, CO₂Me, OMe, and NO₂; and R′ is selected from the group consisting of H, alkyl, cycloalkyl, halogen, alkenyl, aryl, arylalkyl, haloalkyl, heteroalkyl, acetyl, heterocycle, amine, amide, ether, and carbonyl, and any combination thereof.

In a further aspect, provided herein is a compound having the structure selected from the group consisting of Formula (XXV), Formula (XXVI), Formula (XXVII), Formula (XXVIII), Formula (XXIX), Formula (XXX), and Formula (XXXI):

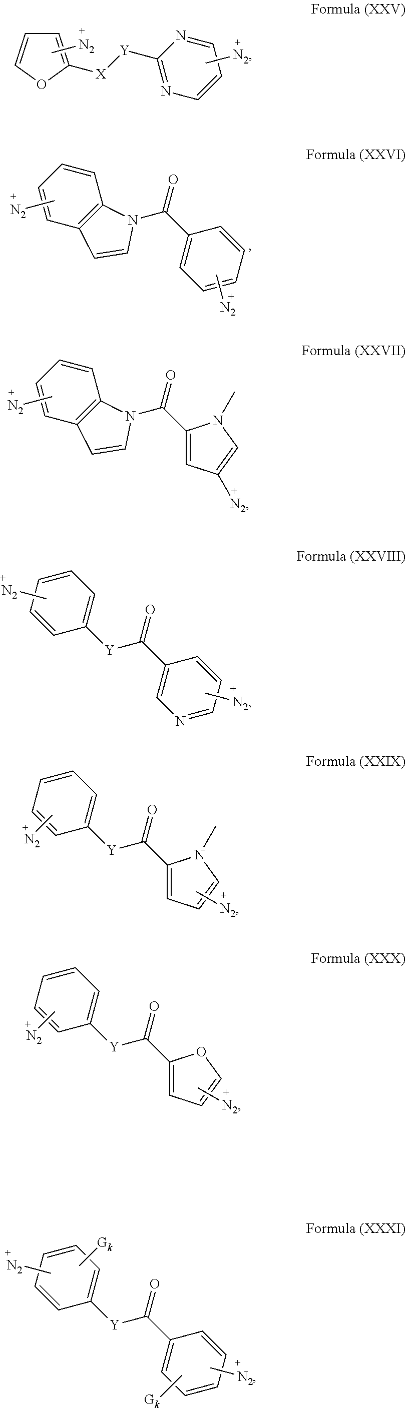

or a pharmaceutically acceptable salt thereof, wherein: X and Y are independently selected from the group consisting of bond, alkyl, S, Se, P, C═O, NH, NR′, S, O, amide, and ester; k is independently at each occurrence from 0 to 4; G is independently at each occurrence selected from the group consisting of H, halogen, carbonyl, ester, ketone, amide, alkyl, cycloalkyl, aryl, arylalkyl, alkenyl, heteroalkyl, cyano, alkoxy, amine, haloalkyl, heteroatoms, and heterocycle; and R′ is selected from the group consisting of H, alkyl, cycloalkyl, halogen, alkenyl, aryl, arylalkyl, haloalkyl, heteroalkyl, acetyl, heterocycle, amine, amide, ether, and carbonyl, and any combination thereof.

In another aspect, provided herein is a compound having the structure selected from the group consisting of Formula (I), Formula (II), Formula (III), Formula (IV), Formula (V), Formula (VI), Formula (VII), Formula (VIII), Formula (IX), Formula (X), Formula (XI), Formula (XII), Formula (XIII), Formula (XIV), Formula (XVII), Formula (XVIII), Formula (XIX), Formula (XX), Formula (XXI), Formula (XXII), Formula (XXIII), Formula (XXIV), Formula (XXV), Formula (XXVI), Formula (XXVII), Formula (XXVIII), Formula (XXIX), Formula (XXX), Formula (XXXI), and Formula (XXXII):

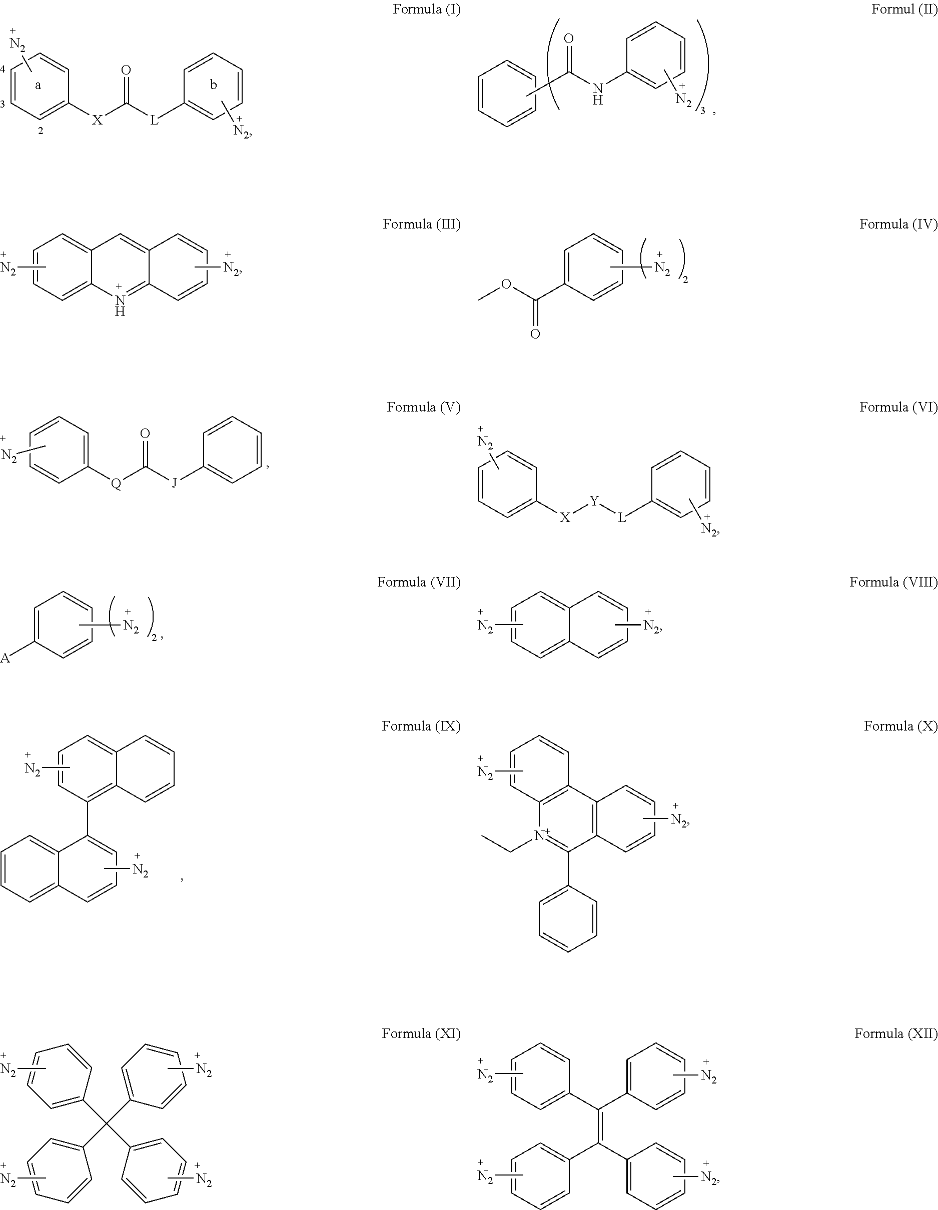

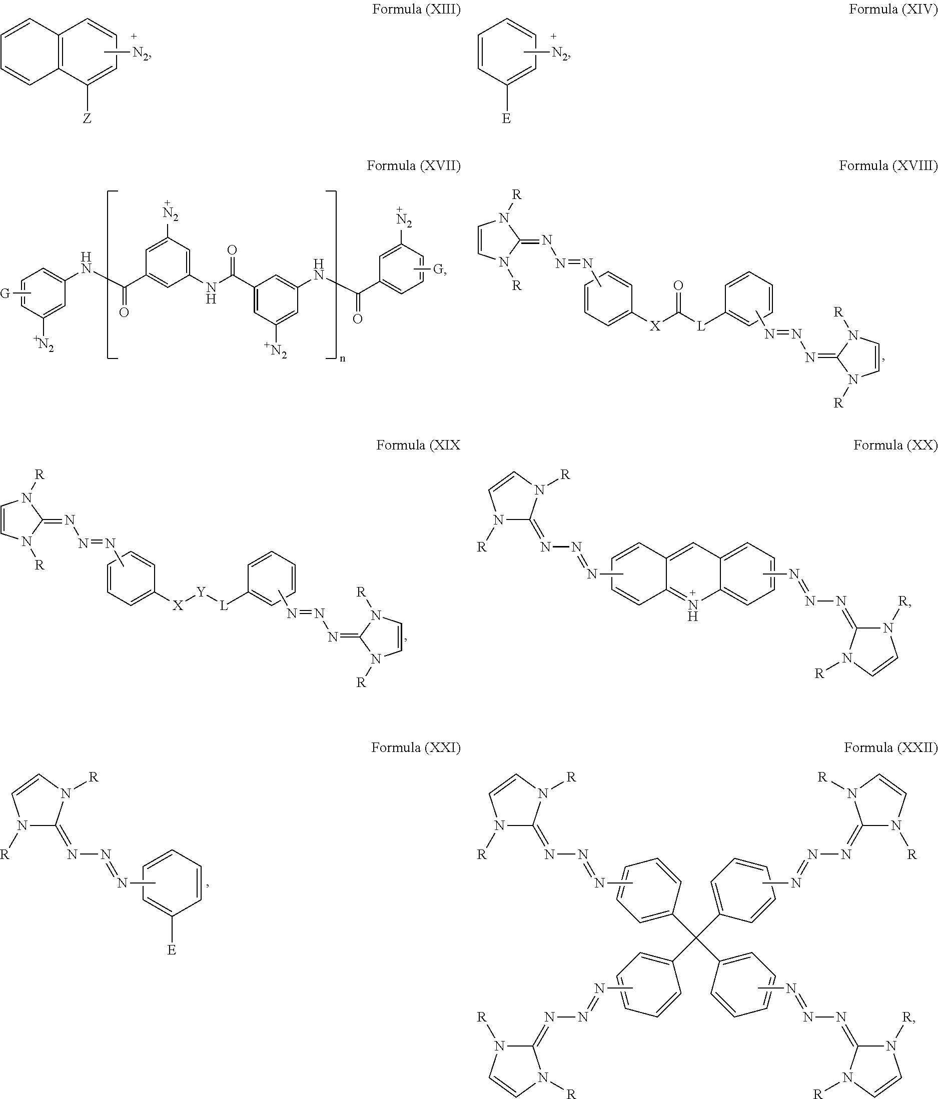

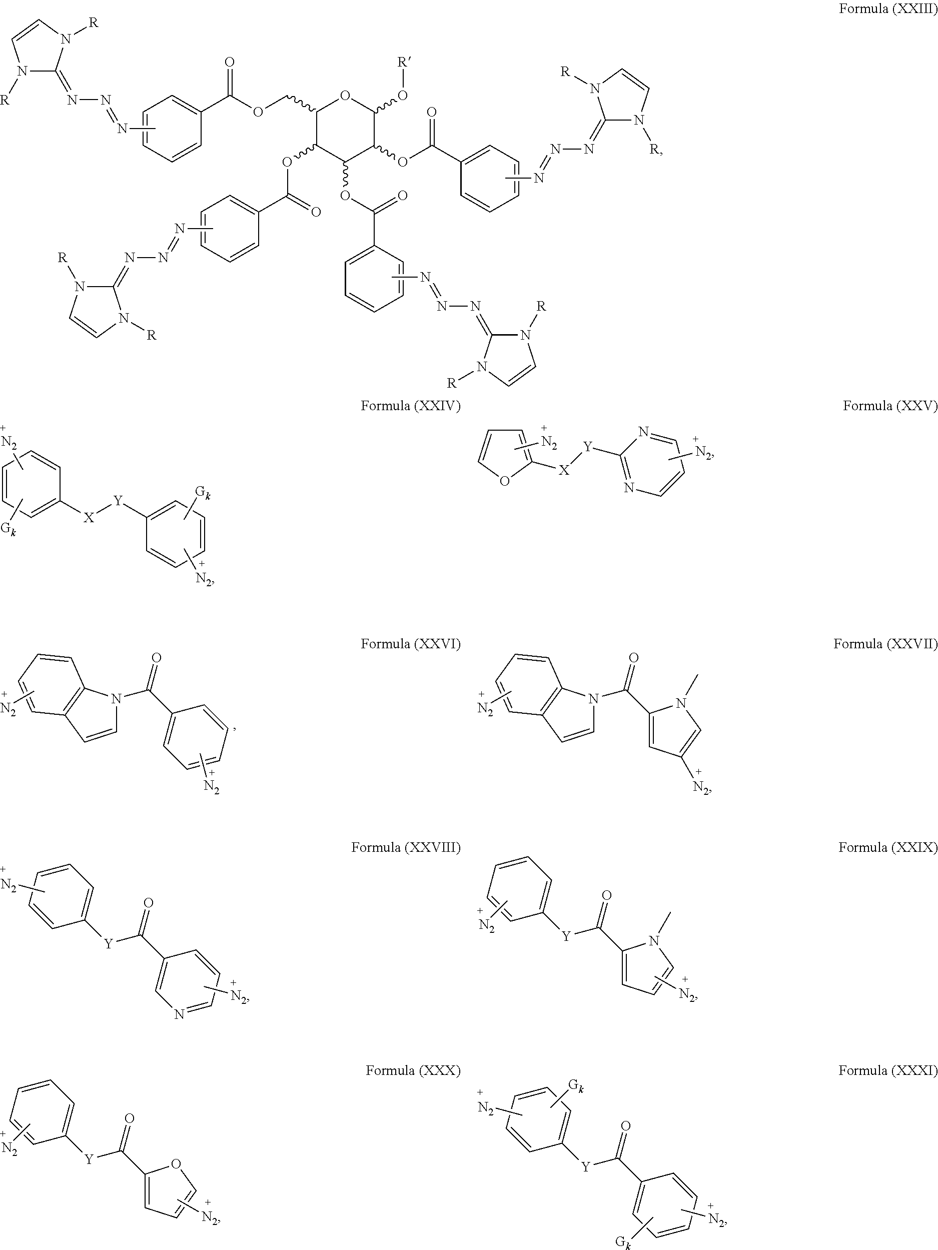

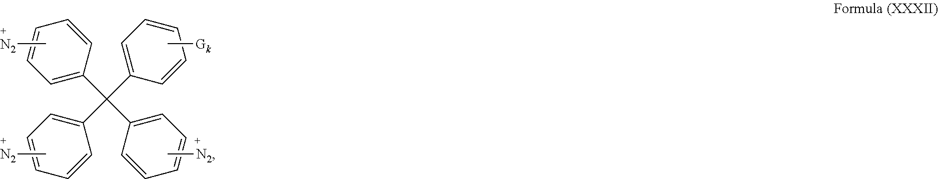

or a pharmaceutically acceptable salt thereof, wherein: X is selected from the group consisting of S, Se, P, bond, alkyl, C═O, NH, NR′, amide, ester, and O; L is selected from the group consisting of S, Se, P, bond, and NH; Q is selected from the group consisting of NH and bond; J is selected from the group consisting of NH and bond; Y is selected from the group consisting of bond, alkyl, S, Se, P, NH, NR′, S, amide, ester, 0, and C═O; A is selected from the group consisting of CO₂Me and H; Z is selected from the group consisting of H and NO₂; and E is selected from the group consisting of CN, CO₂Me, OMe, and NO₂; G is independently at each occurrence selected from the group consisting of H, halogen, carbonyl, ester, ketone, amide, alkyl, cycloalkyl, aryl, arylalkyl, alkenyl, heteroalkyl, cyano, alkoxy, amine, haloalkyl, heteroatoms, and heterocycle; n is selected from the group consisting of 1, 2, 3, 4, 5, 6, 7, 8, 9, and 10; k is independently at each occurrence from 0 to 4; R is selected from the group consisting of methyl, ethyl, isopropyl, mesityl, and alkyl sulfonate; and R′ is selected from the group consisting of H, alkyl, cycloalkyl, halogen, alkenyl, aryl, arylalkyl, haloalkyl, heteroalkyl, acetyl, heterocycle, amine, amide, ether, and carbonyl, and any combination thereof; wherein said compound, optionally in addition to more than one molecule of said compound, is conjugated to a compound selected from the group consisting of a sugar, a heterocycle, an antibody, a minor groove binder, an intercalator, an amino acid, and a peptide. In certain embodiments, the conjugation is direct. In certain embodiments, the conjugation is via a linker. In certain embodiments, the linker is selected from the group consisting of alkyl, heteroatom, heteroalkyl, haloalkyl, alkenyl, alkoxy, ester, ether, amide, hydrazine, hydrazone, acetal, ketal, disulfide, phosphate, Mal-PEG-NHS ester, DBCO-NHS ester, and SMCC.

In certain embodiments, the compound is conjugated to a sugar. In certain embodiments, the compound has the structure selected from the group consisting of Formula (XV) and Formula (XVI):

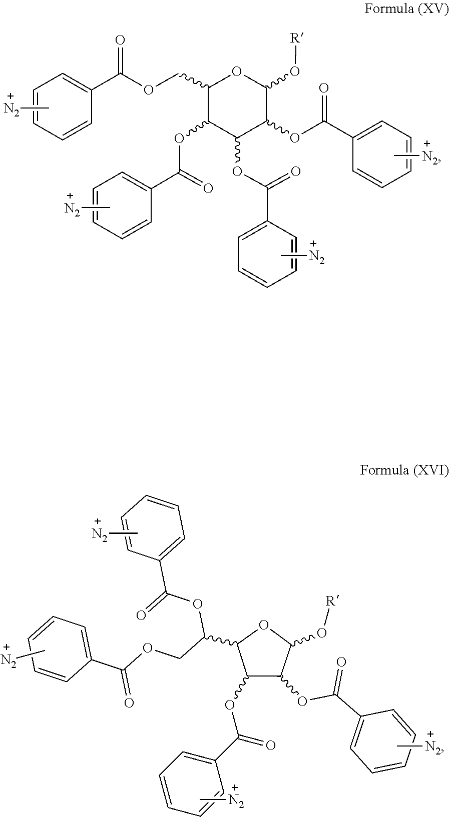

or a pharmaceutically acceptable salt thereof, wherein: R′ is selected from the group consisting of H, alkyl, cycloalkyl, halogen, alkenyl, aryl, arylalkyl, haloalkyl, heteroalkyl, acetyl, heterocycle, amine, amide, ether, carbonyl, maleimide (Mal)-polyethylene glycol (PEG)-N-Hydroxysuccinimide (NHS) ester, dibenzocyclooctyne (DBCO)—NHS ester, and succinimidyl 4-(N-maleimidomethyl)cyclohexane-1-carboxylate (SMCC), and any combination thereof. In certain embodiments, the compound is:

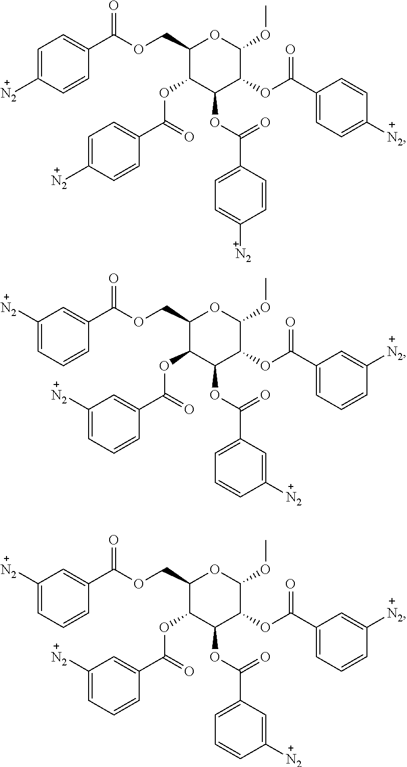

or a pharmaceutically acceptable salt thereof. In certain embodiments, the sugar is glucose, galactose, mannose, or fructose. In certain embodiments, the sugar is a pyranose. In certain embodiments, the sugar is a furanose. In certain embodiments, the sugar is a monosaccharide. In certain embodiments, the sugar is a disaccharide. In certain embodiments, the sugar is a polysaccharide.

In certain embodiments, the compound is conjugated to a heterocycle. In certain embodiments, the heterocycle is pyrrole, pyridine, indole, furan, imidazole, pyrazole, thiazole, or thiophene.

In certain embodiments, the compound is conjugated to an antibody. In certain embodiments, the antibody is an anti-CD33 antibody, an anti-CD22 antibody, or trastuzumab.

In certain embodiments, the compound is conjugated to a minor groove binder. In certain embodiments, the minor groove binder is netropsin or distamycin A.

In certain embodiments, the compound is conjugated to an intercalator. In certain embodiments, the intercalator is ethidium bromide, an anthracycline, or an acridine derivative.

In certain embodiments, the compound is conjugated to an amino acid.

In certain embodiments, the compound is conjugated to a peptide.

In certain embodiments, the compound is a salt comprising a counterion selected from the group consisting of tetrafluoroborate, hexafluorophosphate, tosylate, chloride, bromide, and hydrogen sulfate.

Compounds described herein may contain one or more asymmetric centers and may thus give rise to enantiomers, diastereomers, and other stereoisomeric forms. Each chiral center may be defined, in terms of absolute stereochemistry, as (R)- or (S)-. This technology is meant to include all such possible isomers, as well as mixtures thereof, including racemic and optically pure forms. Optically active (R)- and (S)-, (−)- and (+)-, or (D)- and (L)-isomers may be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers. Likewise, all tautomeric forms are also intended to be included.

The present invention also includes salts of the compounds described herein. As used herein, “salts” refers to derivatives of the disclosed compounds wherein the parent compound is modified by converting an existing acid or base moiety to its salt form. Examples of salts include, but are not limited to, mineral acid (such as HCl, HBr, H₂SO₄) or organic acid (such as acetic acid, benzoic acid, trifluoroacetic acid salts of basic residues such as amines; alkali (such as Li, Na, K, Mg, Ca) or organic (such as trialkylammonium) salts of acidic residues such as carboxylic acids; and the like. The salts of the present application can be synthesized from the parent compound which contains a basic or acidic moiety via conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile (ACN) are preferred.

Preparation of compounds can involve the protection and deprotection of various chemical groups. The need for protection and deprotection, and the selection of appropriate protecting groups can be readily determined by one skilled in the art. The chemistry of protecting groups can be found, for example, in Wuts and Greene, Greene Protective Groups in Organic Synthesis, 4th Ed., John Wiley & Sons: New York, 2006.

### Pharmaceutical Compositions

In certain embodiments, a pharmaceutical composition comprises the compound as described herein, or a pharmaceutically acceptable salt thereof.

In certain embodiments, a pharmaceutical dosage form comprises the compound as described herein, or the pharmaceutical composition as described herein, or a pharmaceutically acceptable salt thereof.

When employed as pharmaceuticals, the compounds of this invention are typically administered in the form of a pharmaceutical composition. Such compositions can be prepared in a manner well known in the pharmaceutical art and comprise at least one active compound.

The pharmaceutical composition can be in the form of, for example, granules, powders, tablets, capsules, syrup, suppositories, injections, emulsions, elixirs, suspensions, or solutions.

The pharmaceutical compositions of this invention can be administered by a variety of routes including oral, rectal, intraocular, transdermal, subcutaneous, intravenous, intramuscular, intraperitoneal, intradermal, directly into cerebrospinal fluid, intratracheal, and intranasal. Depending on the intended route of delivery, the compounds of this invention are preferably formulated as either injectable or oral compositions or as salves, as lotions or as patches all for transdermal administration.

The compositions for oral administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules, or the like in the case of solid compositions.

Liquid forms suitable for oral administration may include a suitable aqueous or nonaqueous vehicle with buffers, suspending and dispensing agents, colorants, flavors, and the like. Solid forms may include, for example, any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

Injectable compositions are typically based upon injectable sterile saline or phosphate-buffered saline or other injectable carriers known in the art. The active compound in such compositions is typically a minor component, often being from about 0.05 to 10% by weight with the remainder being the injectable carrier and the like.

Transdermal compositions are typically formulated as a topical ointment or cream containing the active ingredient(s), generally in an amount ranging from about 0.01 to about 20% by weight, preferably from about 0.1 to about 20% by weight, preferably from about 0.1 to about 10%) by weight, and more preferably from about 0.5 to about 15% by weight. When formulated as an ointment, the active ingredients will typically be combined with either a paraffinic or a water-miscible ointment base. Alternatively, the active ingredients may be formulated in a cream with, for example an oil-in-water cream base. Such transdermal formulations are well-known in the art and generally include additional ingredients to enhance the dermal penetration of stability of the active ingredients or the formulation. All such known transdermal formulations and ingredients are included within the scope of this invention.

The compounds of this invention can also be administered by a transdermal device. Accordingly, transdermal administration can be accomplished using a patch either of the reservoir or porous membrane type, or of a solid matrix variety.

The above-described components for orally administrable, injectable, or topically administrable compositions are merely representative. Other materials as well as processing techniques and the like are set forth in Part 8 of Remington's Pharmaceutical Sciences, 17th edition, 1985, Mack Publishing Company, Easton, Pennsylvania, which is incorporated herein by reference.

Pharmaceutical compositions containing the compounds of the invention can be prepared in combination with one or more pharmaceutically acceptable carriers. In making the compositions of the invention, the active ingredient is typically mixed with an excipient, diluted by an excipient, or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments, soft and hard gelatin capsules, suppositories, sterile injectable solutions, and sterile packaged powders.

In some embodiments, the pharmaceutical composition of the invention is in liquid form. Liquid forms include, by way of non-limiting example, emulsions, solutions, suspensions, syrups, slurries, dispersions, colloids, and the like. In some embodiments, a pharmaceutical composition described herein is in liquid, semi-solid or solid (e.g., powder) form. In specific embodiments, a pharmaceutical composition described herein is in semi-solid form, e.g., a gel, a gel matrix, a cream, a paste, or the like. In some embodiments, semi-solid forms comprise a liquid vehicle. In some embodiments, the pharmaceutical composition of the invention is a solid dosage form, such a tablet, a granule, a sachet, or a powder. Also provided are pharmaceutical compositions comprising a compound of the invention or a pharmaceutically acceptable salt thereof in the form of a dissolving tablet, a dissolving wafer, a capsule, or a gel capsule. In certain embodiments, solid dosage forms described herein comprise a solid vehicle (e.g., as used in a tablet), and/or a gaseous vehicle (e.g., as used in DPI).

In some embodiments, a composition is in a unit dose formulation for oral, intranasal, or other administration to a patient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

The active compound can be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

In some embodiments, the compounds or compositions described herein are administered intranasally. As used herein, “nasal delivery-enhancing agents” include agents which enhance the release or solubility (e.g., from a formulation delivery vehicle), diffusion rate, penetration capacity and timing, uptake, residence time, stability, effective half-life, peak or sustained concentration levels, clearance and other desired nasal delivery characteristics (e.g., as measured at the site of delivery, or at a selected target site of activity such as the brain) of the compounds or compositions of the invention. Enhancement of mucosal delivery can thus occur by any of a variety of mechanisms, for example by increasing the diffusion, transport, persistence or stability of the compounds or compositions of the invention, enzyme inhibition, increasing membrane fluidity, modulating the availability or action of calcium and other ions that regulate intracellular or paracellular permeation, solubilizing mucosal membrane components (e.g., lipids), changing non-protein and protein sulfhydryl levels in mucosal tissues, increasing water flux across the mucosal surface, modulating epithelial junctional physiology, reducing the viscosity of mucus overlying the mucosal epithelium, reducing mucociliary clearance rates, increasing nasal blood flow and other mechanisms. Suitable mucosal delivery enhancing agents will be clear to a person skilled in the art of pharmacology.

Compositions of the invention can be simple aqueous (e.g., saline) solutions. Alternatively, they can contain various additional ingredients which enhance stability and/or nasal delivery of the compounds of the invention. Such additional ingredients are well known in the art. In various embodiments of the invention, a compound of the invention is combined with one or more nasal delivery-enhancing agents. These nasal delivery-enhancing agents may be admixed, alone or together, with the nasal carrier and with the compound of the invention, or otherwise combined therewith in a pharmaceutically acceptable formulation or delivery vehicle. Furthermore, there should be no substantial, cumulative toxicity, nor any permanent deleterious changes induced in the barrier properties of the nasal mucosa with long term use. In addition to the compound of the invention, the nasal carrier and, optionally, one or more further additives and/or agents, the composition of the invention may further comprise one or more additional therapeutic ingredients (or active substances). These therapeutic ingredients can be any compound that elicits a desired activity or therapeutic or biological response in the subject.

In one aspect, a composition or unit dosage form according to the invention is formulated for sublingual administration, wherein the unit dosage form is a film including one or more disintegrants (e.g., materials that favor disintegration or fast dissolution by virtue of their solubility in water, such as hydrolyzed starches, sugars, and glycerin, which may play a dual role as a plasticizer and disintegrant) and a plasticizing agent, the film having a first portion including apomorphine hydrochloride, and a second portion including pH neutralizing agent, wherein the unit dosage form includes from 0.5 to 5 mg, from 4 to 10 mg, or from 8 to 20 mg of apomorphine hydrochloride and the pH neutralizing agent is present in an amount sufficient to produce a solution having a pH of between 3.0 and 6.0, preferably between 4.5 and 6.5, (e.g., a pH of between 2.5 and 4.5, 3.0 and 6.0, 3.5 and 6.5, 4.5 and 6.5, or 5.0 and 6.0) when the unit dosage form is placed in unbuffered water at pH 7 (e.g., the pH observed within 5 minutes of placing the unit dosage form in 1, 5, or 10 mL of unbuffered water). The film can include from 1 to 50% (w/w) (e.g., 1±0.75%, 2±1.5%, 3±0.5%, 5±2%, 7.5±2.5%, 10±2%, 14±3%, 18±4%, 22±5%, 25±5%, 30±5%, 35±5%, 40±5%, 45±5%, or 50±5% (w/w)) of the one or more disintegrants. In certain embodiments, the unit dosage form further includes a high molecular weight polymer having a weight average molecular weight of greater than 60 KDa selected from hydroxypropyl cellulose, hydroxypropyl methyl cellulose, hydroxyethyl cellulose, carboxymethyl cellulose, and methyl cellulose. In other embodiments, the unit dosage form further includes a low molecular weight polymer having a weight average molecular weight of from 5 KDa to 50 KDa selected from hydroxypropyl cellulose, hydroxypropyl methyl cellulose, hydroxyethyl cellulose, carboxymethyl cellulose, and methyl cellulose. The pH neutralizing agent can be an organic base (e.g., pyridoxine, meglumine, or any organic base described herein) or an inorganic base (e.g., magnesium hydroxide, sodium bicarbonate, or an inorganic base described herein). In particular embodiments, the unit dosage form includes 35±5% (w/w) disintegrant, from 0.5 to 5 mg, from 4 to 10 mg, or from 8 to 20 mg of apomorphine hydrochloride and pyridoxine present in an amount sufficient to produce a solution having a pH of between 4.5 and 6.5 when the unit dosage form is placed in unbuffered water at pH 7. Suitable film for oral administration of the compositions according to the invention is disclosed in, e.g., U.S. Pat. No. 8,846,074.

In some embodiments, a composition or unit dosage form described herein is administered as an emulsion, a solution, a suspension, a syrup, a slurry, a dispersion, a colloid, a dissolving tablet, a dissolving wafer, a capsule, a gel capsule, a semi-solid, a solid forma gel, a gel matrix, a cream, a paste, a tablet, a granule, a sachet, a powder, or the like. In certain aspects, about 0.000001 mg to about 2000 mg, about 0.00001 mg to about 1000 mg, or about 0.0001 mg to about 750 mg, about 0.001 mg to about 500 mg, about 0.01 mg to about 250 mg, about 0.1 mg to about 100 mg, about 0.5 mg to about 75 mg, about 1 mg to about 50 mg, about 2 mg to about 40 mg, about 5 mg to about 20 mg, or about 7.5 mg to about 15 mg of compound of any one of the formulas described herein per day or per dose is administered to an individual.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g., about 40 mesh. Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the invention can be formulated so as to provide quick, sustained, or delayed release of the active ingredient after administration to the patient by employing procedures known in the art.

For preparing solid compositions such as tablets, the principal active ingredient is mixed with a pharmaceutical excipient to form a solid pre-formulation composition containing a homogeneous mixture of the compound of any formula as described herein. When referring to these pre-formulation compositions as homogeneous, the active ingredient is typically dispersed evenly throughout the composition so that the composition can be readily subdivided into equally effective unit dosage forms such as tablets, pills, and capsules. This solid pre-formulation is then subdivided into unit dosage forms of the type described above containing from, for example, 0.000001 to about 2000 mg of the active ingredient of the present application.

The tablets or pills containing the compound of any of the formulas as described herein can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the compounds and compositions of the present application can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous, or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions in can be nebulized by use of inert gases. Nebulized solutions may be breathed directly from the nebulizing device or the nebulizing device can be attached to a face masks tent, or intermittent positive pressure breathing machine. Solution, suspension, or powder compositions can be administered orally or nasally from devices which deliver the formulation in an appropriate manner.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9. It will be understood that use of certain of the foregoing excipients, carriers, or stabilizers will result in the formation of pharmaceutical salts.

The therapeutic dosage of the compounds of the invention can vary according to, for example, the particular use for which the treatment is made, the manner of administration of the compound, the health and condition of the patient, and the judgment of the prescribing physician. The proportion or concentration of the compounds of the invention in a pharmaceutical composition can vary depending upon a number of factors including dosage, chemical characteristics (e.g., hydrophobicity), and the route of administration. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

### Methods of Use

In certain embodiments, a method of cleaving DNA in a cell comprises contacting the cell with an effective amount of the compound as described herein, the pharmaceutical composition as described herein, or the pharmaceutical dosage as described herein.

In another aspect, provided herein is a method of cleaving DNA in a cell comprising contacting the cell with an effective amount of a compound according to Formula (XXIV):

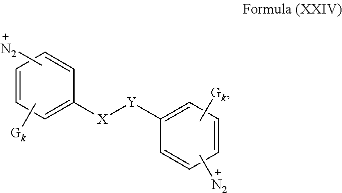

or a pharmaceutically acceptable sale thereof, wherein: X and Y are independently selected from the group consisting of bond, alkyl, S, Se, P, C═O, NH, NR′, S, O, amide, and ester; k is independently at each occurrence from 0 to 4; G is independently at each occurrence selected from the group consisting of H, halogen, carbonyl, ester, ketone, amide, alkyl, cycloalkyl, aryl, arylalkyl, alkenyl, heteroalkyl, cyano, alkoxy, amine, haloalkyl, heteroatoms, and heterocycle; R′ is selected from the group consisting of H, alkyl, cycloalkyl, halogen, alkenyl, aryl, arylalkyl, haloalkyl, heteroalkyl, acetyl, heterocycle, amine, amide, ether, and carbonyl, and any combination thereof.

In a further aspect, provided herein is a method of cleaving DNA in a cell comprising contacting the cell with an effective amount of a compound according to Formula (VI):

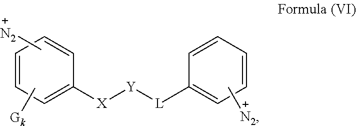

or a pharmaceutically acceptable salt thereof, wherein: X is selected from the group consisting of S, Se, P, bond, NH, NMe, and O; Y is selected from the group consisting of O and C═O; and L is selected from the group consisting of S, Se, P, bond, and NH. In certain embodiments, the compound is:

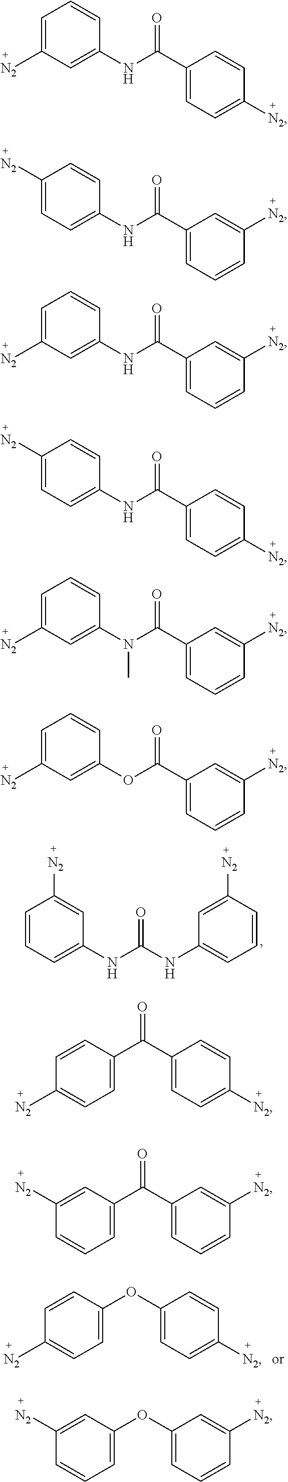

or a pharmaceutically acceptable salt thereof.

In another aspect, provided herein is a method of cleaving DNA in a cell comprising contacting the cell with an effective amount of a compound according to Formula (VII):

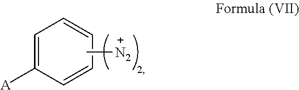

or a pharmaceutically acceptable salt thereof, wherein: A is selected from the group consisting of CO₂Me and H. In certain embodiments, the compound is:

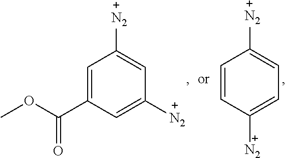

or a pharmaceutically acceptable salt thereof.

In a further aspect, provided herein is a method of cleaving DNA in a cell comprising contacting the cell with an effective amount of a compound selected from the group consisting of Formula (VIII), Formula (IX), Formula (X), Formula (XI), Formula (XII), and Formula (XXXII):

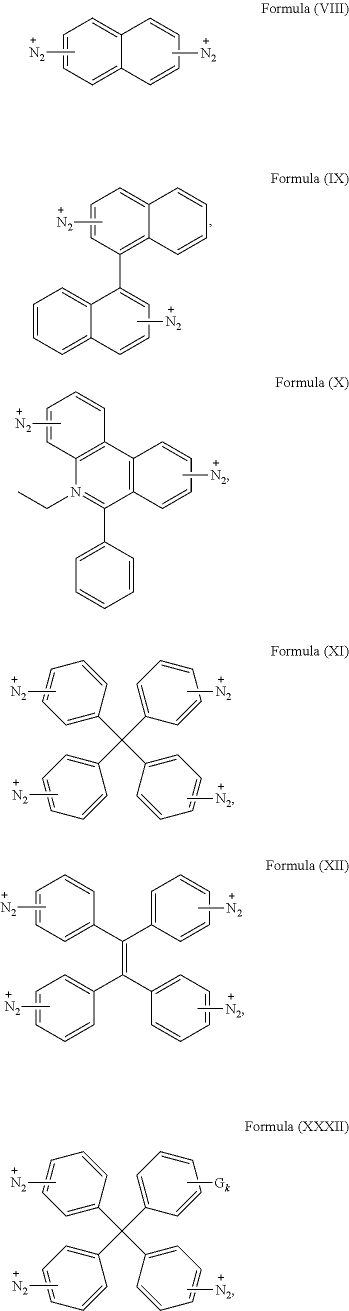

or a pharmaceutically acceptable salt thereof, wherein: k is independently at each occurrence from 0 to 4; G is independently at each occurrence selected from the group consisting of H, halogen, carbonyl, ester, ketone, amide, alkyl, cycloalkyl, aryl, arylalkyl, alkenyl, heteroalkyl, cyano, alkoxy, amine, haloalkyl, heteroatoms, and heterocycle. In certain embodiments, the compound is:

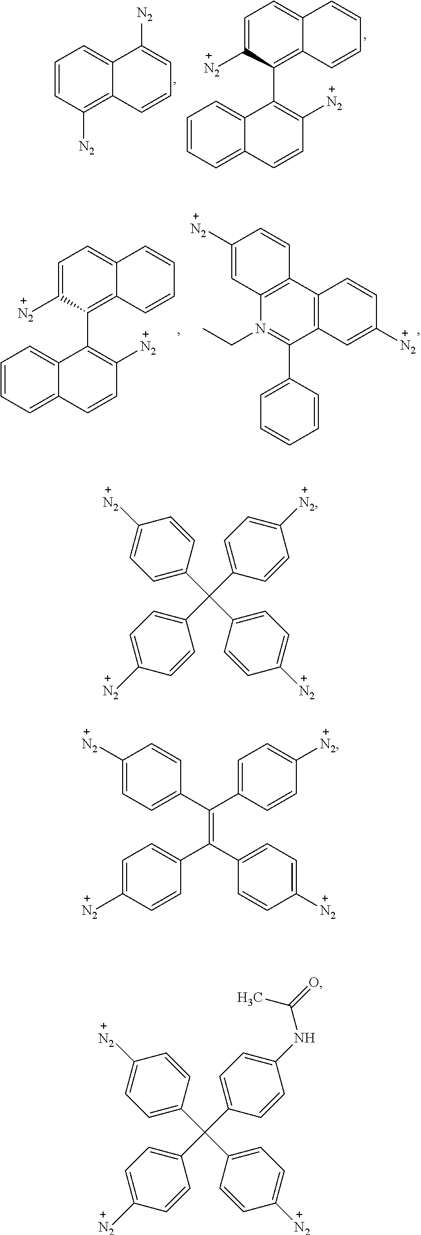

or a pharmaceutically acceptable salt thereof.

In another aspect, provided herein is a method of cleaving DNA in a cell comprising contacting the cell with an effective amount of a compound according to Formula (XIII):

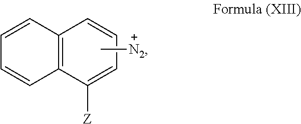

or a pharmaceutically acceptable salt thereof, wherein: Z is selected from the group consisting of H and NO₂. In certain embodiments, the compound is:

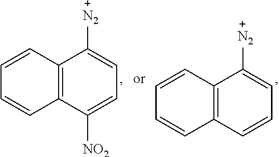

or a pharmaceutically acceptable salt thereof.

In a further aspect, provided herein is a method of cleaving DNA in a cell comprising contacting the cell with an effective amount of a compound according to Formula (XIV):

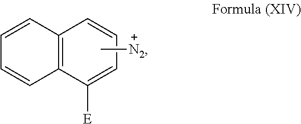

or a pharmaceutically acceptable salt thereof, wherein: E is selected from the group consisting of CN, CO₂Me, OMe, and NO₂. In certain embodiments, the compound is:

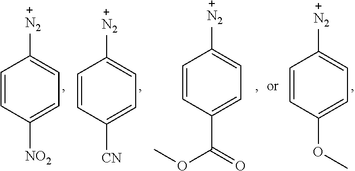

or a pharmaceutically acceptable salt thereof.

In certain embodiments, the compound is a salt comprising a counterion that is tetrafluoroborate. Other counterions may include, but are not limited to, hexafluorophosphate, perchlorate, tetrakis[3,5-bis(trifluoromethyl)phenyl]borate, tetrakis(pentafluorophenyl)borate, tris(pentafluorophenyl)boron, and perfluoroalkoxyaluminate.

In certain embodiments, the method further comprises a reduction step after contacting the cell with the compound. In certain embodiments, the reduction step comprises reduction of the compound to its corresponding radical upon nitrogen extrusion. Without wishing to be limited by theory, reduction involves the transfer of electrons between chemical species in a reaction, specifically the gain of electrons, gain of hydrogen, or loss of oxygen.

In certain embodiments, the reduction step comprises light irradiation. In certain embodiments, the light irradiation is performed with light of a wavelength comprising one or more wavelengths within the full spectrum of light wavelengths. In certain embodiments, the light irradiation is performed with light selected from the group consisting of ambient light, blue light at about 467 nm, green light at about 525 nm, red light at about 660 nm, and near-IR light at about 1040 nm to about 2500 nm. In certain embodiments, the reduction step is performed with spatial and temporal selectivity.

Light irradiation may be performed within the visible light spectrum, from about 380 nm to about 750 nm. Light irradiation may be performed within the near-IR light spectrum, from about 760 nm to about 2500 nm. Light irradiation may be performed at about 380 nm, 390 nm, 400 nm, 410 nm, 420 nm, 430 nm, 440 nm, 450 nm, 460 nm, 470 nm, 480 nm, 490 nm, 500 nm, 505 nm, 510 nm, 515 nm, 520 nm, 525 nm, 530 nm, 535 nm, 540 nm, 545 nm, 550 nm, 555 nm, 560 nm, 565 nm, 570 nm, 575 nm, 580 nm, 585 nm, 590 nm, 595 nm, 600 nm, 610 nm, 620 nm, 630 nm, 640 nm, 650 nm, 660 nm, 670 nm, 680 nm, 690 nm, 700 nm, 710 nm, 720 nm, 730 nm, 740 nm, 750 nm, 760 nm, 770 nm, 780 nm, 790 nm, 800 nm, 810 nm, 820 nm, 830 nm, 840 nm, 850 nm, 875 nm, 900 nm, 925 nm, 950 nm, 975 nm, 1000 nm, 1100 nm, 1200 nm, 1300 nm, 1400 nm, 1500 nm, 1600 nm, 1700 nm, 1800 nm, 1900 nm, 2000 nm, 2100 nm, 2200 nm, 2300 nm, 2400 nm, or 2500 nm, or any combinations thereof.

In certain embodiments, the reduction step comprises treatment of the compound with a reducing agent. In certain embodiments, the reducing agent is selected from the group consisting of NADPH, NADH, FADH₂, glutathione, cysteine, and amine reductants. In certain embodiments, the reducing agent is cysteine. In certain embodiments, the reducing agent is endogenous. In certain embodiments, the reducing agent is exogenous.

In certain embodiments, the reduction step comprises treatment of the compound with a metal reductant. In certain embodiments, the metal reductant is selected from the group consisting of copper (I) chloride, iron (II) sulfate, zinc metal, and iron metal.

In certain embodiments, the DNA cleavage comprises one or more double strand breaks in the DNA. In certain embodiments, the DNA cleavage comprises one or more single strand breaks in the DNA. In certain embodiments, the DNA comprises supercoiled double stranded DNA. In certain embodiments, the DNA comprises linear double stranded DNA.

In certain embodiments, the compound cleaves DNA with an EC₅₀ of less than 600 nM. In certain embodiments, the compound cleaves DNA with an EC₅₀ of less than 150 nM. In certain embodiments, the compound cleaves DNA with an EC₅₀ of less than 75 nM. In certain embodiments, the compound cleaves DNA with an EC₅₀ of less than 25 nM. In certain embodiments, the compound cleaves DNA with an EC₅₀ of less than 15 nM. In certain embodiments, the compound cleaves DNA with an EC₅₀ of less than 10 nM. In certain embodiments, the compound cleaves DNA with an EC₅₀ of less than 5 nM. In certain embodiments, the compound cleaves DNA with an EC₅₀ of less than 3 nM.

The compound may cleave DNA with an EC₅₀ of about 1 nM, 2 nM, 3 nM, 4 nM, 5 nM, 6 nM, 7 nM, 8 nM, 9 nM, 10 nM, 11 nM, 12 nM, 13 nM, 14 nM, 15 nM, 16 nM, 17 nM, 18 nM, 19 nM, 20 nM, 21 nM, 22 nM, 23 nM, 24 nM, 25 nM, 27 nM, 30 nM, 33 nM, 35 nM, 37 nM, 40 nM, 45 nM, 50 nM, 55 nM, 60 nM, 65 nM, 70 nM, 75 nM, 80 nM, 85 nM, 90 nM, 95 nM, 100 nM, 110 nM, 120 nM, 130 nM, 140 nM, 150 nM, 160 nM, 170 nM, 180 nM, 190 nM, 200 nM, 225 nM, 250 nM, 275 nM, 300 nM, 325 nM, 350 nM, 375 nM, 400 nM, 450 nM, 500 nM, 550 nM, 600 nM, 650 nM, 700 nM, 800 nM, 900 nM, 1000 nM, 1100 nM, 1200 nM, 1300 nM, 1400 nM, 1500 nM, 1600 nM, 1700 nM, 1800 nM, 1900 nM, 2000 nM, 2500 nM, 3000 nM, 3500 nM, 4000 nM, 4500 nM, or 5000 nM.

In certain embodiments, the cell is a bacterial cell. In certain embodiments, the cell is a mammalian cell. In certain embodiments, the mammalian cell is a tumor cell.

In a further aspect, provided herein is a method of treating a bacterial infection in a subject in need thereof, comprising administering to the subject an effective amount of the compound as described herein, the pharmaceutical composition as described herein, or the pharmaceutical dosage as described herein. In certain embodiments, the method of treating a bacterial infection in a subject in need thereof, comprises administering to the subject an effective amount of a compound selected from the group consisting of Formula (I), Formula (II), Formula (III), Formula (IV), Formula (V), Formula (VI), Formula (VII), Formula (VIII), Formula (IX), Formula (X), Formula (XI), Formula (XII), Formula (XIII), Formula (XIV), Formula (XV), Formula (XVI), Formula (XVII), Formula (XVIII), Formula (XIX), Formula (XX), Formula (XXI), Formula (XXII), Formula (XXIII), Formula (XXIV), Formula (XXV), Formula (XXVI), Formula (XXVII), Formula (XXVIII), Formula (XXIX), Formula (XXX), Formula (XXXI), and Formula (XXXII).

In another aspect, provided herein is a method of treating a viral infection in a subject in need thereof, comprising administering to the subject an effective amount of the compound as described herein, the pharmaceutical composition as described herein, or the pharmaceutical dosage as described herein. In certain embodiments, the method of treating a viral infection in a subject in need thereof, comprises administering to the subject an effective amount of a compound selected from the group consisting of Formula (I), Formula (II), Formula (III), Formula (IV), Formula (V), Formula (VI), Formula (VII), Formula (VIII), Formula (IX), Formula (X), Formula (XI), Formula (XII), Formula (XIII), Formula (XIV), Formula (XV), Formula (XVI), Formula (XVII), Formula (XVIII), Formula (XIX), Formula (XX), Formula (XXI), Formula (XXII), Formula (XXIII), Formula (XXIV), Formula (XXV), Formula (XXVI), Formula (XXVII), Formula (XXVIII), Formula (XXIX), Formula (XXX), Formula (XXXI), and Formula (XXXII).

In a further aspect, provided herein is a method of treating a cancer in a subject in need thereof, comprising administering to the subject an effective amount of the compound as described herein, the pharmaceutical composition as described herein, or the pharmaceutical dosage as described herein. In certain embodiments, the method of treating a cancer in a subject in need thereof, comprises administering to the subject an effective amount of a compound selected from the group consisting of Formula (I), Formula (II), Formula (III), Formula (IV), Formula (V), Formula (VI), Formula (VII), Formula (VIII), Formula (IX), Formula (X), Formula (XI), Formula (XII), Formula (XIII), Formula (XIV), Formula (XV), Formula (XVI), Formula (XVII), Formula (XVIII), Formula (XIX), Formula (XX), Formula (XXI), Formula (XXII), Formula (XXIII), Formula (XXIV), Formula (XXV), Formula (XXVI), Formula (XXVII), Formula (XXVIII), Formula (XXIX), Formula (XXX), Formula (XXXI), and Formula (XXXII). In certain embodiments, the cancer is a solid tumor. In certain embodiments, the cancer is selected from the group consisting of kidney cancer, adrenal gland cancer, anal cancer, appendiceal cancer, ovarian cancer, uterine cancer, gastric cancer, breast cancer, lung cancer, bladder cancer, cervical cancer, stomach cancer, sarcoma cancer, liver cancer, esophageal cancer, laryngeal cancer, multiple myeloma, colorectal cancer, rectal cancer, skin cancer, pancreatic cancer, brain or spinal cord cancer, leukemia or lymphoma. In certain embodiments, the cancer is breast cancer or human lung carcinoma.

In certain embodiments, the subject is a human.

In certain embodiments, the compound is administered to the subject orally, topically, intranasally, intravenously, intramuscularly, or subcutaneously.

Generally, the compounds of this invention are administered in a therapeutically effective amount. The amount of the compound actually administered will typically be determined by a physician, in the light of the relevant circumstances, including the condition to be treated, the chosen route of administration, the actual compound administered, the age, weight, and response of the individual patient, the severity of the patient's symptoms, and the like.

The compounds of this invention can also be administered in sustained release forms or from sustained release drug delivery systems. A description of representative sustained release materials can be found in Remington's Pharmaceutical Sciences.

The compounds of this invention can be administered as the sole active agent or they can be administered in combination with other agents, including other compounds that demonstrate the same or a similar therapeutic activity and are determined to safe and efficacious for such combined administration.

### EXAMPLES

The following examples are provided to further describe some of the embodiments disclosed herein. The examples are intended to illustrate, not to limit, the disclosed embodiments.

### General Methods

All chemical reagents were purchased from commercial suppliers (Ambeed, Oakwood Chemical, Sigma-Aldrich, TCI, Alfa, or Thermo Fisher Scientific). Agarose was sourced from Sigma-Aldrich and GelRed® DNA gel stain (10,000× in water) from Biotium. 50× Tris-acetate-EDTA (TAE) buffer was purchased from Thermo Fisher Scientific. Supercoiled pBR322 plasmid was purchased from New England Biolabs. Double distilled water was produced by ELGA Purelab Flex Water Purification System. ¹H NMR spectra were recorded on a Bruker 400 MHz and 500 MHz Avance spectrometer. Chemical shifts were reported in ppm relative to tetramethylsilane, with the residual solvent resonance (CD₃CN, δ=1.94 ppm) as the internal reference. Spectra were reported as follows: chemical shift (6 ppm), multiplicity (s=singlet, d=doublet, t=triplet, q=quartet, m=multiplet), coupling constant (Hz), and integration. ¹³C NMR spectra were recorded on Bruker 500 (126 MHz). Chemical shifts were reported in ppm relative to tetramethylsilane with the solvent resonance used as the internal reference (CD₃CN, 6=118.26 ppm). ¹⁹F NMR spectra were recorded on Bruker 500 (471 MHz). High resolution mass spectra (HRMS) were recorded on an Agilent 6224 TOF LC/MS (APCI source and ESI source). Infrared (IR) spectra were acquired using Nicolet 6700 FT-IR spectrometer through attenuated total reflectance (ATR). UV-Vis spectra were obtained using a Cary 100 UV-Visible Spectrophotometer. Substrates were synthesized according to literature and modified procedures. DNA gel electrophoresis were performed with Bio-Rad Wide Mini-Sub Cell GT Cell System. Agarose gels were pre-stained with GelRed (Biotium) and imaged by Bio-Rad ChemiDoc Imaging System.

Examination of DNA-cleaving ability of diazonium salts: Before the reaction, a fresh 500 μM stock solution of the diazonium salt was prepared by dissolving 10 μmol of the salt in 20 mL of ddH₂O. In 1.5 mL Eppendorf tubes, a serial dilution was performed to achieve a concentration range between 1000 nM and 1 nM solutions of the diazonium salts in ddH2O and 4,000 nM, 2,000 nM, 1,000 nM, 500 nM, 400 nM, 200 nM, 100 nM and 50 nM solutions of the monodiazonium salts. However, monodiazonium salt benzenediazonium 1 was diluted to concentrations of 80 μM, 40 μM, 30 μM, 20 μM, 10 μM, 4 μM, and 0.5 μM and 4-methoxy-benzene-1-diazonium 5 was diluted to concentrations of 250 μM, 100 μM, 80 μM, 60 μM, 40 μM, 10 μM, and 5 μM instead. In a standard reaction, 1 μL (0.5 μg) of aqueous supercoiled pBR322 plasmid DNA (0.5 μg/mL aqueous stock) and 20 μL of the corresponding diazonium solutions were mixed. The reaction mixtures were incubated at 25° C. with 525 nm green light irradiation (in between two Kessil pro160 green LED lights, 44 W) for 1.5-2 h. The DNA-cleaving properties of the diazonium salts were then examined by agarose gel electrophoresis. The cleavage assays were performed in duplicates. As a control within every assay, a sample of DNA with 20 μL of ddH₂O in the absence of diazonium was also irradiated along with the rest of the samples and used as an internal standard for the starting percentage of supercoiled plasmid and ensured any cleavage observed was not from irradiation or other factors during the reaction.

Agarose gel electrophoresis: The agarose gel was prepared by heating a suspension of 0.08%-1% (w/v) agarose in Tris-acetate-EDTA (TAE) buffer in a microwave until fully dissolved. GelRed® 10,000× (Biotium) was added to the solution and the stained molten suspension was then poured into a cast and allowed to solidify at room temperature for 45 minutes-1 h. DNA reaction samples were mixed with 4 μL of Gel Loading Dye Purple 6× (New England Biolabs) and loaded into the wells of the agarose gel. The gel was run at 90V in TAE buffer for 2 h at room temperature. After electrophoresis, the gel was analysed using a UV tray in a Bio-Rad ChemiDoc Imaging System. The different DNA bands were quantified with Bio-Rad Image Lab and plotted against the corresponding diazonium salt concentration in a dose-response plot to generate the EC₅₀ value.

### DNA Cleavage Assay Controls and Restriction Enzyme Calibrations:

Nb.BtsI (Nicking) and EcoRV-HF (Linearizing) Digestion of pBR322: Into two 1.5 mL Eppendorf tubes was added 0.5 μg of pBR322 (1 μL from stock of 0.5 μg/μL in each tube), 16 μL of ddH₂O (to reach total 20 μL reaction volume), and 2 μL buffer (10× CutSmart Buffer, NEB Labs). In one tube was added 1 μL Nb.BtsI (10,000 units/mL, NEB Labs) and in the second tube added 1 μL EcoRV-HF (100,000 units/mL, NEB Labs). The tubes were placed in a 37° C. incubator and allowed to incubate for 2 hours. The DNA was then examined by the standard agarose gel electrophoresis.

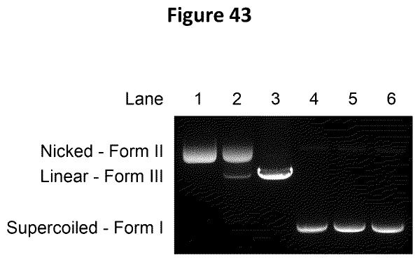

1,5-diaminonaphthalene and diazonium compound 10 digestion of pBR322: Into two 0.2 mL PCR tubes was added 0.5 μg of pBR322 (1 μL from stock of 0.5 μg/μL in each tube). Into one of the PCR tubes was added 20 μL of a 500 nM working solution of diazonium compound 10 in ddH₂O (prepared from fresh 500 μM stock solution in ddH₂O) and into the other PCR tube was added 20 μL of a 500 nM working solution of the corresponding amine precursor, 1,5-diaminonaphthalene, in ddH₂O (prepared from a fresh 500 μM stock solution in DMSO). Into a third PCR tube was added 0.5 μg of pBR322 (1 μL from stock of 0.5 μg/μL in each tube) and 20 μL ddH₂O in the absence of any additional compound. The samples were incubated together at 25° C. in between two Kessil pro160 green (525 nm) LED lights (44 W) for 1.5 hours. The DNA-cleavage of the diazonium, amine, and water controls were then examined by the standard agarose gel electrophoresis (FIG. 43).

The cleavage pattern of pBR322 (Form I) in the presence of diazonium compound 10 (500 nM) lead to two new bands (Lane 2), which are assigned as the nicked (Form II) and linear (Form III) strands. These two bands were confirmed as such by their alignment in the same gel with the matching bands formed by pBR322 incubated with the known nicking plasmid, Nb.BtsI, leading to nicked DNA (Lane 1) and pBR322 incubated with restriction enzyme EcoRV-HF, leading to linear DNA (Lane 3), respectively. Additionally, when pBR322 was irradiated with the same concentration (500 nM) of 1,5-diaminonaphthalene, the amine precursor of the diazonium, or with water alone no cleavage was observed (Lanes 4 and 5, respectively). This confirmed that in instances when pBR322 cleavage is observed, it is not from light irradiation nor the general presence of an organic small molecule.

DNA Cleavage by Irradiation with Different Wavelengths and in the Dark:

Cleavage Assay of Diazonium in Blue, Red, and Ambient light: Similar to the standard assay, a fresh 500 μM stock solution of diazonium salt compound 28 was prepared by dissolving 10 μmol (7.8 mg) of the salt in 20 mL of ddH₂O. In 1.5 mL Eppendorf tubes, a serial dilution was performed to achieve a concentration range between 1000 nM and 1 nM solutions of the diazonium salt in ddH₂O. In 0.2 mL PCR tubes, 1 μL (0.5 μg) of aqueous supercoiled pBR322 plasmid DNA (0.5 μg/μl aqueous stock) and 20 μL of the corresponding diazonium solutions were mixed. The samples were incubated at 25° C. in between either two Kessil pro160 blue (467 nm) LED lights (34 W), two Kessil pro160 red (660 nm) LED lights (35 W), or with ambient light for 1.5 hours (for ambient light reactions, the tubes were left on the bench countertop). The DNA-cleaving properties of the diazonium salts were then examined by the standard agarose gel electrophoresis procedure. The cleavage assays were performed in duplicates.

Experimental Details for the Cleavage Assay of Diazonium in the Dark: Similar to the standard assay, a fresh 500 μM stock solution of diazonium salt 28 was prepared by dissolving 10 μmol (7.8 mg) of the salt in 20 mL of ddH₂O. In a dark room, a serial dilution in 1.5 mL Eppendorf tubes was performed to achieve a range between 50,000 nM and 2.5 nM solutions of the diazonium salt in ddH₂O. In 0.2 mL PCR tubes, 1 μL (0.5 μg) of aqueous supercoiled pBR322 plasmid DNA (0.5 μg/μl aqueous stock) and 20 μL of the corresponding diazonium solutions were mixed. The samples were incubated at room temperature wrapped in aluminum foil in the dark room for 1.5 hours. The DNA-cleaving properties of the diazonium salts were then examined by the standard agarose gel electrophoresis procedure. The cleavage assay was performed in duplicates.

Digestion of pBR322: Into 8×1.5 mL Eppendorf tubes was added 5 μg of pBR322 (5 μL from stock of 1 μg/μL in each tube), 165 μL of molecular grade water (to reach total 200 μL reaction volume), 20 μL buffer (10× CutSmart Buffer, NEB Labs) and 5 μL EcoRV-HF (100,000 units/mL, NEB Labs). The tubes were placed in a 37° C. incubator and allowed to incubate for 2.5 hours. A 0.8% gel with two rows of wells was cast (large gel cast with 180 mL of molten agarose solution stained with 18 μL of 10,000× GelRed). The eight digestive reactions were combined (between the eight tubes, a total of 40 μg of pBR322 was digested). 320 μL of Gel Loading Dye Purple 6× (New England Biolabs) was added to the combined sample and the solution was loaded across the individual wells on the gel. The gel was run at 100 V in 1×TAE buffer for 1.5 hours. After electrophoresis, the gel was analyzed using a UV tray in a Bio-Rad ChemiDoc Imaging System to confirm the complete linearization of the DNA. The DNA fragments were cut out from the gel and purified with a QIAquick Gel Extraction Kit (Qiagen). After the purification between two QIAprep® 2.0 spin columns was performed, the DNA was eluted from each column with 30 μL molecular grade water. The final concentration of the combined digested DNA was 350 ng/μL (measured by nanodrop). Digestions and purifications performed led to 50% recovery yields of the digested pBR322 DNA.

Cleavage Assay of Linearized pBR322: A fresh 500 μM stock solution of diazonium salt compound 28 was prepared by dissolving 10 μmol of the salt in 20 mL of ddH₂O. In 1.5 mL Eppendorf tubes, a serial dilution was performed to achieve a concentration range between 500 nM and 2.5 nM solutions of the diazonium salt in ddH₂O. In 0.2 mL PCR tubes, 0.5 μg of EcoRV-HF digested pBR322 (from the purified sample) and 20 μL of the corresponding diazonium solutions were mixed. The reaction mixtures were incubated at 25° C. in between two Kessil pro160 green LED (525 nm) lights (44 W) for 1.5 hours. The cleavage assay of compound 28 on linear pBR322 was performed a second time, with the addition of 0.5 μg of supercoiled pBR322 (1 μg/μL) as an internal standard, after the addition of the 6× purple loading dye and immediately before loading the samples in the gel. The DNA-cleaving properties of the diazonium salt compound 28 were then examined by the standard agarose gel electrophoresis procedure to obtain the does-response plot and an EC₅₀ value.

### Cell Culture and Cell Viability Assay:

General cell culture protocol: HeLa cells were grown in DMEM growth media (GIBCO 11995065) with 10% FBS (GIBCO A5670401) in 75 cm² surface treated culture dishes (Fisherbrand FB012937) and were incubated in a cell culture chamber (37° C. with 5% CO₂). Cells were kept at a maximum confluency of 80% and passaged up to 20 times. For each passage, the cells were washed with 1×PBS (GIBCO 20012027), detached from the plate by incubation with 0.25% Trypsin-EDTA (GIBCO 25200072) for 3-4 minutes in the cell culture chamber, and then diluted with growth media. The cells were spun down at 300×g at 4° C. for 5 minutes, resuspended with fresh growth media, and replated in a new flask.

Cell viability assay and IC₅₀ procedure: The media in the cell culture dish was aspirated, and the cells were washed with 1×PBS. The cells were detached from the plate by 4-minute incubation with 0.25% Trypsin-EDTA in the cell culture chamber. The cells were then diluted with DMEM and were spun down at 300×g at 4° C. for 5 minutes. The media was aspirated, and the cell pellet was resuspended in fresh DMEM. The cells were counted and seeded in 6-well culture plates at 50,000 cells/well and incubated overnight. Alongside the 6-well plates, 35 mm glass bottom confocal plates (Fisher Scientific NC0409658) were also seeded with 50,000 cells/plate. 24 hours after the initial seeding, the media in the plates were aspirated and the cells were washed with 1×PBS. A 100 μM stock solution of diazonium compound 28 was prepared in 1×PBS pH 6.6 and a serial dilution was performed to achieve a concentration range of 20 μM to 1.25 μM of compound 28 in 1×PBS pH 6.6. The wash buffer was aspirated from the plates and 2 mL of the corresponding diazonium dilutions was added to the respective wells in the 6-well plates, all in triplicate, in addition to the confocal plates. All plates were incubated in ambient light for 45 minutes in the cell culture hood and then for 1 hour in the cell culture chamber (37° C. with 5% CO₂). Control wells with 2 mL 1×PBS pH 6.6 alone with no diazonium as well as additional control wells with 2 mL DMEM alone with no diazonium, all in triplicate, were also incubated alongside the wells with the diazonium solutions. The plates were then removed from the incubators and the solutions from all the dishes were aspirated and replaced with 2 mL of fresh DMEM. The plates were incubated in the cell culture chamber for 3 days. After the 3-day incubation, the respective cell viability of each well was measured using the PrestoBlue™ Cell Viability Procedure. To each well was added 200 μL of 10× PrestoBlue™ Cell Viability Reagent (ThermoFisher Scientific A13261) and the plates were then incubated in the cell culture chamber for 3 hours. The plates were then analyzed using a plate reader (SpectraMax iD5) by measuring the fluorescence at an excitation of 560 nm and emission of 590 nm. The data was exported from SoftMax Pro7.1 software and used to calculate the relative viability of each well compared with the control wells. The values were plotted against the corresponding diazonium concentrations in a dose-response plot to generate the IC₅₀ value. The cells in the 35 mm glass bottom confocal plates were also imaged after the 3-day incubation using confocal microscopy. Brightfield imaging was performed using a Leica SP8 confocal microscope equipped with a 10× objective. The TLD PMT Trans detector was used to capture transmitted light images of the cell culture plates.

DNA-cleavage assay with deoxygenated water: A sample of double distilled water (ddH₂O) was freeze-pump-thawed (degassed) four times and then brought into an argon-filled glovebox. The subsequent serial dilution and reaction setup were performed in an argon-filled-glovebox. Similar to the standard assay, a fresh 500 μM stock solution of diazonium salt compound 28 was prepared by dissolving 2.5 μmol (1.6 mg) of the salt in 4 mL of the deoxygenated ddH₂O. In 1.5 mL Eppendorf tubes, a serial dilution was performed to achieve a range between 500 nM and 1 nM solutions of the diazonium salt in the deoxygenated ddH₂O. In 0.2 mL PCR tubes, 0.5 μL (0.5 μg) of aqueous supercoiled pBR322 plasmid DNA (1 μg/μL stock) and 20 μL of the corresponding diazonium solutions were mixed. The reaction mixtures were taken out of the glovebox and incubated at 25° C. in between two Kessil pro160 green LED (525 nm) lights (44 W) for 1.5 hours. The DNA-cleaving properties of the diazonium salt under deoxygenated conditions were then examined by the standard agarose gel electrophoresis procedure. A standard reaction with normal ddH2O was setup outside the glovebox and performed alongside as a control. It was also examined by the standard agarose gel electrophoresis procedure. The dose-response plots were generated for each to obtain their respective EC₅₀ values.

UV-Vis experiments: 15.4 μg/mL (25 μM/bp) solutions of supercoiled pBR322 in water and linearized (EcoRV-HF-digested) pBR322 in water were prepared and their absorbances were measured. A 10 μM solution of compound 28 in water was prepared and its absorbance was immediately measured. A solution containing both compound 28 (10 PM) and pBR322 (at 25 μM/bp) was prepared and immediately measured as well as was measured after being left in the dark for 20 minutes. Linearized pBR322 DNA (25 μM/bp) mixed with compound 28 (10 μM) was prepared and measured. Compound 28 (10 μM) and supercoiled pBR322 (25 μM/bp) was prepared and absorbance was measured after irradiation with 525 nm light for 20 minutes.

### Example 1. Synthesis of Diazonium Compounds

A library of diazonium salts were synthesized from various aryl amine precursors under modified diazotization conditions (Starkey, 1939), offering a wide range of functionalities and structural motifs. The general procedure for the coupling and reduction of the aryl amine precursors is as follows:

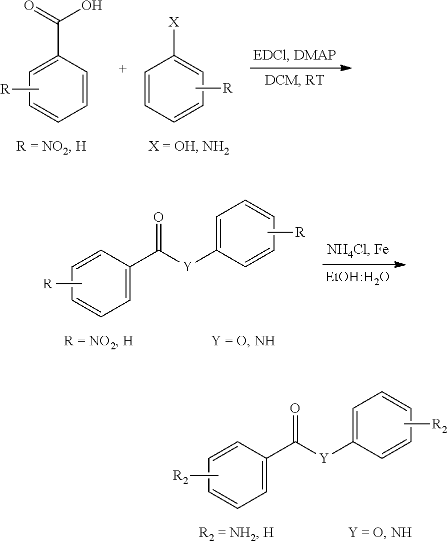

In a round bottom flask, EDCI (1-ethyl-3-(3-dimethylaminopropyl)-carbodiimide hydrochloride) (1.2 equiv.) and DMAP (4-dimethylaminopyridine) (0.1 equiv.) were added to a mixture of a corresponding carboxylic acid in DCM (0.2 M) at 0° C. The mixture was removed from the ice-water bath and allowed to stir for 30 minutes. The subsequent aryl amine or alcohol was then added to the flask, which was then purged with nitrogen and allowed to stir for 16 hours. The precipitate was collected by filtration, washed with DCM, and dried over vacuum to yield the diaryl amide or ester product, which was used in the next step without further purification.

In a round bottom flask, the corresponding nitroarene was dissolved in ethanol (0.25 M). A prepared aqueous solution of ammonium chloride (10 equiv.) in the same volume as ethanol used, was added to the flask, followed by Fe(0) powder (10 equiv.). The flask was sealed with a condenser and refluxed in an oil bath at 100° C. for 3 hours. The mixture was allowed to cool to room temperature and was filtered through a layer of silica. The filtrate was concentrated by solvent evaporation. The resulting solid was washed with water and the solid was collected through vacuum filtration and washed with additional water to obtain the amine product without further purification. When further purification was required, the substrate was purified by flash column chromatography (hexanes:ethyl acetate 1:1).

Some of the synthesized diazonium precursors include:

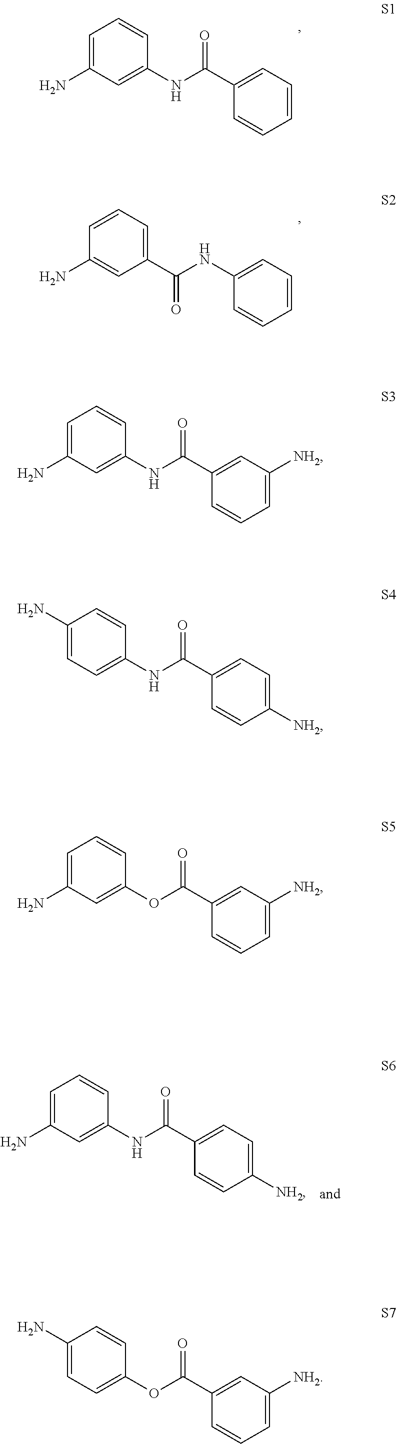

The generic structure of the aryl diazonium compounds studied herein is represented as follows:

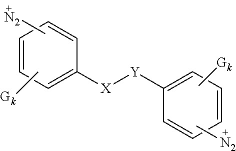

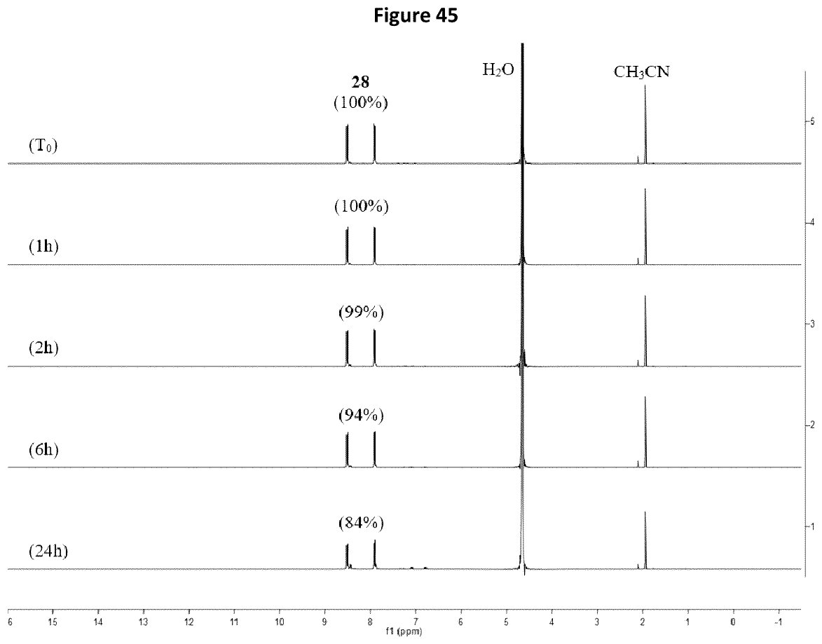

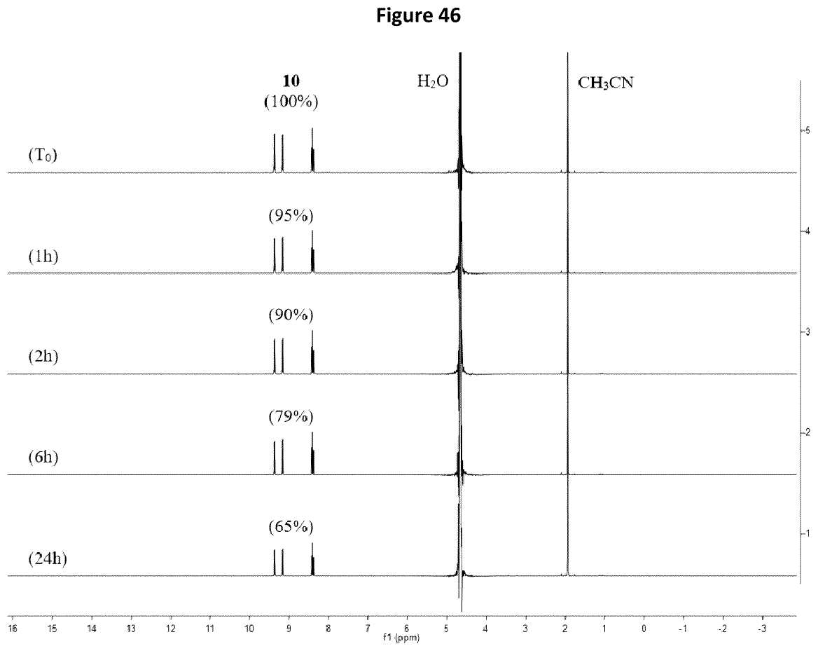

Thirty distinct aryl diazonium compounds were synthesized, containing mono-, bis-, tri-, and tetra-diazonium salts, through the conversion of aryl amines using tert-butyl nitrite. Diethyl ether or acetone as a cosolvent to water facilitated the precipitation of diazonium salts, which were obtained in yields ranging from 40-95%. After washing with ether, the diazonium compounds were confirmed to be pure by ¹H and ¹³C NMR spectroscopy and were used without further purification. The synthesis of aryl diazonium salts was highly efficient, rapid, and required minimal steps using commercially available materials. The route to synthesize these unique diazonium salts involved as few as three steps or even a single 30-minute reaction. Most of these diazonium compounds proved stable in solid form for weeks and in aqueous solutions for longer than 6 hours. The stability of diazonium compounds 28 and 10 was tested in water. 8 mg of compound 28 and 4 mg of compound 10 (˜10 mmol) were dissolved in 1 mL of deuterated water in two separate vials. 0.5 μL of acetonitrile was added as an internal standard to each vial. The solutions were added to NMR tubes and the NMR spectra were taken at 0-, 1-, 2-, 6-, and 24-hour time points (FIG. 45 and FIG. 46). The mol ratio of the diazonium to internal standard was calculated for each spectrum (diazonium/IS) and the ratio from each time point was compared to the ratio from the starting spectra to calculate the percentage of diazonium in solution at each point.

For synthesis of monodiazonium salts, General Procedure 1 was performed as follows:

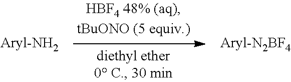

In a 20 mL glass vial, 1 mmol of corresponding amine was suspended in a mixture of 2 mL diethyl ether and 0.6 mL of a 48% aqueous solution of HBF₄. The vial was placed in an ice-brine bath and tert-butyl nitrite (5 equivalents) was added dropwise to the mixture after which precipitate formed in the mixture. The slurry was allowed to stir in the ice-brine bath for 30 minutes. The solid was filtered off and washed with diethyl ether (2×10 mL). The collected diazonium salt was dried over vacuum for 5 minutes and collected without further purification.

Benzenediazonium 1 was prepared according to General Procedure 1 with aniline (93 mg, 91 μL, 1.00 mmol). Benzenediazonium 1 was obtained as a white solid (181 mg, 94%). The NMR matched previously reported spectra (Pfaff, et al., 2021): ¹H NMR (400 MHz, CD₃CN) δ 8.46 (ddd, J=8.8, 2.8, 1.3 Hz, 2H), 8.27 (ddt, J=8.0, 7.4, 1.3 Hz, 1H), 8.03-7.88 (m, 2H).

4-nitro-1-benzene-diazonium 2 was prepared according to General Procedure 1 with 4-nitroaniline (138 mg, 1.00 mmol). 4-nitro-1-benzene-diazonium 2 was obtained as a white solid (193 mg, 0.815 mmol, 82%). The NMR matched previously reported spectra (Pfaff, et al., 2021): ¹H NMR (400 MHz, CD₃CN) δ 8.76-8.70 (m, 2H), 8.65-8.59 (m, 2H).

4-nitrile-benzene-1-diazonium 3 was prepared according to General Procedure 1 with 4-aminobenzonitrile (118 mg, 1.00 mmol). 4-nitrile-benzene-1-diazonium 3 was obtained as a white solid (178 mg, 82%). The NMR matched previously reported spectra (Bremerich, et al., 2019): ¹H NMR (400 MHz, CD₃CN) δ 8.61 (dt, J=9.0, 1.2 Hz, 2H), 8.27-8.22 (m, 2H).

Methyl 4-benzoate-1-diazonium 4 was prepared according to General Procedure 1 with methyl 4-aminobenzoate (151 mg, 1.00 mmol). Methyl 4-benzoate-1-diazonium 4 was obtained as a white solid (200 mg, 0.800 mmol, 80%). The NMR matched previously reported spectra (Heinrich, et al., 2007): ¹H NMR (400 MHz, CD₃CN) δ 8.60 (dq, J=8.9, 1.9 Hz, 2H), 8.46-8.37 (m, 2H), 3.98 (s, 3H); ¹³C NMR (101 MHz, CD₃CN) δ 164.63, 142.13, 133.73, 132.81, 119.35, 54.16; HRMS: m/z (ESI) Calcd for C₈H₇N₂O₂[M]⁺: 163.0502, found: 163.0492.

4-methoxy-benzene-1-diazonium 5 was prepared according to General Procedure 1 with 4-methoxyaniline (123 mg, 1.00 mmol). 4-methoxy-benzene-1-diazonium 5 was obtained as a white solid (186 mg, 84%). The NMR matched previously reported spectra (Pfaff, et al., 2021): ¹H NMR (400 MHz, CD₃CN) δ 8.42-8.36 (m, 2H), 7.42-7.28 (m, 2H), 4.06 (s, 3H).

Naphthalene-1-diazonium 6 was prepared according to General Procedure 1 with 1-naphthylamine (143 mg, 1.00 mmol). Naphthalene-1-diazonium 6 was obtained as a purple solid (168 mg, 0.694 mmol, 69%). The NMR matched previously reported spectra (Tabey, et al., 2018): ¹H NMR (400 MHz, CD₃CN) δ 8.93 (dt, J=7.9, 1.2 Hz, 1H), 8.89-8.84 (m, 1H), 8.37 (d, J=8.3 Hz, 1H), 8.26 (dt, J=8.4, 0.9 Hz, 1H), 8.11 (ddd, J=8.4, 7.0, 1.2 Hz, 1H), 8.01-7.93 (m, 2H); HRMS: m/z (ESI) Calcd for C₁₀H₇N₂[M]⁺: 155.0604, found: 155.0573.

4-nitro-naphthalene-1-diazonium 7 was prepared according to General Procedure 1 with 4-nitro-1-naphthylamine (188 mg, 1.00 mmol). 4-nitro-naphthalene-1-diazonium 7 was obtained as a yellow solid (228 mg, 0.794 mmol, 79%). The characterization data was as follows: ¹H NMR (400 MHz, CD₃CN) δ 9.10 (d, J=8.5 Hz, 1H), 8.48 (dt, J=8.7, 0.9 Hz, 1H), 8.45-8.37 (m, 2H), 8.24 (ddd, J=8.4, 7.1, 1.1 Hz, 1H), 8.15 (ddd, J=8.4, 7.1, 1.1 Hz, 1H); ¹³C NMR (126 MHz, CD₃CN) δ 155.71, 138.05, 134.84, 133.82, 130.33, 125.91, 125.59, 123.27, 123.08, 115.52; ¹⁹F NMR (471 MHz, CD₃CN) δ −151.54; HRMS: m/z (ESI) Calcd for C₁₀H₆N₃O₂ [M]⁺: 200.0455, found: 200.0386.

N-(3-diazobenzene) benzamide 8 was prepared according to General Procedure 1 with N-(3-aminobenzene) benzamide (212 mg, 1.00 mmol). N-(3-diazobenzene) benzamide 8 was obtained as a pale orange solid (274 mg, 87%). The characterization data was as follows: ¹H NMR (400 MHz, CD₃CN) δ 9.44 (s, 1H), 9.36 (t, J=2.2 Hz, 1H), 8.22 (dddt, J=9.2, 8.3, 1.8, 0.9 Hz, 2H), 8.04-7.94 (m, 2H), 7.89 (t, J=8.4 Hz, 1H), 7.70-7.62 (m, 1H), 7.57 (ddd, J=8.2, 6.5, 1.3 Hz, 2H); ¹³C NMR (126 MHz, CD₃CN) δ 167.58, 142.48, 134.38, 133.66, 133.50, 133.09, 129.70, 128.68, 128.38, 121.70, 115.84; ¹⁹F NMR (471 MHz, CD₃CN) δ −151.61.

N-phenyl 3-diazobenzoamide 9 was prepared according to General Procedure 1 with N-phenyl 3-aminobenzamide (212 mg, 1.00 mmol). N-phenyl 3-diazobenzoamide 9 was obtained as a pale-yellow solid (210 mg, 68%). The characterization data was as follows: ¹H NMR (400 MHz, CD₃CN) δ 9.10 (s, 1H), 8.98 (t, J=1.9 Hz, 1H), 8.70 (ddd, J=7.9, 1.7, 1.0 Hz, 1H), 8.61 (ddd, J=8.4, 2.2, 1.0 Hz, 1H), 8.08 (t, J=8.2 Hz, 1H), 7.76-7.68 (m, 2H), 7.43 (dd, J=8.5, 7.4 Hz, 2H), 7.30-7.18 (m, 1H); ¹³C NMR (101 MHz, CD₃CN) δ 162.27, 140.82, 139.37, 138.84, 135.20, 133.06, 132.76, 129.91, 125.96, 121.61, 116.62.

For synthesis of multidiazonium salts, General Procedure 2 was performed as follows:

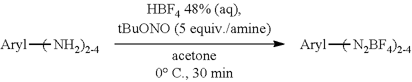

In a 20 mL glass vial, 0.200-1.00 mmol of corresponding amine was suspended in a mixture of 2 mL acetone and 1 mL of a 48% aqueous solution of HBF₄. The vial was placed in an ice-brine bath and tert-butyl nitrite (5 equivalents per amine) was added dropwise to the mixture after which precipitate formed in the mixture. The slurry was allowed to stir in the ice-brine bath for 30 minutes. The solid was filtered off and washed with diethyl ether (2×10 mL). The collected diazonium salt was dried over vacuum for 5 minutes and collected without further purification.

Naphthalene-1,5-bisdiazonium 10 was prepared according to General Procedure 2 with naphthalene-1,5-diamine (158 mg, 1.00 mmol). Naphthalene-1,5-bisdiazonium 10 was obtained as a beige solid (290 mg, 82%). The characterization data was as follows: ¹H NMR (400 MHz, CD₃CN) δ 9.30 (dd, J=7.9, 0.9 Hz, 2H), 9.09 (d, J=8.6 Hz, 2H), 8.46 (t, J=8.3 Hz, 2H); ¹³C NMR (126 MHz, CD₃CN) δ 141.90, 137.31, 132.93, 128.75; ¹⁹F NMR (471 MHz, CD₃CN) δ −146.20; HRMS: m/z (ESI) Calcd for C₁₀H₆N₄BF₄ [M]⁺: 269.0616, found: 269.0611; IR (cm⁻¹): 3101, 2284, 1505, 1353, 1248, 1203, 1026, 829, 806.

Benzene-1,4-bisdiazonium 11 was prepared according to General Procedure 2 with benzene-1,4-diamine (108 mg, 1.00 mmol). Benzene-1,4-bisdiazonium 11 was obtained as a dark yellow solid (202 mg, 67%). The characterization data was as follows: ¹H NMR (400 MHz, CD₃CN) δ 8.97 (s, 4H); ¹⁹F NMR (471 MHz, CD₃CN) δ −151.40; IR (cm⁻¹): 3105, 2320, 1303, 1028, 860.

N-(3-diazobenzene) 3-diazobenzamide 12 was prepared according to General Procedure 2 with N-(3-aminobenzene) 3-aminobenzamide (227 mg, 1.00 mmol). N-(3-diazobenzene) 3-diazobenzamide 12 was obtained as a pale orange solid (330 mg, 77%). The characterization data was as follows: ¹H NMR (500 MHz, CD₃CN) δ 9.81 (s, 1H), 9.25 (t, J=2.2 Hz, 1H), 9.02 (t, J=2.0 Hz, 1H), 8.73 (dt, J=8.1, 1.4 Hz, 1H), 8.68 (ddd, J=8.4, 2.2, 1.0 Hz, 1H), 8.38-8.25 (m, 2H), 8.12 (t, J=8.2 Hz, 1H), 7.95 (t, J=8.4 Hz, 1H); ¹³C NMR (126 MHz, CD₃CN) δ 163.32, 141.43, 141.10, 137.79, 136.05, 133.78, 133.40, 133.35, 133.02, 129.39, 122.25, 117.02, 116.21; ¹⁹F NMR (471 MHz, CD₃CN) δ −151.03; HRMS: m/z (ESI) Calcd for C₁₃H₉N₅O [M]⁺: 251.0796, found: 251.0767.

N-(3-diazobenzene) 4-diazobenzamide 13 was prepared according to General Procedure 2 with N-(3-aminobenzene) 4-aminobenzamide (90 mg, 0.396 mmol). N-(3-diazobenzene) 4-diazobenzamide 13 was obtained as a yellow solid (154 mg, 92%). The characterization data was as follows: ¹H NMR (500 MHz, CD₃CN) δ 9.80 (s, 1H), 9.25 (t, J=2.2 Hz, 1H), 8.68-8.62 (m, 2H), 8.38-8.35 (m, 2H), 8.31-8.24 (m, 2H), 7.94 (t, J=8.4 Hz, 1H); 13C NMR (126 MHz, CD₃CN) δ 164.53, 145.76, 141.40, 133.85, 133.81, 133.42, 131.75, 129.47, 122.24, 116.22; ¹⁹F NMR (471 MHz, CD₃CN) δ −151.44; HRMS: m/z (ESI) Calcd for C₁₃H₁₀N₅OBF₄ [M+H]⁺: 339.0904, found: 338.0728; IR (cm⁻¹): 3351, 2303, 2271, 1704, 1605, 1574, 1532, 1483, 1417, 1335, 1310, 1262, 1037, 1003, 994, 950, 915, 888, 853, 833, 818, 768, 739, 673.

N-(4-diazobenzene) 3-diazobenzamide 14 was prepared according to General Procedure 2 with N-(4-aminobenzene) 3-aminobenzamide (80.0 mg, 0.352 mmol). N-(4-diazobenzene) 3-diazobenzamide 14 was obtained as a yellow solid (116 mg, 78%). The characterization data was as follows: ¹H NMR (400 MHz, CD₃CN) δ 10.05 (s, 1H), 9.00 (t, J=1.9 Hz, 1H), 8.71 (dddd, J=13.3, 8.4, 1.9, 1.0 Hz, 2H), 8.54-8.43 (m, 2H), 8.34-8.22 (m, 2H), 8.13 (t, J=8.2 Hz, 1H); ¹³C NMR (126 MHz, CD₃CN) δ 163.69, 150.96, 141.31, 137.69, 136.26, 135.60, 133.39, 133.13, 122.59, 117.08, 106.54; ¹⁹F NMR (471 MHz, CD₃CN) δ −151.25; HRMS: m/z (ESI) Calcd for C₁₃H₉N₅OBF₄ [M]⁺: 338.0825, found: 338.0789.

N-(4-diazobenzene) 4-diazobenzamide 15 was prepared according to General Procedure 2 with N-(4-aminobenzene) 4-aminobenzamide (227 mg, 1.00 mmol). N-(4-diazobenzene) 4-diazobenzamide 15 was obtained as a yellow solid (380 mg, 89%). The characterization data was as follows: ¹H NMR (400 MHz, CD₃CN) δ 9.98 (s, 1H), 8.74-8.58 (m, 2H), 8.52-8.42 (m, 2H), 8.41-8.31 (m, 2H), 8.29-8.20 (m, 2H); ¹³C NMR (126 MHz, CD₃CN) δ 164.92, 150.89, 145.54, 135.62, 133.86, 131.89, 122.61, 118.95, 106.67; ¹⁹F NMR (471 MHz, CD₃CN) δ −151.36; HRMS: m/z (ESI) Calcd for C₁₃H₁₀N₅O [M+H]⁺: 252.0874, found: 252.0864.

(3-diazophenyl) 3-diazobenzoate 16 was prepared according to General Procedure 2 with (3-aminophenyl) 3-aminobenzoate (227 mg, 1.00 mmol). (3-diazophenyl) 3-diazobenzoate 16 was obtained as a beige solid (250 mg, 59%). The characterization data was as follows: ¹H NMR (400 MHz, CD₃CN) δ 9.25 (t, J=2.2 Hz, 1H), 8.93 (dt, J=8.2, 1.4 Hz, 1H), 8.79 (ddd, J=8.4, 2.2, 1.1 Hz, 1H), 8.56 (t, J=2.2 Hz, 1H), 8.51 (ddd, J=8.2, 2.2, 1.1 Hz, 1H), 8.23 (ddd, J=8.5, 2.2, 1.1 Hz, 1H), 8.17 (t, J=8.2 Hz, 1H), 8.07 (t, J=8.4 Hz, 1H); ¹³C NMR (126 MHz, CD₃CN) δ 161.35, 151.64, 143.29, 137.70, 137.04, 134.73, 134.39, 133.77, 132.87, 131.88, 117.47, 116.62; ¹⁹F NMR (471 MHz, CD₃CN) δ −151.26; HRMS: m/z (ESI) Calcd for C₁₃H₉N₄O₂BF₄ [M+H]⁺: 339.0665, found: 339.0607.

Benzophenone-4,4′-bisdiazonium 17 was prepared according to General Procedure 2 with 4,4′-diaminobenzophenone (212 mg, 1.00 mmol). Benzophenone-4,4′-bisdiazonium 17 was obtained as a pale-yellow solid (400 mg, 98%). The characterization data was as follows: ¹H NMR (400 MHz, CD₃CN) δ 8.68-8.65 (m, 4H), 8.22-8.19 (m, 4H); ¹³C NMR (101 MHz, CD₃CN) δ 191.84, 146.06, 133.88, 133.26, 119.72; ¹⁹F NMR (471 MHz, CD₃CN) δ −151.36; HRMS: m/z (ESI) Calcd for C₁₃H₉N₄OBF₄ [M+H]⁺: 323.0716, found: 323.0642.

Benzophenone-3,3′-bisdiazonium 18 was prepared according to General Procedure 2 with 3,3′-diaminobenzophenone (80.0 mg, 0.377 mmol). Benzophenone-3,3′-bisdiazonium 18 was obtained as a pale-yellow solid (109 mg, 71%). The characterization data was as follows: ¹H NMR (400 MHz, CD₃CN) δ 8.85 (t, J=1.9 Hz, 2H), 8.77 (ddd, J=8.4, 2.1, 1.1 Hz, 2H), 8.61 (ddd, J=8.0, 1.7, 1.1 Hz, 2H), 8.15 (t, J=8.2 Hz, 2H); ¹³C NMR (101 MHz, CD₃CN) δ 188.60, 143.12, 138.72, 136.94, 134.50, 133.61, 117.11; ¹⁹F NMR (471 MHz, CD₃CN) δ −151.25; HRMS: m/z (ESI) Calcd for C₁₃H₈N₄OBF₄ [M+BF₄]⁺: 323.0716, found: 323.0693.

Di(benzene-4-diazonium)ether 19 was prepared according to General Procedure 2 with 4,4′-oxydianiline (200 mg, 1.00 mmol). Di(benzene-4-diazonium)ether 19 was obtained as a white solid (370 mg, 93%). The characterization data was as follows: ¹H NMR (400 MHz, CD₃CN) δ 8.63-8.55 (m, 1H), 7.65-7.57 (m, 1H); ¹³C NMR (101 MHz, CD₃CN) δ 164.98, 137.09, 123.44, 110.25; ¹⁹F NMR (471 MHz, CD3CN) δ −151.39; HRMS: m/z (ESI) Calcd for C₁₂H₉N₄OBF₄ [M+H]⁺: 311.0716, found: 311.0664.

Di(benzene-3-diazonium)ether 20 was prepared according to General Procedure 2 with 3,3′-oxydianiline (80.0 mg, 0.400 mmol). Di(benzene-3-diazonium)ether 20 was obtained as a pale-yellow solid (129 mg, 81%). The characterization data was as follows: ¹H NMR (400 MHz, CD₃CN) δ 8.40 (dt, J=6.8, 2.1 Hz, 2H), 8.22-8.15 (m, 2H), 8.06-7.96 (m, 4H); ¹³C NMR (101 MHz, CD₃CN) δ 156.85, 134.88, 134.37, 130.22, 122.61, 117.02; ¹⁹F NMR (471 MHz, CD₃CN) δ −151.30; HRMS: m/z (ESI) Calcd for C₁₂H₈N₄OB₂F₈Na [M+Na]⁺: 421.0643, found: 421.0601.

The aryl amine precursor to compound 21 was synthesized during the synthesis of precursor compound S3. Some of the coupled nitro product (1.0 g, 3.5 mmol, 1 equiv.) was methylated with iodomethane (inert conditions, 1.25 equiv. of 60% NaH in 0.2 M THF, stirred at room temperature for 1 hour, then 4 equiv. of iodomethane were added, stirred at room temperature overnight, flash column chromatography 4:1 hexanes:ethyl acetate) and then the methylated nitroarene product was reduced to yield N-(3-aminobenzene)-N-methyl-3-aminobenzamide. N-(3-diazobenzene)-N-methyl-3-diazobenzamide 21 was prepared according to General Procedure 2 with N-(3-aminobenzene)-N-methyl-3-aminobenzamide (241 mg, 1.00 mmol). N-(3-diazobenzene)-N-methyl-3-diazobenzamide 21 was obtained as a beige solid (310 mg, 71%). The characterization data was as follows: ¹H NMR (400 MHz, CD₃CN) δ 8.51-8.46 (m, 2H), 8.41 (t, J=2.2 Hz, 1H), 8.34 (ddd, J=8.4, 2.1, 1.0 Hz, 1H), 8.22-8.15 (m, 1H), 8.14-8.08 (m, 1H), 7.93-7.83 (m, 2H), 3.45 (s, 3H); ¹³C NMR (126 MHz, CD₃CN) δ 166.38, 146.15, 141.85, 141.01, 139.17, 134.57, 133.77, 133.16, 132.64, 131.55, 130.44, 116.55, 38.95; ¹⁹F NMR (471 MHz, CD₃CN) δ −151.22.

N,N′-di(3-benzenediazonium)urea 22 was prepared according to General Procedure 2 with N,N′-di(3-aminobenzene)urea (242 mg, 1.00 mmol). N,N′-di(3-benzenediazonium)urea 22 was obtained as an orange solid (317 mg, 75%). The characterization data was as follows: ¹H NMR (400 MHz, CD₃CN) δ 8.92 (t, J=2.3 Hz, 1H), 8.47 (s, 1H), 8.15 (dd, J=8.2, 2.2 Hz, 1H), 8.06 (dd, J=8.5, 2.3 Hz, 1H), 7.84 (t, J=8.4 Hz, 1H).

(R)-[1,1′-binaphthalene]-2,2′-bisdiazonium 23 was prepared according to General Procedure 2 with (R)-(+)-1,1′-binaphthyl-2,2′-diamine (80.0 mg, 0.281 mmol). (R)-[1,1′-binaphthalene]-2,2′-bisdiazonium 23 was obtained as a yellow solid (93.5 mg, 69%). The characterization data was as follows: ¹H NMR (400 MHz, CD₃CN) δ 8.83 (dd, J=9.2, 0.9 Hz, 2H), 8.66 (d, J=9.2 Hz, 2H), 8.49 (dt, J=8.5, 0.9 Hz, 2H), 8.18 (ddd, J=8.4, 6.9, 1.2 Hz, 2H), 7.90 (ddd, J=8.4, 6.9, 1.2 Hz, 2H), 7.58 (d, J=8.4 Hz, 2H); ¹³C NMR (126 MHz, CD₃CN) δ 140.03, 136.80, 136.76, 135.20, 133.52, 132.22, 131.25, 128.11, 125.28, 114.64; ¹⁹F NMR (471 MHz, CD₃CN) δ −151.50; HRMS: m/z (ESI) Calcd for C₂₀H₁₂N₄BF₄ [M]⁺: 395.1080, found: 395.1069.

(S)-[1,1′-binaphthalene]-2,2′-bisdiazonium 24 was prepared according to General Procedure 2 with (S)-(−)-1,1′-Binaphthyl-2,2′-diamine (80.0 mg, 0.281 mmol). (S)-[1,1′-binaphthalene]-2,2′-bisdiazonium 24 was obtained as a yellow solid (108 mg, 80%). The characterization data was as follows: ¹H NMR (500 MHz, CD₃CN) δ 8.83 (d, J=9.2 Hz, 2H), 8.66 (dd, J=9.2, 1.2 Hz, 2H), 8.49 (d, J=8.4 Hz, 2H), 8.18 (ddd, J=8.4, 6.9, 1.2 Hz, 2H), 7.90 (ddd, J=8.4, 6.9, 1.2 Hz, 2H), 7.58 (d, J=8.4 Hz, 2H); ¹³C NMR (126 MHz, CD₃CN) δ 140.05, 136.82, 135.21, 133.52, 132.26, 131.26, 128.14, 125.25, 114.60; ¹⁹F NMR (471 MHz, CD₃CN) δ −151.28; HRMS: m/z (ESI) Calcd for C₂₀H₁₂N₄BF₄ [M]⁺: 395.1080, found: 395.1058.

Acridine-3,6-bisdiazonium 25 was prepared according to General Procedure 2 with 3,6-diaminoacridine (102 mg, 0.250 mmol). Acridine-3,6-bisdiazonium 25 was obtained as a purple solid (92.3 mg, 91%). The characterization data was as follows: ¹H NMR (400 MHz, CD₃CN) δ 9.70 (dt, J=1.9, 0.8 Hz, 2H), 9.62 (s, 1H), 8.74 (d, J=9.4 Hz, 2H), 8.38 (dd, J=9.3, 2.1 Hz, 2H); ¹³C NMR (101 MHz, CD₃CN) δ 148.32, 142.29, 142.01, 135.27, 133.54, 123.27, 119.94; ¹⁹F NMR (471 MHz, CD₃CN) δ −151.13; IR (cm⁻¹): 3100, 2290, 1601, 1559, 1292, 1036, 1012, 930, 832, 804, 768, 740, 682, 636, 608.

Ethidium bisdiazonium 26 was prepared according to General Procedure 2 with ethidium bromide (98.2 mg, 0.250 mmol). Ethidium bisdiazonium 26 was obtained as a pink solid (127 mg, 85%). The characterization data was as follows: ¹H NMR (400 MHz, CD₃CN) δ 9.76 (d, J=1.9 Hz, 1H), 9.56 (d, J=9.2 Hz, 1H), 9.48 (d, J=9.2 Hz, 1H), 9.15 (ddd, J=17.5, 9.2, 2.1 Hz, 2H), 8.96 (d, J=2.1 Hz, 1H), 8.00 (t, J=7.6 Hz, 1H), 7.92 (t, J=7.6 Hz, 2H), 7.81-7.72 (m, 2H), 4.94 (q, J=7.3 Hz, 2H), 1.65 (t, J=7.3 Hz, 3H); ¹³C NMR (101 MHz, CD₃CN) δ 170.63, 141.21, 140.51, 136.96, 136.23, 134.16, 134.05, 131.99, 131.94, 131.07, 130.40, 129.56, 128.99, 128.77, 128.61, 121.93, 120.59, 54.76, 14.86; ¹⁹F NMR (471 MHz, CD₃CN) δ −151.21; IR (cm⁻¹): 3095, 2310, 1608, 1582, 1447, 1383, 1339, 1298, 1022, 1002, 892, 829, 766, 698, 601.

Tetrakis(benzene-4-diazonium)ethylene 27 was prepared according to General Procedure 2 with 4,4′,4″,4′″-(ethene-1,1,2,2-tetrayl)tetraaniline (118 mg, 0.200 mmol). Tetrakis(benzene-4-diazonium)ethylene 27 was obtained as a yellow solid (145 mg, 92%). The characterization data was as follows: ¹H NMR (400 MHz, CD₃CN) δ 8.42-8.31 (m, 8H), 7.71-7.54 (m, 8H); ¹³C NMR (126 MHz, CD₃CN) δ 151.91, 143.44, 134.91, 133.74, 115.72; ¹⁹F NMR (471 MHz, CD₃CN) δ −151.15; HRMS: m/z (ESI) Calcd for C₂₆H₁₆N₈[M]⁺: 440.1476, found: 440.1555.

Tetrakis(benzene-4-diazonium)methane 28 was prepared according to General Procedure 2 with tetrakis(4-aminobenzene)methane (80.0 mg, 0.210 mmol). Tetrakis(benzene-4-diazonium)methane 28 was obtained as a pale beige solid (140 mg, 86%). The characterization data was as follows: ¹H NMR (400 MHz, CD₃CN) δ 8.52 (d, J=9.1 Hz, 8H), 7.87 (d, J=9.2 Hz, 8H); ¹³C NMR (126 MHz, CD₃CN) δ 154.96, 134.06, 133.97, 115.26, 99.29; ¹⁹F NMR (471 MHz, CD₃CN) δ −151.31; HRMS: m/z (ESI) Calcd for C₂₅H₁₆N₈NaB₄F₁₆ [M+Na+B₄F₁₆]⁺: 799.1523, found: 799.1527; IR (cm⁻¹): 3112, 2284, 1574, 1422, 1291, 1030, 827, 757, 553.

The starting aryl amine to synthesize compound 29 was synthesized according to the general procedure for the coupling and reduction of the aryl amine precursors using N-Boc-1,4-phenylenediamine and benzene-1,3,5-tricarboxylic acid. Instead of an iron-acid reduction, 200 mg (0.25 mmol) of coupled product was treated with 2 mL of TFA and allowed to stir for 30 minutes. The product was crashed out with diethyl ether, filtered by vacuum filtration, and washed with ether to obtain N1,N3,N5-tris(4-aminobenzene)benzene-1,3,5-tricarboxamide (120 mg, quant.). N1,N3,N5-tris(benzene-4-diazonium)benzene-1,3,5-tricarboxamide 29 was prepared according to General Procedure 2 with N1,N3,N5-tris(4-aminobenzene)benzene-1,3,5-tricarboxamide (120 mg, 0.250 mmol). N1,N3,N5-tris(benzene-4-diazonium)benzene-1,3,5-tricarboxamide 29 was obtained as a dark brown solid (140 mg, 72%). The NMR spectra show minor impurity. The compound was used without further purification. The characterization data was as follows: ¹H NMR (400 MHz, CD₃CN) δ 10.02 (s, 3H), 8.82 (s, 3H), 8.52-8.40 (m, 6H), 8.39-8.27 (m, 6H); ¹⁹F NMR (471 MHz, CD₃CN) δ −151.32; ¹³C NMR (101 MHz, CD₃CN) δ 165.43, 151.04, 135.16, 135.04, 131.94, 121.90, 105.09; HRMS: m/z (ESI) Calcd for C₂₇H₁₈N₉O₃B₃F₁₂Na [M+Na]⁺: 800.1501, found: 800.1431.

Methyl 3,5-bisdiazonium-1-benzoate tetrafluoroborate 30 was prepared according to General Procedure 2. The characterization data was as follows: ¹H NMR (400 MHz, CD₃CN) δ 9.76 (t, J=2.0 Hz, 1H), 9.63 (d, J=2.0 Hz, 2H), 4.08 (s, 4H); ¹⁹F NMR (471 MHz, CD₃CN) δ −151.19.

To synthesize diazonium compound 34, 720 mg tetrakis(4-aminobenzene) methane (1.9 mmol, 1 equiv.) was dissolved in 100 mL dry THF in a round bottom flask. Then, 0.6 mL of diisopropylethylamine (2 equiv.) was added and the flask was placed in an ice-brine bath. A 10 mL 0.19 M solution of acetyl chloride in dry THF was prepared (1 equiv.) and added via a syringe pump (1 mL/1 min.) to the flask. The flask was then removed from the bath and allowed to stir at room temperature for 1 hour. TLC analysis confirmed presence of monoacetylated spot. The mixture was concentrated by solvent evaporation and purified by flash column chromatography (0 to 5% MeOH in DCM) to achieve the aryl amine precursor as a white solid (180 mg, 23%). Diazonium compound 34 was prepared according to General Procedure 2 from aryl amine precursor (80.0 mg, 0.210 mmol). Diazonium compound 34 was obtained as a white solid (140 mg, 86%). The characterization data was as follows: ¹H NMR (400 MHz, CD₃CN) δ 8.52 (s, 1H), 8.50-8.43 (m, 6H), 7.93-7.85 (m, 6H), 7.66-7.57 (m, 2H), 7.24-7.12 (m, 2H), 2.06 (s, 3H); ¹³C NMR (101 MHz, CD₃CN) δ 157.86, 134.28, 134.01, 131.72, 120.85, 114.41, 68.40, 24.25.

### Example 2. Structure-Activity Relationship of DNA Cleaving Ability of Diazonium Compounds

With a library of dozens of aryl diazonium salts in hand, the structure-activity-relationship (SAR) for DNA cleavage was studied. To examine the DNA-cleaving ability of diazonium salts, bis-diazonium compound 10, naphthalene-1,5-bisdiazonium tetrafluoroborate, was used as a model compound to develop the protocol for evaluating DNA cleavage potency. A common assay was employed, using supercoiled DNA pBR322 (Form I), which can undergo single- and double-strand cleavage, resulting in nicked (Form II) and linear (Form III) forms, respectively. On agarose gel electrophoresis, the various forms of pBR322 travel different distances, calibrated through cleavage with digestion enzymes such as the nicking enzyme, Nb.BtsI, and the double-strand cleaving enzyme, EcoRV-HF. DNA was incubated with the diazonium salts under green light irradiation (525 nm). Upon photoredox activation without the presence of a photocatalyst, these diazonium compounds induced both double- and single-strand breaks in supercoiled pBR322 plasmid DNA. This led to the conversion of the DNA into its nicked and linear forms, after 1.5-2 hours at 22° C. in neutral pH water. At a low concentration of compound 10, the supercoiled pBR322 underwent single-strand cleavage to afford Form II. As the concentration of compound 10 increased, Form II underwent double-strand cleavage to yield Form III. Further increase in compound 10 concentration led to complete DNA degradation to low molecular weight fragments.

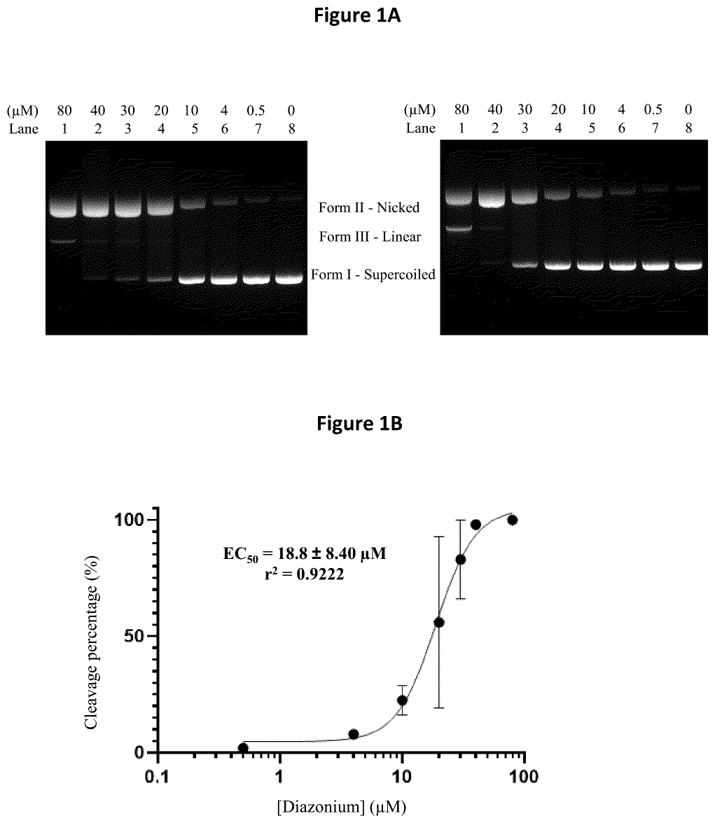

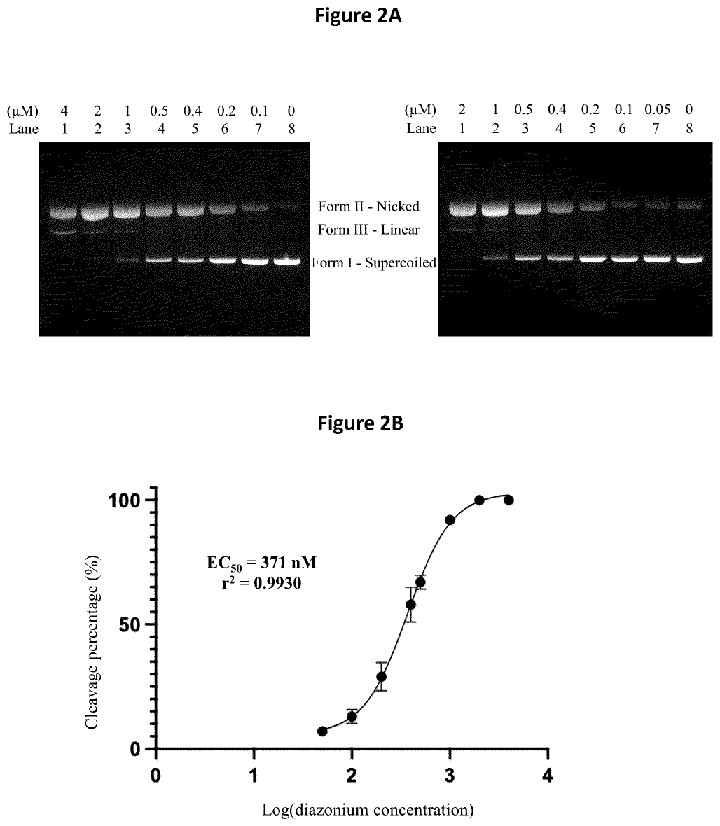

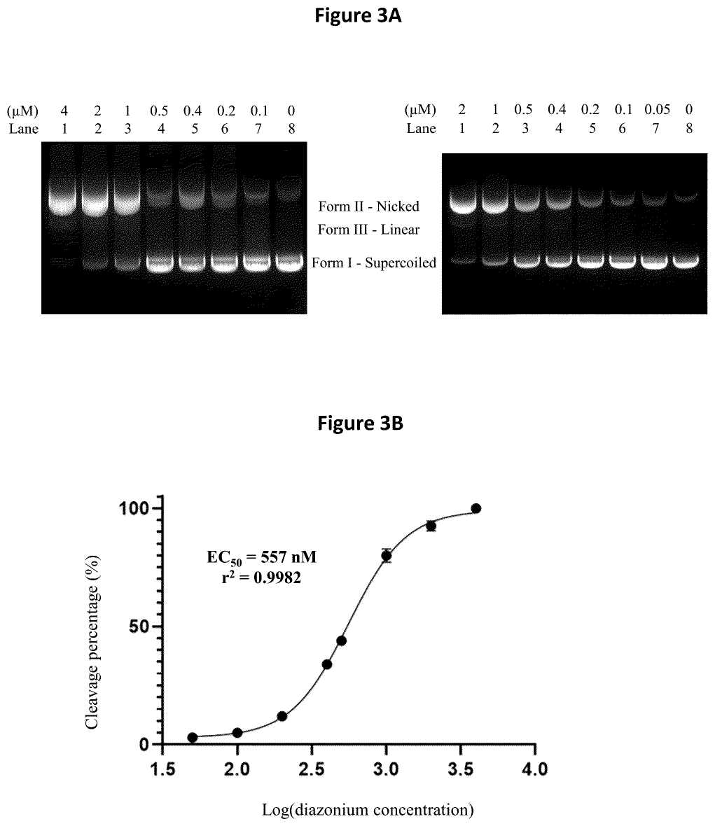

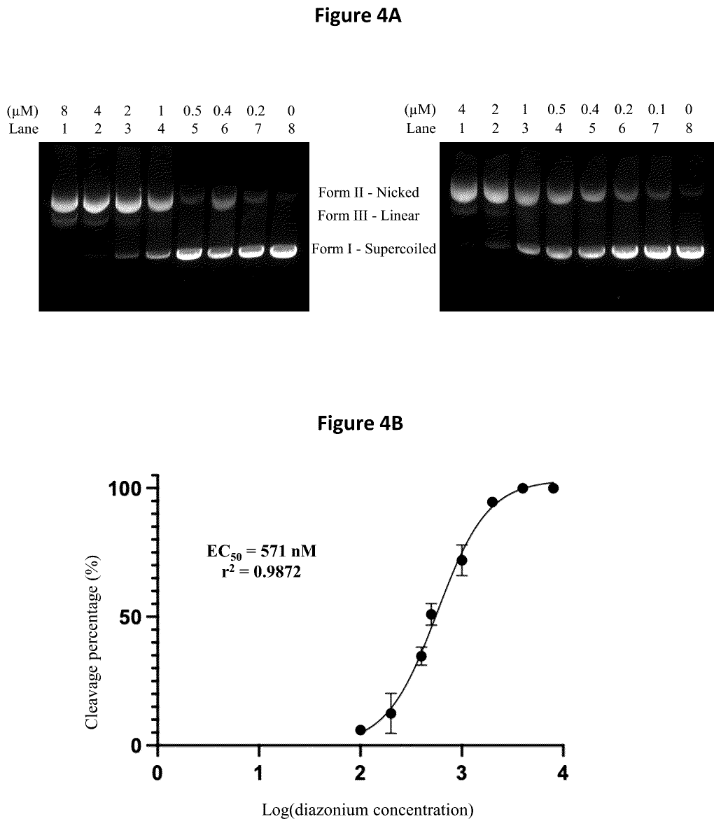

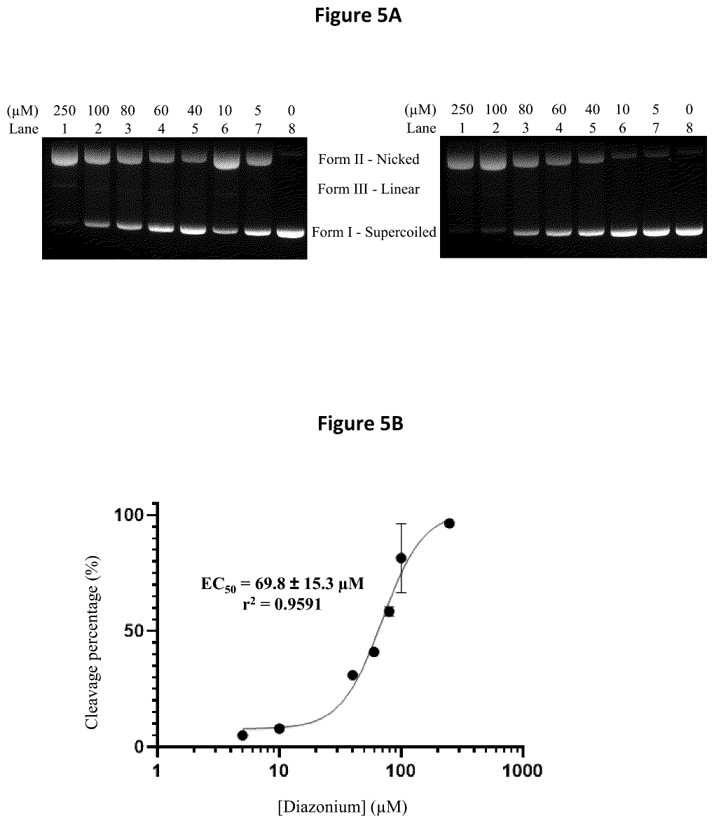

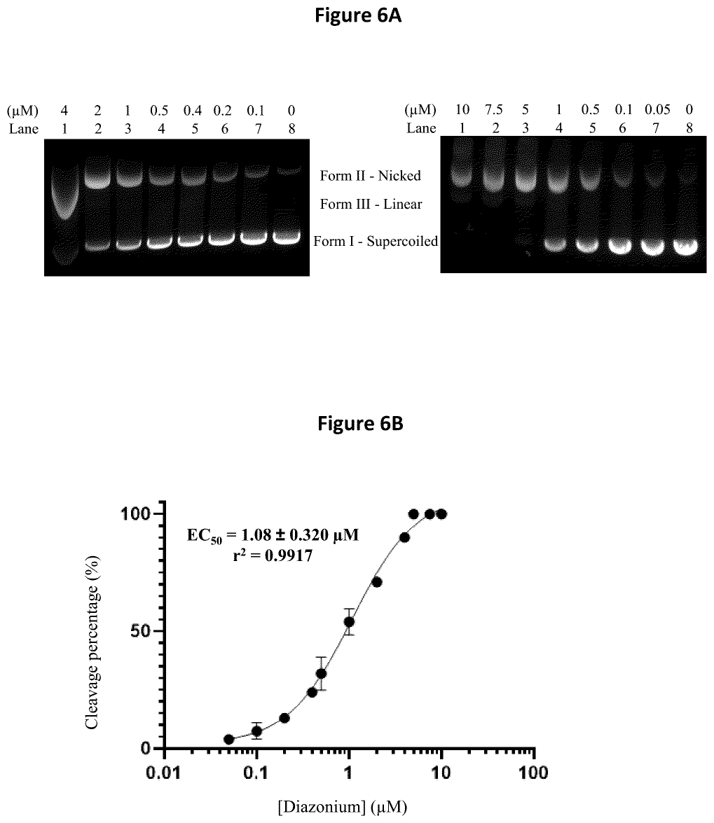

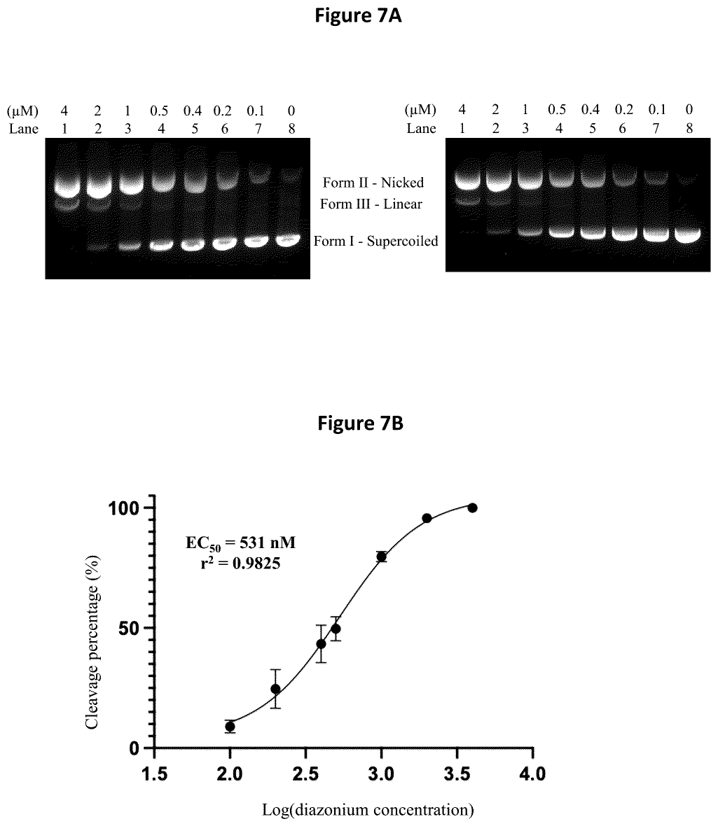

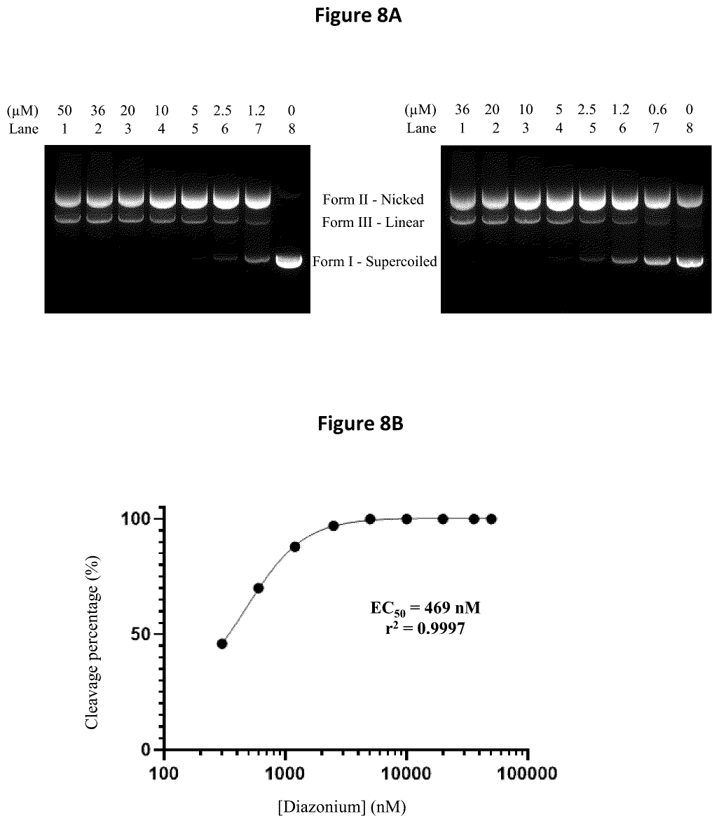

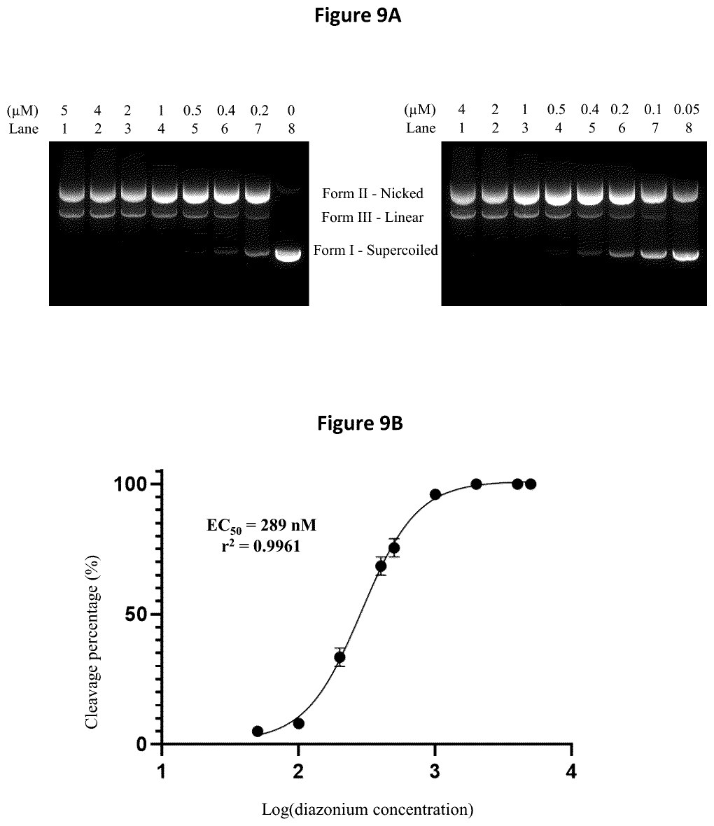

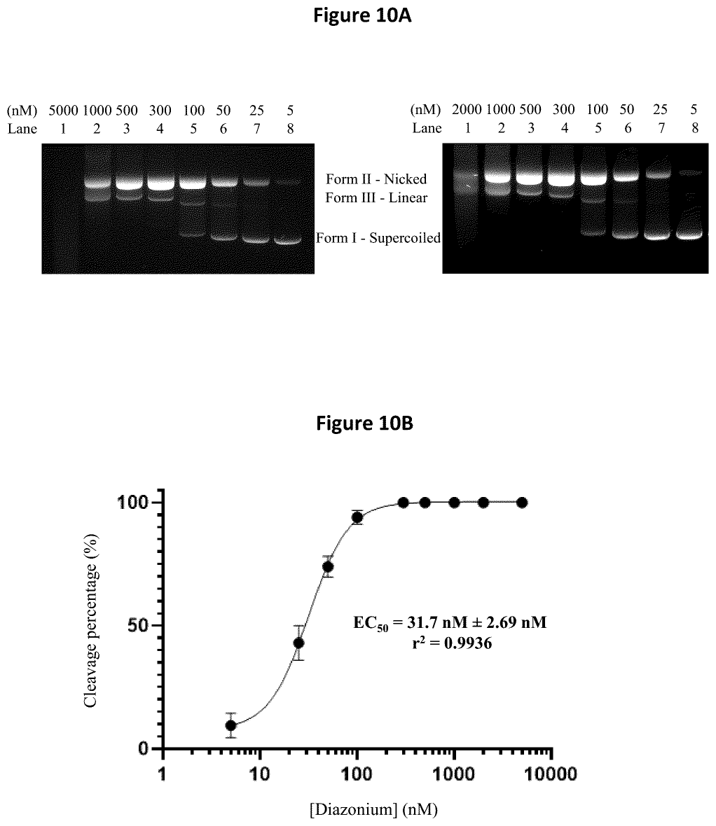

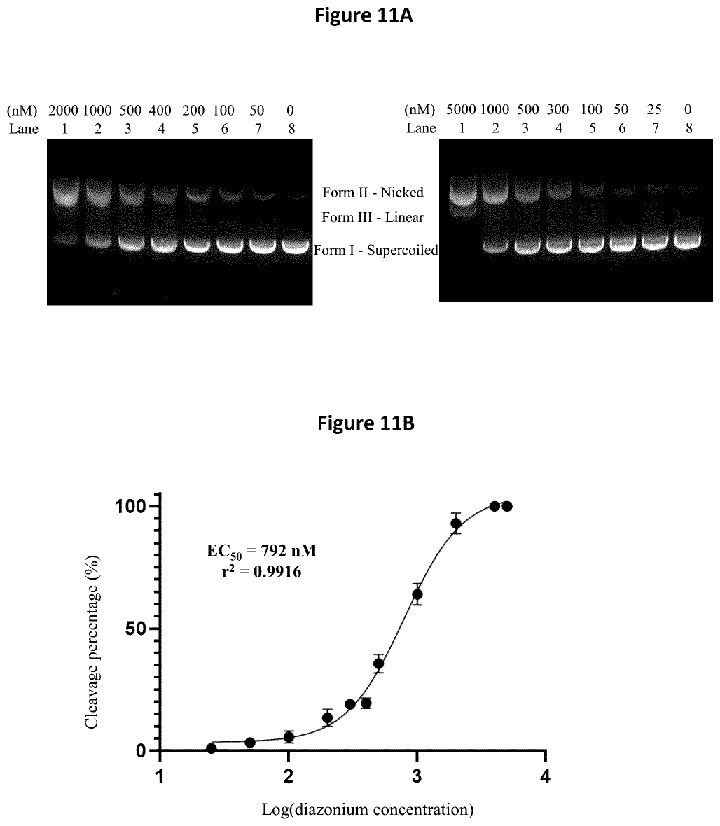

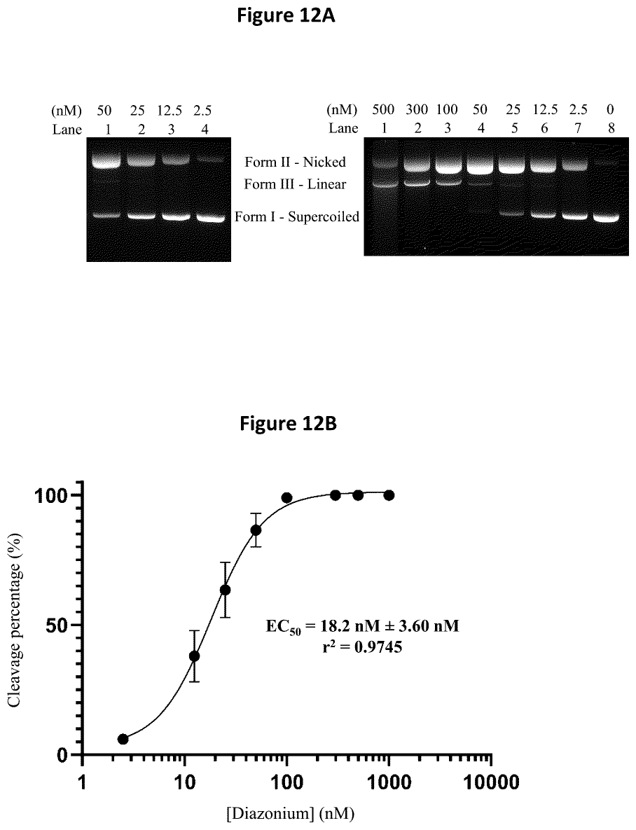

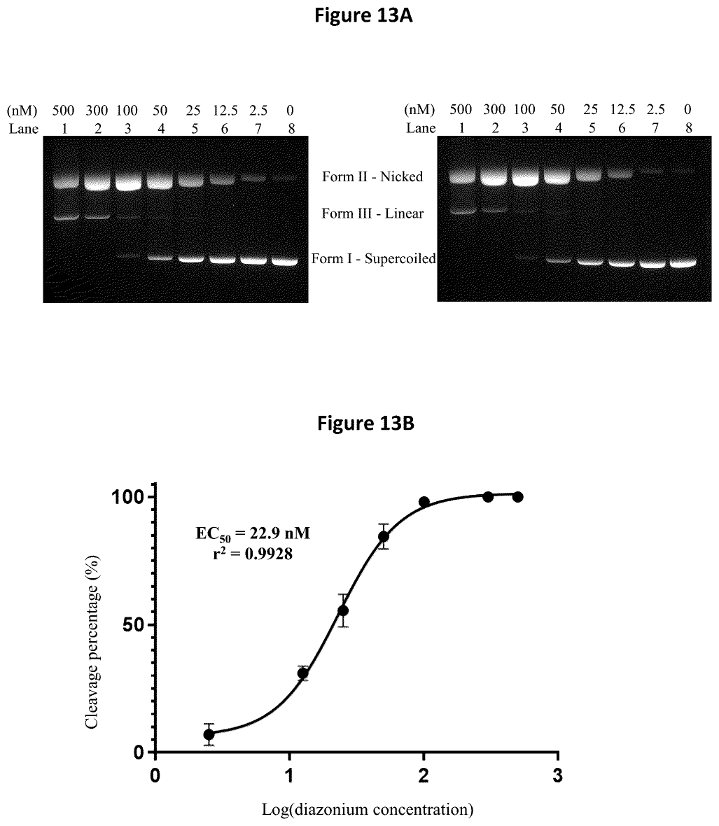

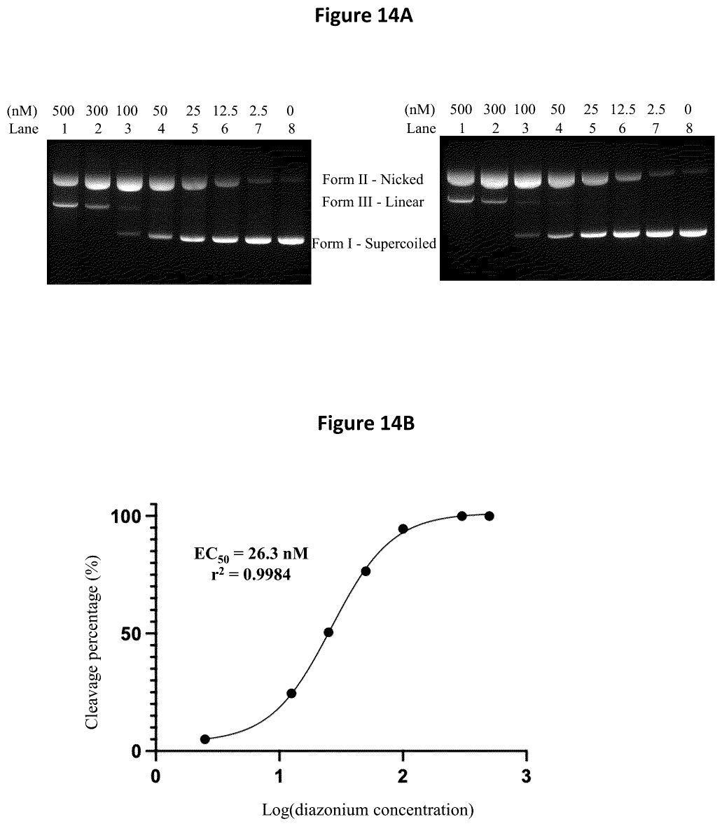

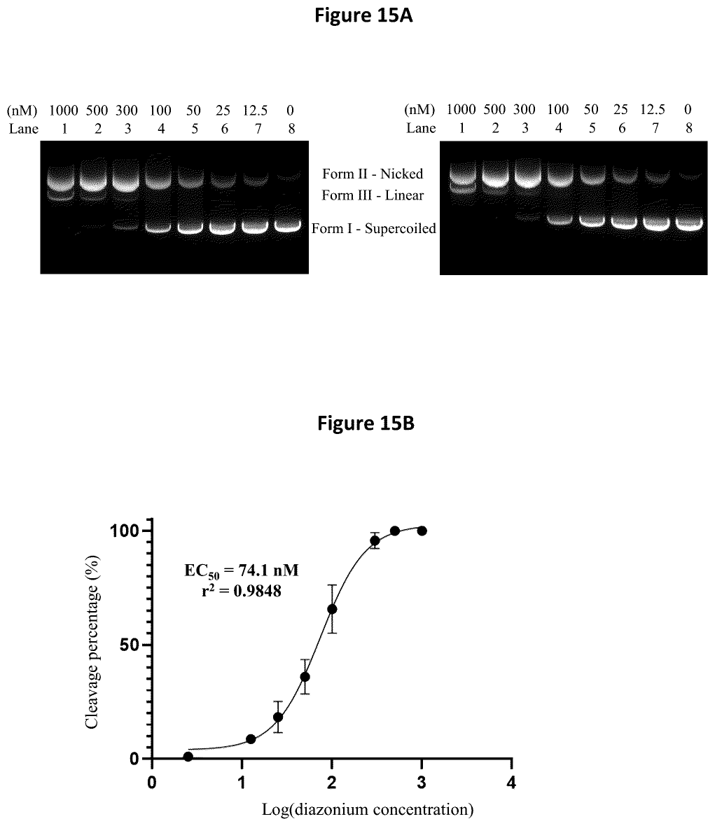

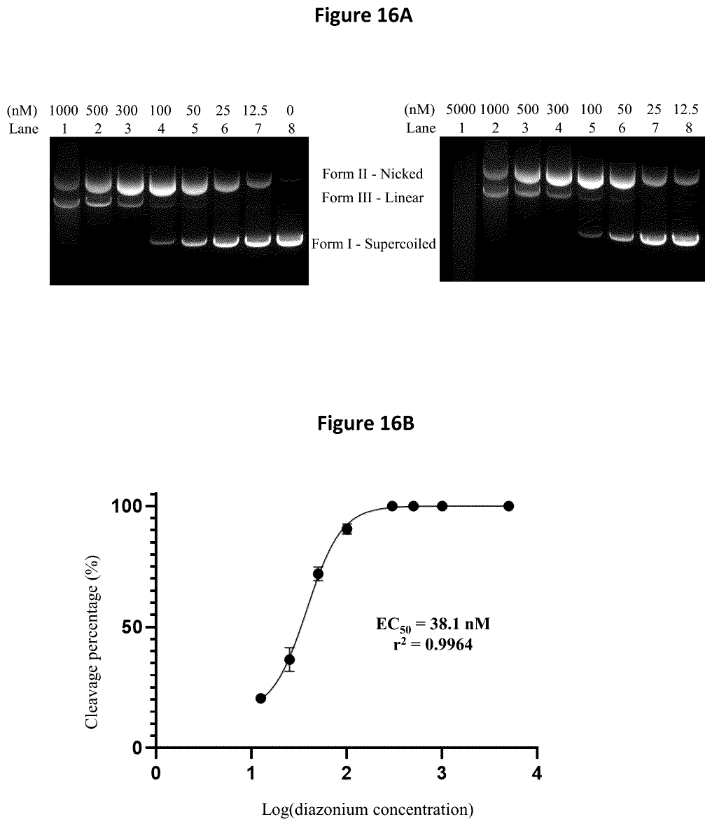

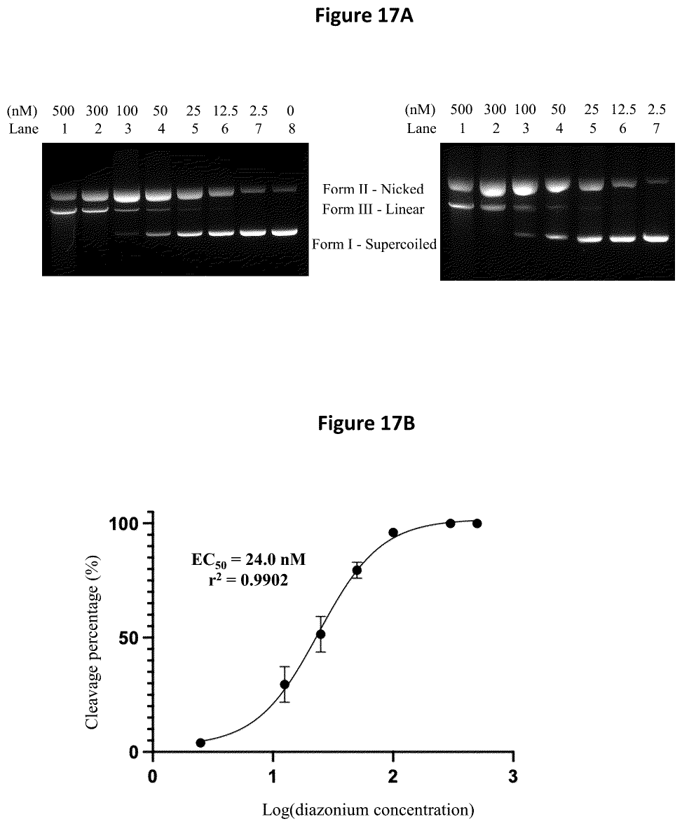

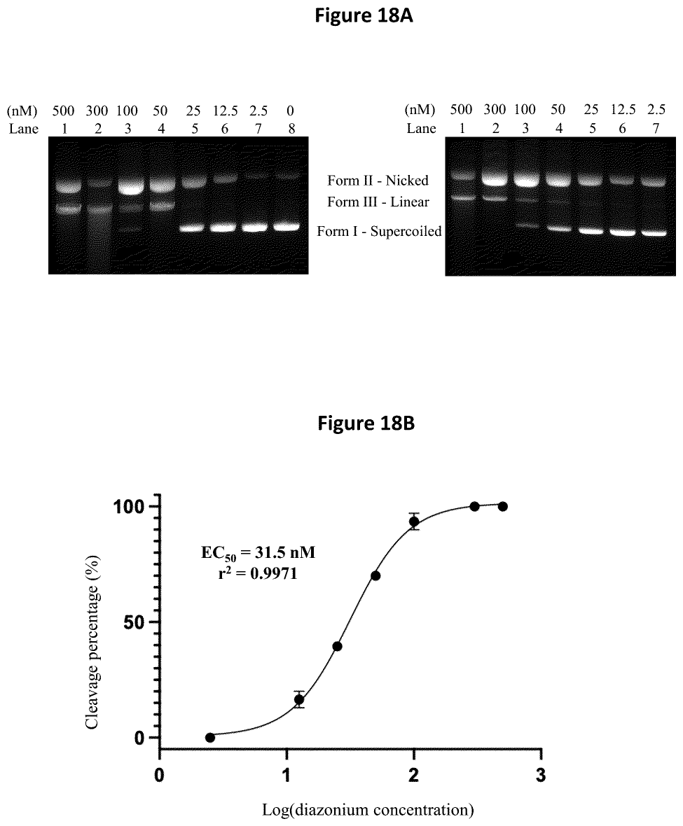

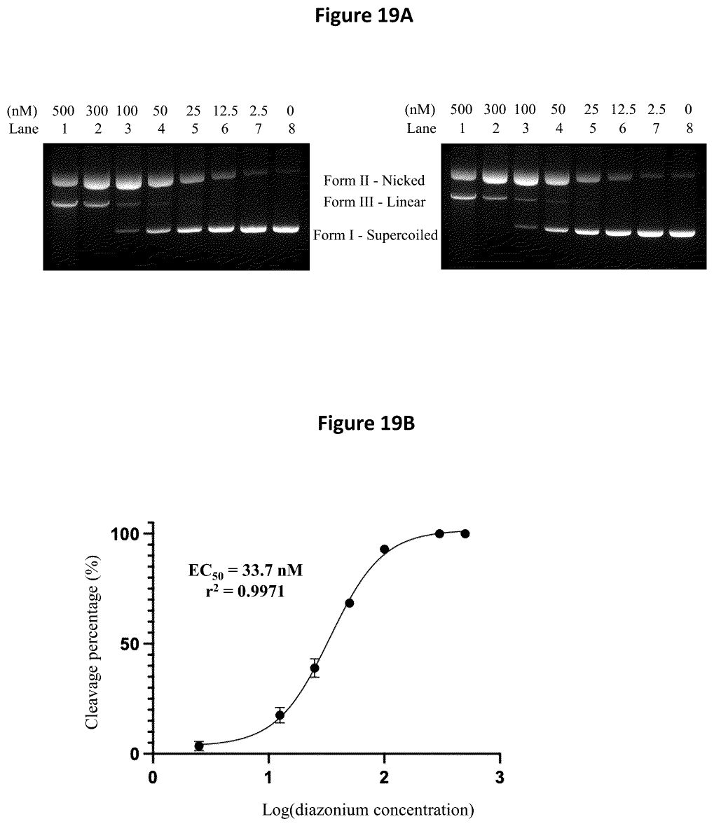

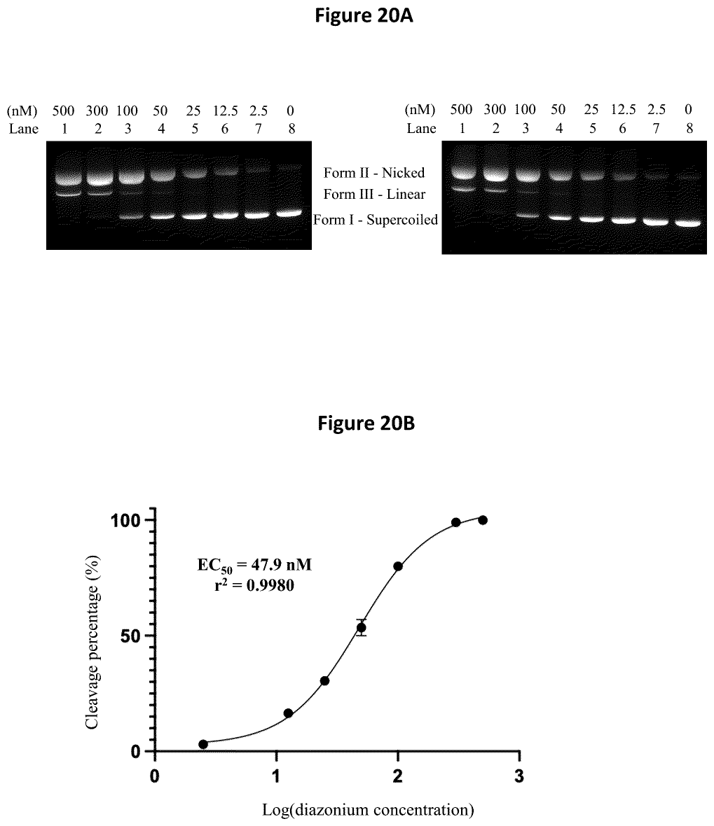

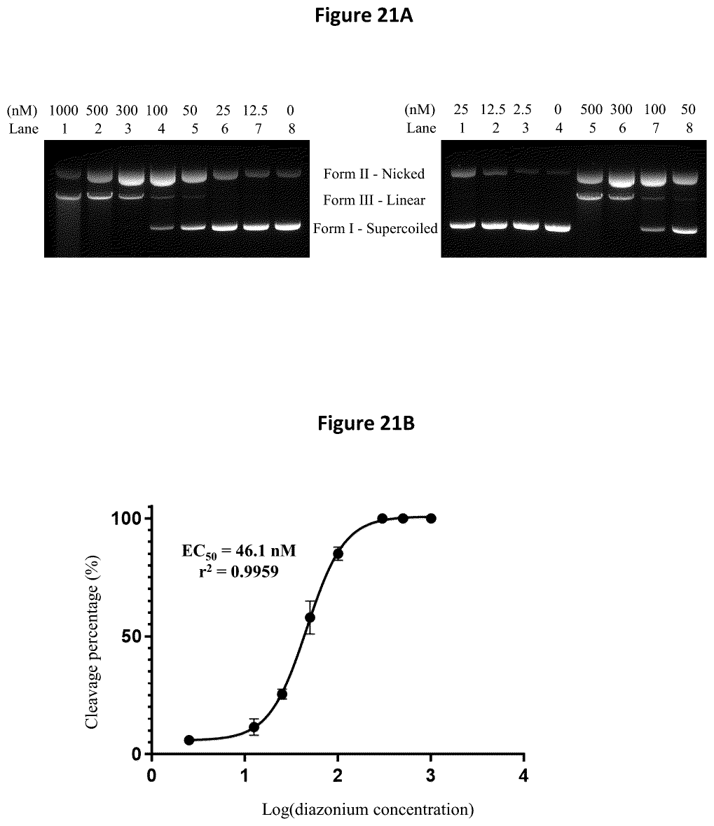

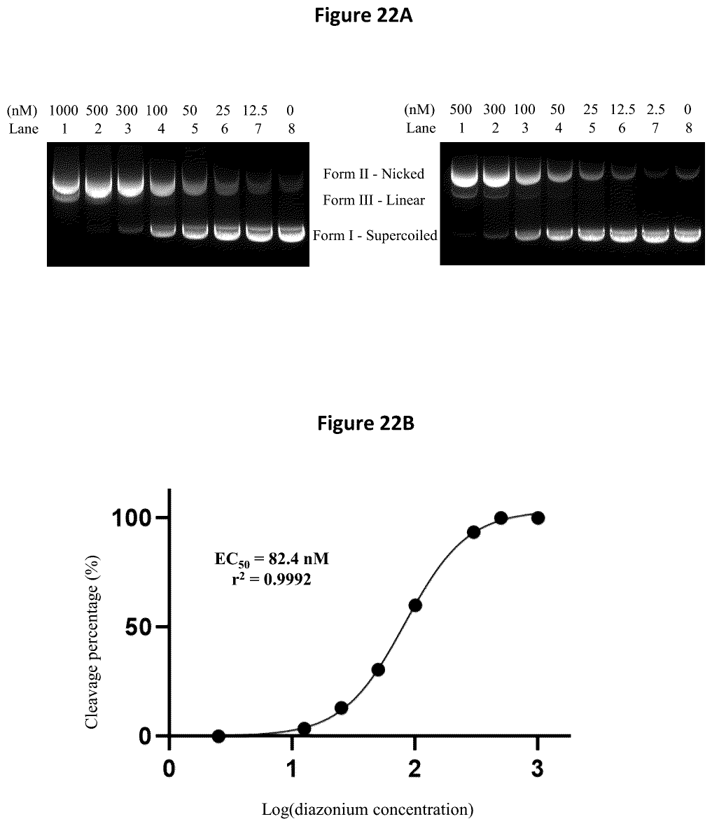

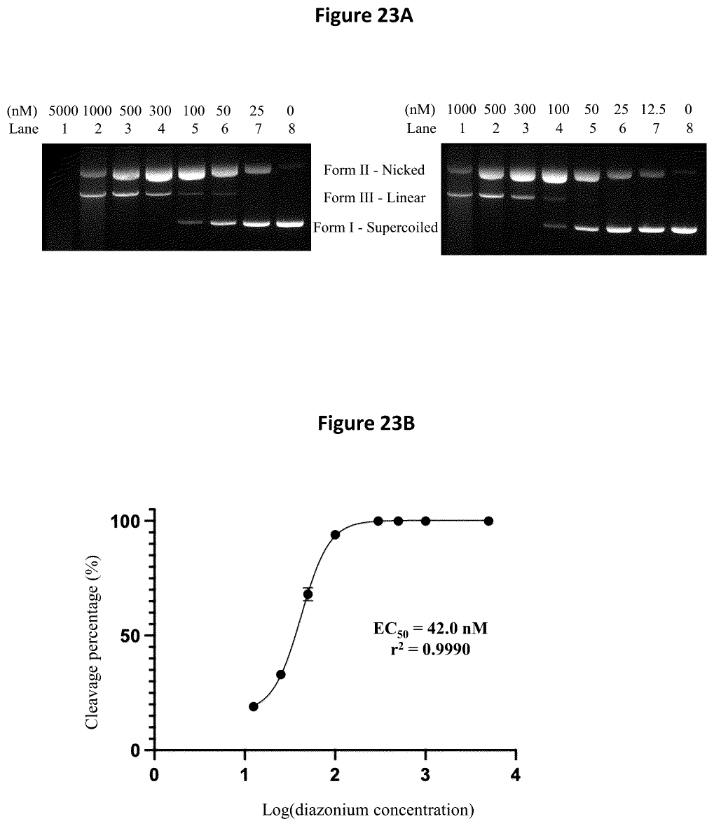

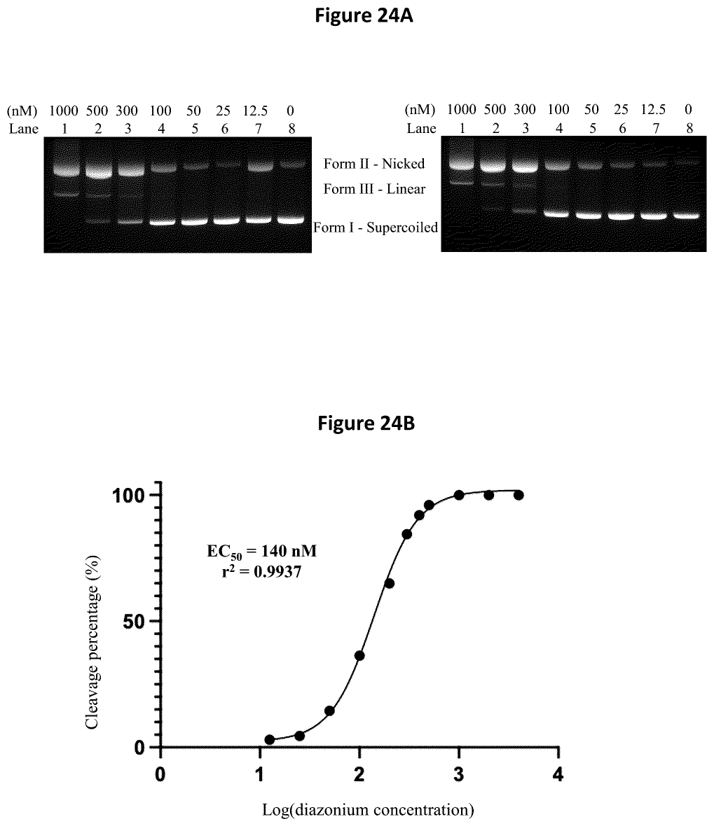

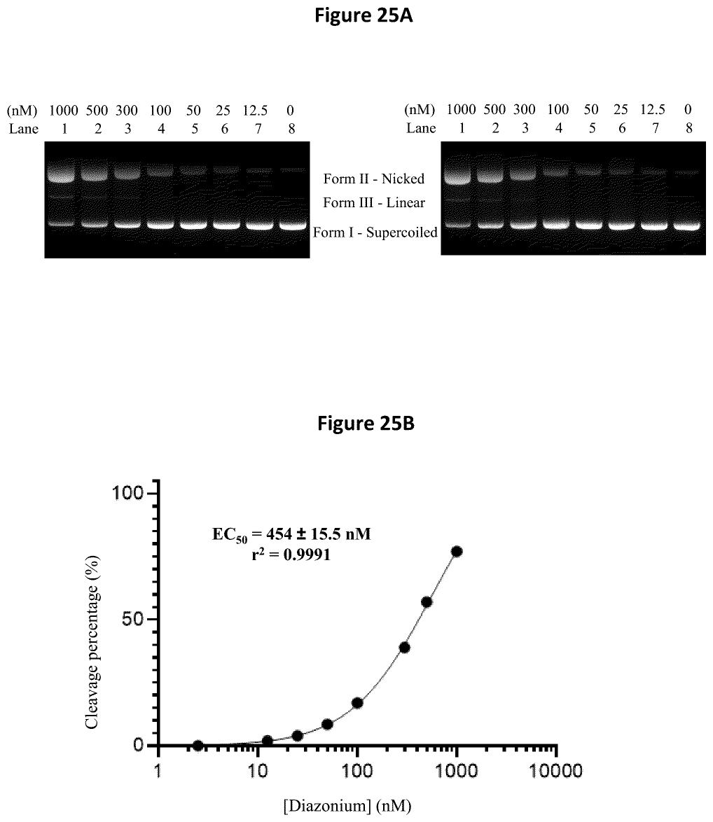

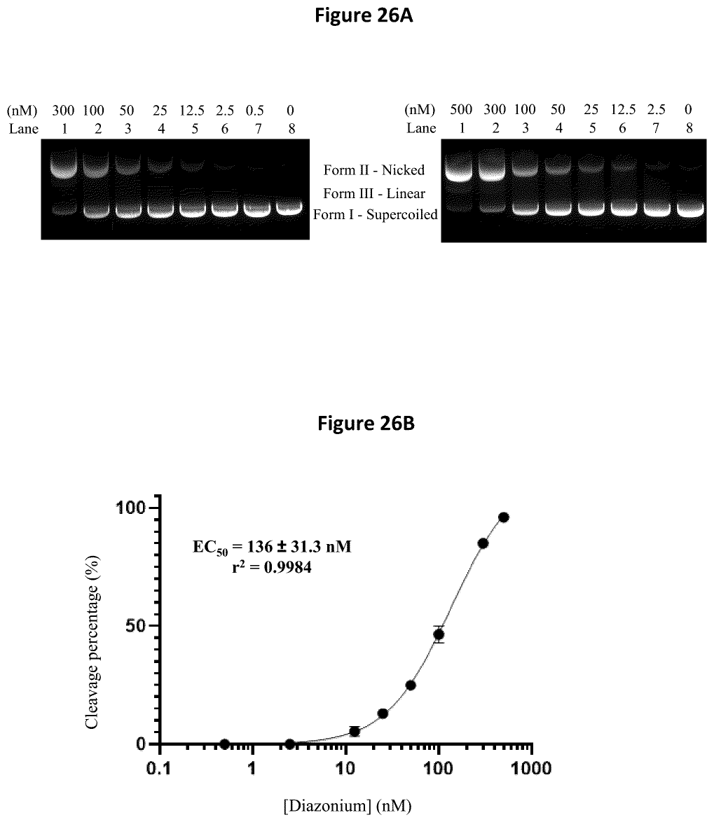

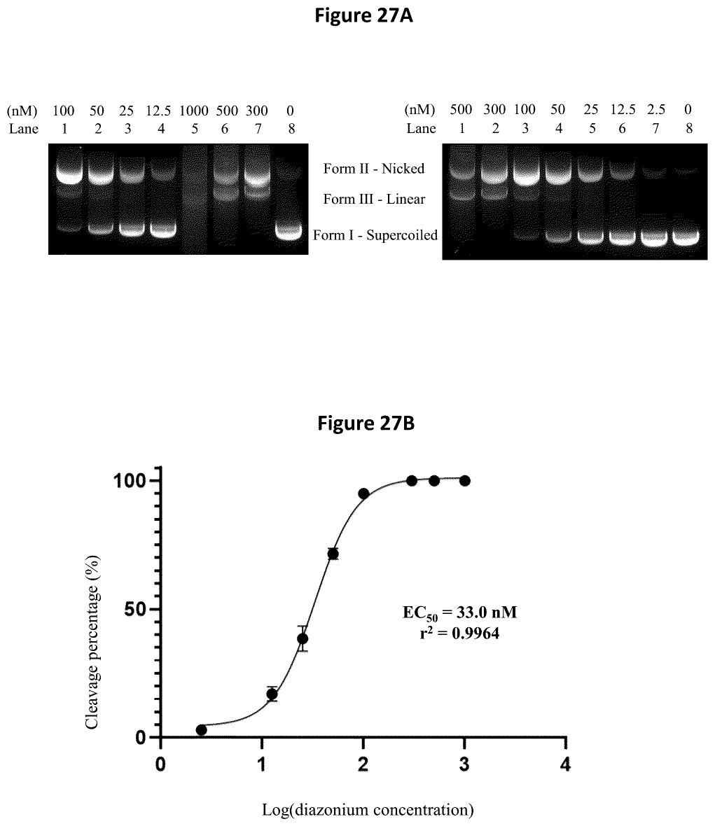

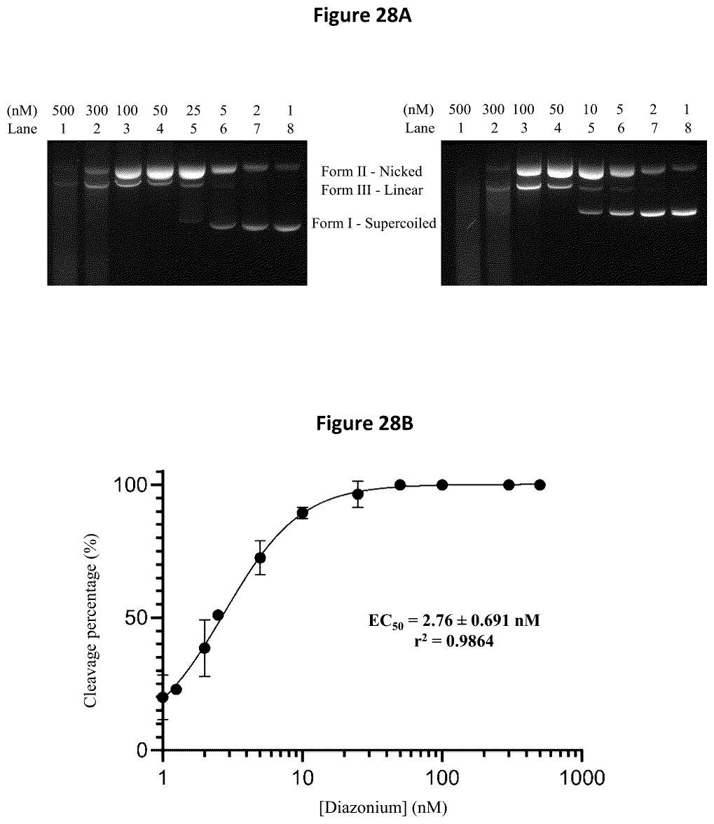

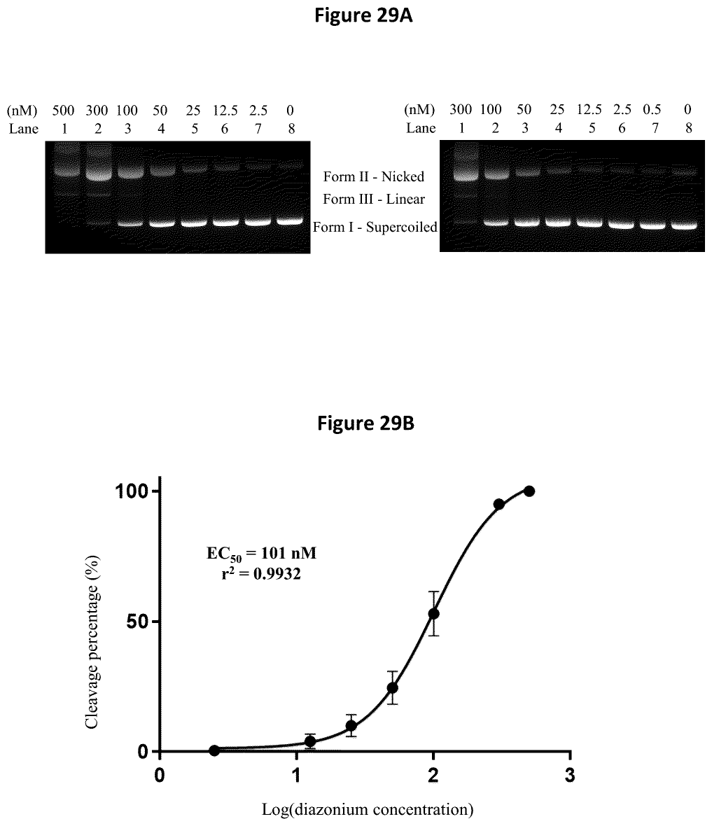

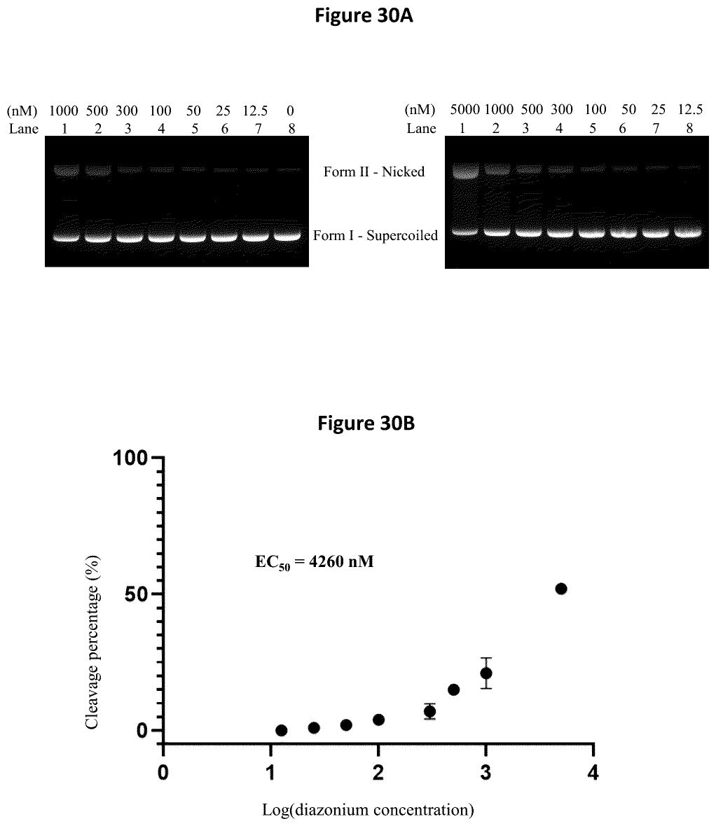

To determine potency, the EC₅₀ values for each diazonium salt were measured using a standardized 0.5 μg of DNA (40 μM/bp), visualization of the DNA conversion with agarose gel electrophoresis, and plotting of dose-response curves comparing the percentage of cleaved DNA against varying concentrations of the diazonium compounds. Agarose gel images and dose response curves of cleaved DNA percentages vs. concentrations of the diazonium salts are shown in FIGS. 1-30. The multidiazonium compounds with their EC₅₀ values are shown in Table 1 and the monodiazonium compounds with their EC₅₀ values are shown in Table 2.

Phenyl diazonium salt compound 1 exhibited limited activity with an EC₅₀ of 18.8 μM. The more electron-rich analogue compound 5 showed even lower activity compared to compound 1, while electron-deficient derivatives compounds 2-4 decreased the EC₅₀ by two orders of magnitude. This enhancement in potency by electron-withdrawing groups was also evident when comparing the naphthalene derivatives compounds 6 and 7, as well as the amide derivatives compounds 8 and 9.

To mimic the benzenoid diradical intermediate that arises from calicheamicin γ₁, bis-diazonium salts were explored. Benzene-1,4-bisdiazonium compound 11 displayed improved potency compared to monodiazonium compound 1. Similarly, naphthalene-1,5-bisdiazonium compound 10 outperformed compound 6. In analogue compounds 12-21, where the two diazonium groups are distributed across two different arene rings, connected via ketone, amide, ester, and ether linkages, there was a notable improvement in potency, with EC₅₀ values ranging from 18 nM to 74 nM. Notably, compound 12 was more effective than compound 15, indicating that the orientation and distance between the two radicals play a crucial role in their activity.

Varying the positions of the diazonium functional group on amide compounds 13 and 14 revealed similarly high activity compared to that of amide compound 12, suggesting an optimal distance between the diazo units. To investigate whether the amides engage in hydrogen bonding with DNA, thereby facilitating its coordination to DNA and subsequent cleavage, compound 21 with methyl protection was synthesized. Compound 21 exhibited good potency. The slightly higher EC₅₀ of 21, compared to those of 12, 13, and 14, may be attributed to the different dihedral angle of the two radicals, which is affected by the methylation of the amide. Ketone- and ether-linked diazonium salt compounds 18 and 20 showed similar, but slightly lower reactivity than their para-substituted counterparts, compounds 17 and 19, demonstrating that while the electronic effect is significant for reactivity in bisdiazonium compounds, it is less pronounced compared to the trend found with monodiazonium species.

(R) and (S) 1,1′-binaphthyl derivatives compounds 23 and 24 showed different potencies, suggesting that enantiomers are distinguished through molecular recognition via their interaction with DNA. The diazonium derivatives of acridine, known DNA intercalators (Tse, et al., 2004; Varakumar, et al., 2023), compounds 25 and 26 exhibited only modest potency, which are attributed to the misaligned orientation of the SOMOs (singly-occupied molecular orbitals) relative to the C—H bond at C5′ of the nucleotides.

Subsequently, the number of diazo units in the molecule were increased. Tri-diazonium compound 29 showed no notable improvement, possibly due to the extended distance between the diazo units. Tetra-diazonium salts compounds 27 and 28 displayed excellent potency. Specifically, 28 exhibited an EC₅₀ of 2.76 nM (2.14 ng/mL).

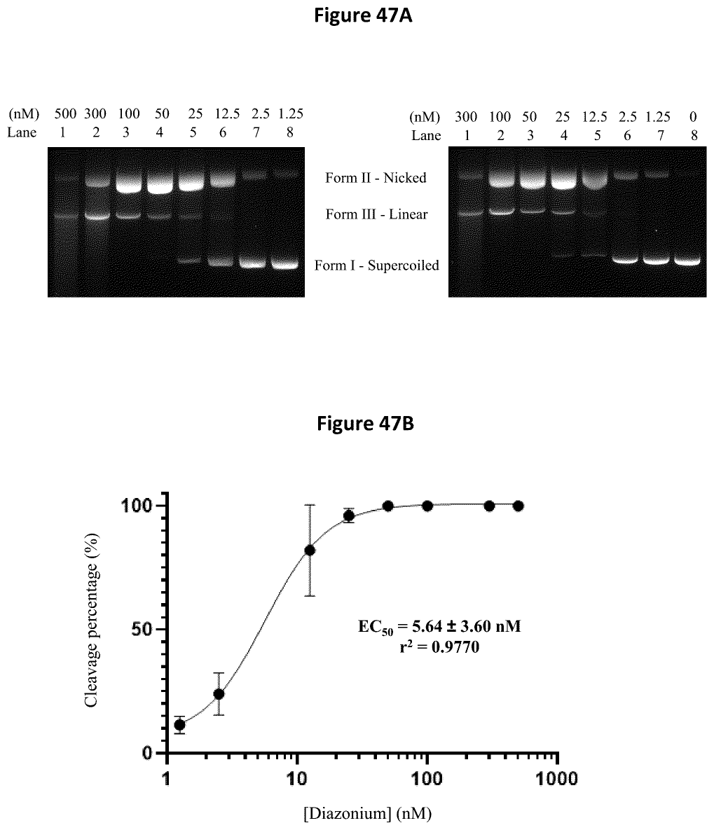

Compound 34 was synthesized, wherein an aniline is protected by acetate. Compound 34 showed excellent potency. Agarose gel images and dose response curve of cleaved DNA percentages vs. concentrations of compound 34 are shown in FIG. 47. The acetate protection suggests potential for conjugation of this potent diazonium compound with a linker, which could facilitate ADC development.

TABLE 1
Multidiazonium compounds.

| Diazonium | Reference | EC₅₀ value | | |
|---|---|---|---|---|
| compound | # | (nM) | SMILES | Structure |
| tetrakis(benzene-4- diazonium)methane tetrafluoroborate (Sengupta, et al., 1998) | 28 | 2.76 | N#[N+]C(C═C1)═CC═C1C(C2═ CC═C([N+]#N)C═C2)(C3═CC═ C([N+]#N)C═C3)C4═CC═C([N+] #N)C═C4•F[B−](F)(F)F•F[B−] (F)(F)F•F[B−](F)(F)F•F[B−] (F)(F)F | 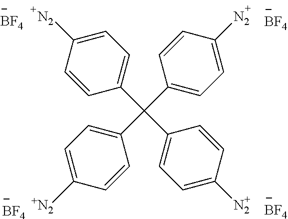 |
| 4,4′,4″-((4- acetamidophenyl) methanetriyl) tribenzenediazonium tetrafluoroborate | 34 | 5.64 | CC(NC1═CC═C(C(C2═CC═C ([N+]#N)C═C2)(C3═CC═C([N+]# N)C═C3)C4═CC═C([N+]#N)C═ C4)C═C1)═O•F[B−](F)(F)F•F[B−] (F)(F)F•F[B−](F)(F)F | 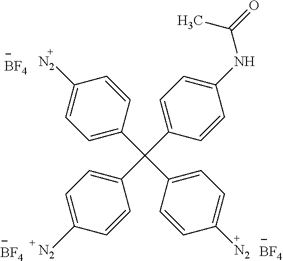 |
| N-(3-diazobenzene) 4-diazobenzamide tetrafluoroborate | 13 | 22.9 | O═C(NC1═CC([N+]#N)═CC═C1) C2═CC═C([N+]#N)C═C2•F[B−] (F)(F)F•F[B−](F)(F)F | 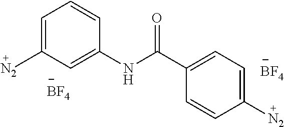 |
| benzophenone-4,4′- bisdiazonium tetrafluoroborate (Ma, et al., 2018) | 17 | 24.0 | O═C(C1═CC═C([N+]#N)C═C1) C2═CC═C([N+]#N)C═C2•F[B−] (F)(F)F•F[B−](F)(F)F | 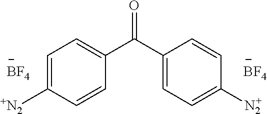 |
| N-(4-diazobenzene) 3-diazobenzamide tetrafluoroborate | 14 | 26.3 | O═C(NC1═CC═C([N+]#N)C═C1) C2═CC([N+]#N)═CC═C2•F[B−] (F)(F)F•F[B−](F)(F)F | 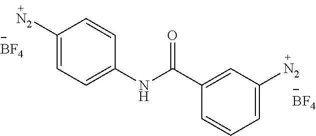 |
| benzophenone-3,3′- bisdiazonium tetrafluoroborate (Burke, et al., 1978) | 18 | 31.5 | O═C(C1═CC([N+]#N)═CC═C1) C2═CC═CC([N+]#N)═C2•F[B−] (F)(F)F•F[B−](F)(F)F | 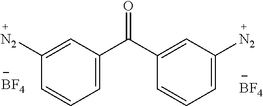 |
| tetrakis(benzene-4- diazonium)ethylene tetrafluoroborate (Anuradha, et al., 2016) | 27 | 33.0 | N#[N+]C(C═C1)═CC═C1/C (C2═CC═C([N+]#N)C═C2)═C (C3═CC═C([N+]#N)C═C3)/ C4═CC═C ([N+]#N)C═C4•F[B−] (F)(F)F•F[B−](F)(F)F•F[B−] (F)(F)F•F[B−](F)(F)F | 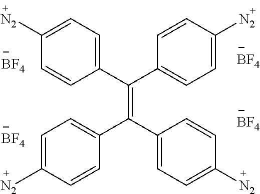 |
| di(benzene-4- diazonium)ether tetrafluoroborate (Russian Journal of Organic Chemistry, 1986) | 19 | 33.7 | N#[N+]C(C═C1)═CC═C1OC2═ CC═C([N+]#N)C═C2•F[B−] (F)(F)F•F[B−](F)(F)F | 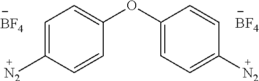 |
| N-(3-diazobenzene) 3-diazobenzamide tetrafluoroborate | 12 | 18.2 | O═C(NC1═CC([N+]#N)═CC═C1) C2═CC([N+]#N)═CC═C2•F[B−] (F)(F)F•F[B−](F)(F)F | 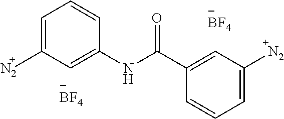 |
| (3-diazophenyl) 3- diazobenzoate tetrafluoroborate | 16 | 38.1 | O═C(OC1═CC([N+]#N)═CC═C1) C2═CC([N+]#N)═CC═C2•F[B−] (F)(F)F•F[B−](F)(F)F | 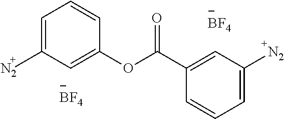 |
| (R)-[1,1′- binaphthalene]- 2,2′-bisdiazonium tetrafluoroborate (Parveen, et al., 2020) | 23 | 42.0 | N#[N+]C1═CC═C2C (C═CC═C2)═[C@] 1[C@]3═C([N+]#N) C═CC4═CC═CC═C43•F[B−] (F)(F)F•F[B−](F)(F)F | 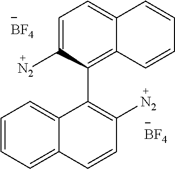 |
| N-(3- diazobenzene)-N- methyl-3- diazobenzamide tetrafluoroborate | 21 | 46.1 | O═C(N(C)C1═CC([N+] #N)═CC═C1)C2═CC([N+] #N)═CC═C2•F[B−] (F)(F)F•F[B−](F)(F)F | 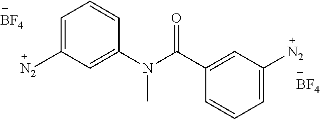 |
| di(benzene-3- diazonium)ether tetrafluoroborate (Tolstaya, et al., 1984) | 20 | 47.9 | N#[N+]C1═CC(OC2═CC([N+] #N)═CC═C2)═CC═C1•F[B−] (F)(F)F•F[B−](F)(F)F | 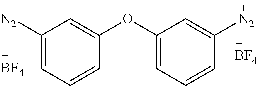 |
| N-(4-diazobenzene) 4-diazobenzamide tetrafluoroborate (Russian Journal of Organic Chemistry, 1967) | 15 | 74.1 | O═C(NC1═CC═C([N+] #N)C═C1)C2═CC═C([N+]#N) C═C2•F[B−] (F)(F)F•F[B−](F)(F)F | 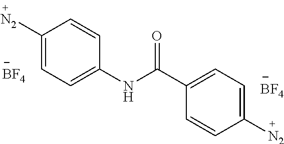 |
| N,N′-di(3- benzenediazonium) urea tetrafluoroborate | 22 | 82.4 | O═C(NC1═CC([N+]#N)═CC═C1) NC2═CC([N+]#N)═CC═C2•F [B−](F)(F)F•F[B−](F)(F)F | 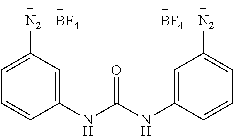 |
| N¹,N³,N⁵- tris(benzene-4- diazonium)benzene- 1,3,5- tricarboxamide tetrafluoroborate | 29 | 101 | F[B−](F)(F)F•O═C (NC1═CC═([N+] #N)C═C1)C2═CC(C(NC3═CC═C ([N+]#N)C═C3)═O)═CC (C(NC4═CC═C ([N+]#N)C═C4)═O)═C2•F [B−](F)(F)F•F[B−](F)(F)F | 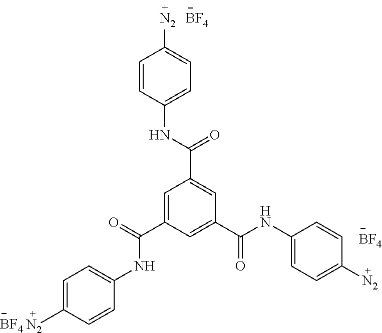 |
| naphthalene-1,5- bisdiazonium tetrafluoroborate (Effaty, et al., 2023) | 10 | 31.7 | N#[N+] C1═CC═CC2═C1C═CC═C2 [N+]#N•F[B−](F)(F)F•F[B−] (F)(F)F | 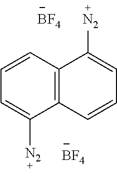 |
| ethidium bisdiazonium tetrafluoroborate (Whiteker, et al., 1991) | 26 | 136 | F[B−](F)(F)F•CC[N+] 1═C(C2═CC═CC═C2)C3═CC ([N+]#N)═CC═C3 C4═C1C═C(C═C4)[N+]#N•F [B−](F)(F)F•F[B−](F)(F)F | 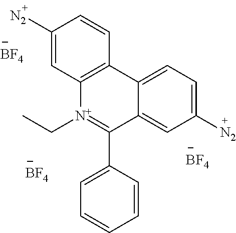 |
| (S)-[1,1′- binaphthalene]- 2,2′-bisdiazonium tetrafluoroborate (Parveen, et al., 2020) | 24 | 140 | N#[N+]C1═CC═C2C (C═CC═C2)═[C@@]1 [C@@]3═C([N+]#N) C═CC4═CC═CC═C43•F [B−](F)(F)F•F[B−](F)(F)F | 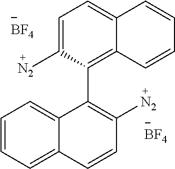 |
| acridine-3,5- bisdiazonium tetrafluoroborate | 25 | 454 | N#[N+]C1═CC2═[NH+]C3═C (C═CC([N+]#N)═C3) C═C2C═C1•F[B−] (F)(F)F•F[B−](F)(F)F•F[B−] (F)(F)F | 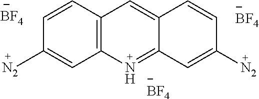 |
| benzene-1,4- bisdiazonium tetrafluoroborate (Marshall, et al., 2011) | 11 | 792 | N#[N+]C1═CC═C([N+]#N) C═C1•F[B−](F)(F)F•F[B−](F)(F)F | 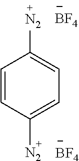 |
| methyl 3,5- bisdiazonium-1- benzoate tetrafluoroborate | 30 | 4260 | O═C(OC)C1═CC([N+]#N)═CC ([N+]#N)═C1•F[B−](F)(F)F•F[B−] (F)(F)F | 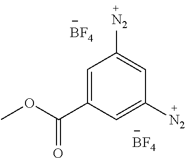 |

TABLE 2
Monodiazonium compounds.

| | Reference | EC₅₀ value | | |
|---|---|---|---|---|
| Diazonium compound | # | (nM) | SMILES | Structure |
| N-phenyl 3-diazobenzamide tetrafluoroborate | 9 | 289 | O═C (NC1═CC═CC═C1) C2═CC═CC([N+] #N)═C2•F[B−](F)(F)F | 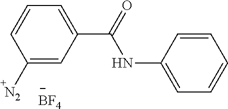 |
| 4-nitro-1-benzene-diazonium tetrafluoroborate (Pfaff, et al., 2021) | 2 | 371 | N#[N+]C1═CC═C ([N+]([O−])═O) C═C1•F[B−] (F)(F)F | 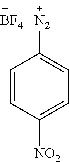 |
| 4-nitro-naphthalene-1- diazonium tetrafluoroborate (Bagal, et al., 1996) | 7 | 531 | N#[N+]C1═CC═C ([N+] ([O−])═O) C2═C1C═CC═C2•F [B−](F)(F)F | 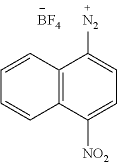 |
| N-(3-diazobenzene) benzamide tetrafluoroborate | 8 | 469 | O═C(C1═CC═CC═C1) NC2═CC═CC([N+] #N)═C2•F[B−](F)(F)F | 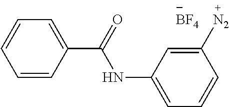 |
| 4-nitrile-benzene-1- diazonium tetrafluoroborate (Webb, et al., 2023) | 3 | 557 | N#[N+]C1═CC═C (C#N)C═C1•F[B−] (F)(F)F | 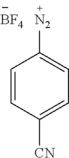 |
| methyl 4-benzoate-1- diazonium tetrafluoroborate (Ma, et al., 2018) | 4 | 571 | O═C(OC)C1═CC═C ([N+]#N)C═C1•F[B−] (F)(F)F | 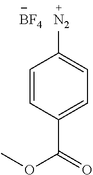 |
| naphthaene-1-diazonium tetrafluoroborate (Ma, et al., 2018) | 6 | 1080 | N#[N+] C1═CC═CC2═C1C═CC═C2•F [B−] (F)(F)F | 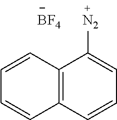 |
| benzenediazonium tetrafluoroborate (Pfaff, et al., 2021) | 1 | 18800 | N#[N+] C1═CC═CC═C1•F [B−](F)(F)F | 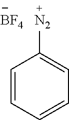 |
| 4-methoxy-benzene-1- diazonium tetrafluoroborate (Nie, et al., 2023) | 5 | 69800 | COC1═CC═C([N+]# N)C═C1•F[B−](F)(F)F | 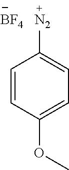 |

There was a significant dependence between the potency of the diazonium compound and the overall structure. In general, electron-deficient mono-diazonium salts exhibited EC₅₀ values between 290-530 nM. This contrasted with electron-rich mono-diazonium salts, which displayed an EC₅₀ value of up to 71,000 nM. Bis-diazonium compounds, which have two diazonium groups located on separate aryl rings linked by ether, ketone, amide, or ester bonds, achieved a much lower EC₅₀, ranging from 24-117 nM. Remarkably, when synthesizing the tetra-diazonium compound 28, an EC₅₀ value of 2.76 nM was observed. This EC₅₀ value is comparable to those of enediyne natural products, marking it as the lowest recorded EC₅₀ value for a synthetic small molecule that targets DNA.

### Example 3. Additional DNA-Cleaving Ability of Compounds 13 & 28

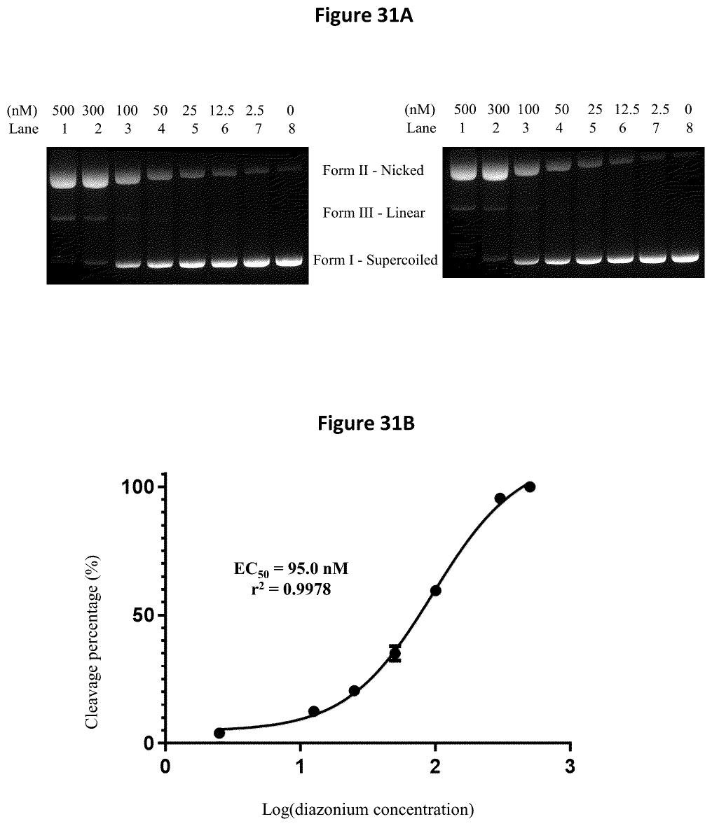

The DNA-cleaving ability of compound 13 was also tested under low-energy red light (660 nm). Agarose gel images and dose response curve of cleaved DNA percentage vs. concentrations of the diazonium salt compound 13 are shown in FIG. 31. An EC₅₀ value of 95.0 nM was observed for compound 13 under low-energy red light.

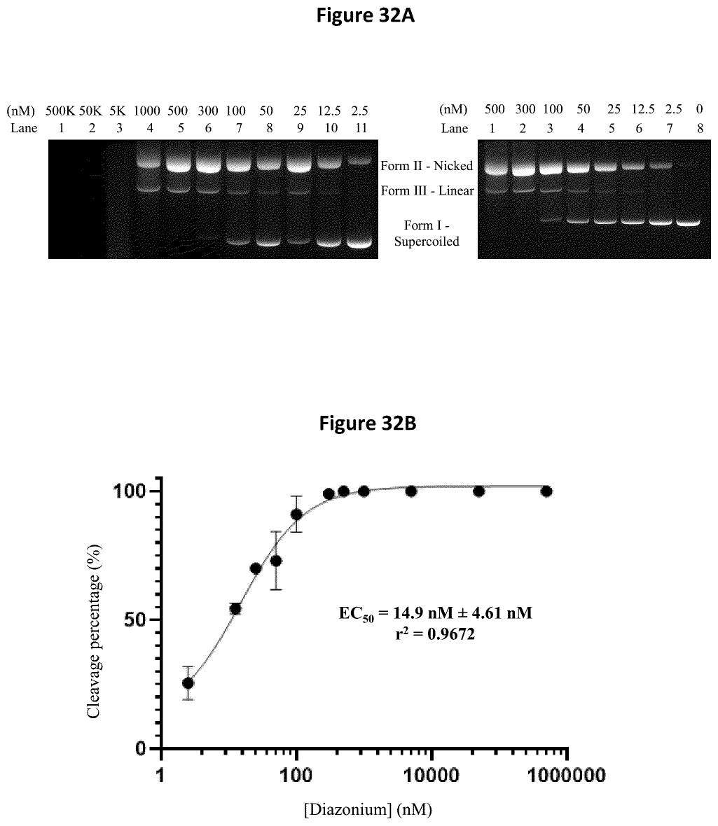

The DNA-cleaving ability of compound 28 was also tested under ambient light exposure. Agarose gel images and dose response curve of cleaved DNA percentage vs. concentrations of the diazonium salt compound 28 are shown in FIG. 32. An EC₅₀ value of 14.9 nM was observed for compound 28 under ambient light.

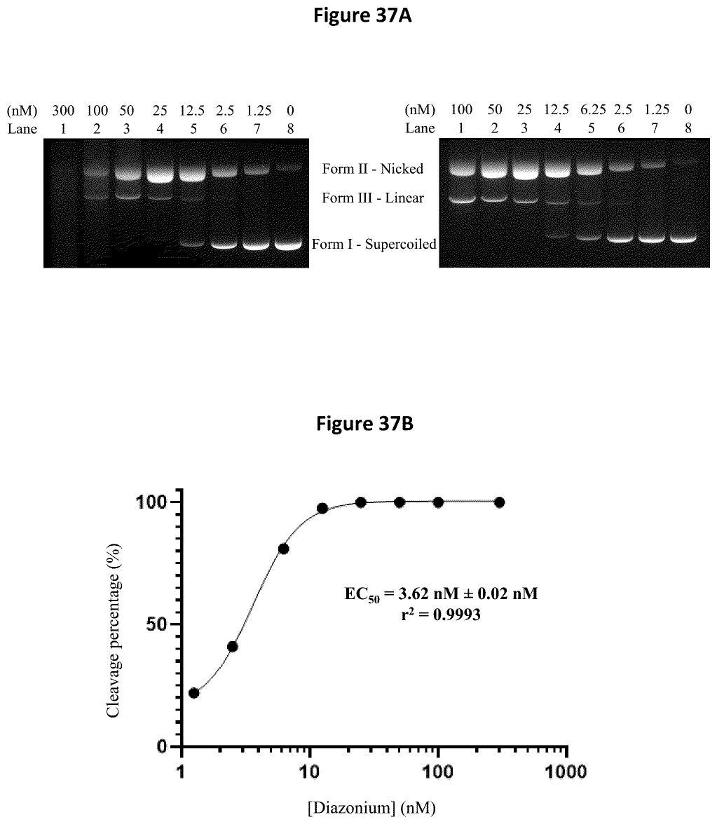

The DNA-cleaving ability of compound 28 was also tested under blue light (467 nm) exposure. Agarose gel images and dose response curve of cleaved DNA percentage vs. concentrations of the diazonium salt compound 28 are shown in FIG. 37. An EC₅₀ value of 3.62 nM was observed for compound 28 under blue light.

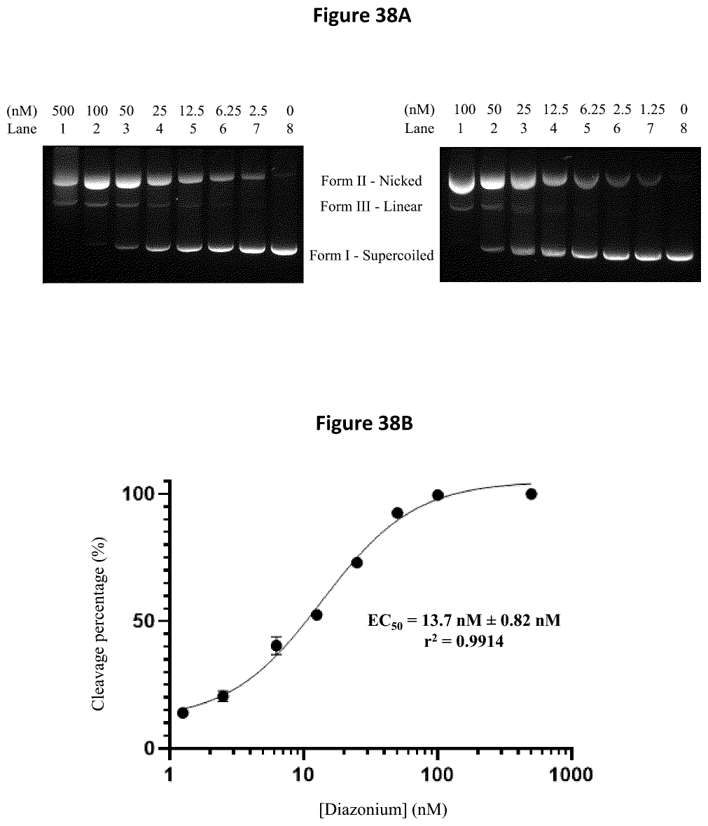

The DNA-cleaving ability of compound 28 was also tested under low-energy red light (660 nm) exposure. Agarose gel images and dose response curve of cleaved DNA percentage vs. concentrations of the diazonium salt compound 28 are shown in FIG. 38. An EC₅₀ value of 13.7 nM was observed for compound 28 under low-energy red light.

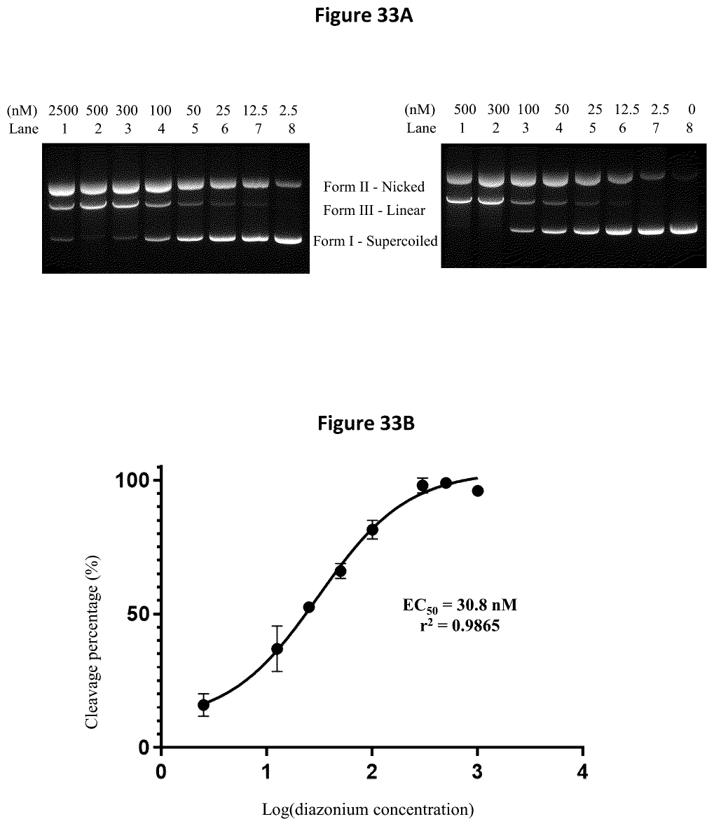

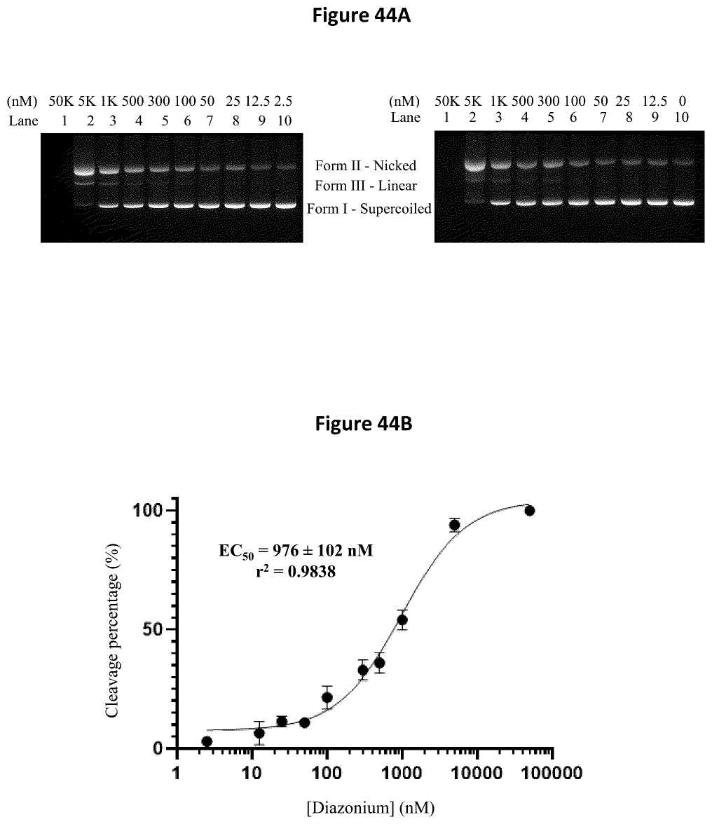

Further, the DNA-cleaving ability of compound 28 was tested with the presence of cysteine, a known reductant, in the dark. Agarose gel images and dose response curve of cleaved DNA percentage vs. concentrations of the diazonium salt compound 28 are shown in FIG. 33. An EC₅₀ value of 30.8 nM was observed for compound 28 in the presence of cysteine in the dark. This compares favorably to the DNA cleavage EC₅₀ value of about 1 μM (976 nM) performed in the dark without cysteine (FIG. 44).

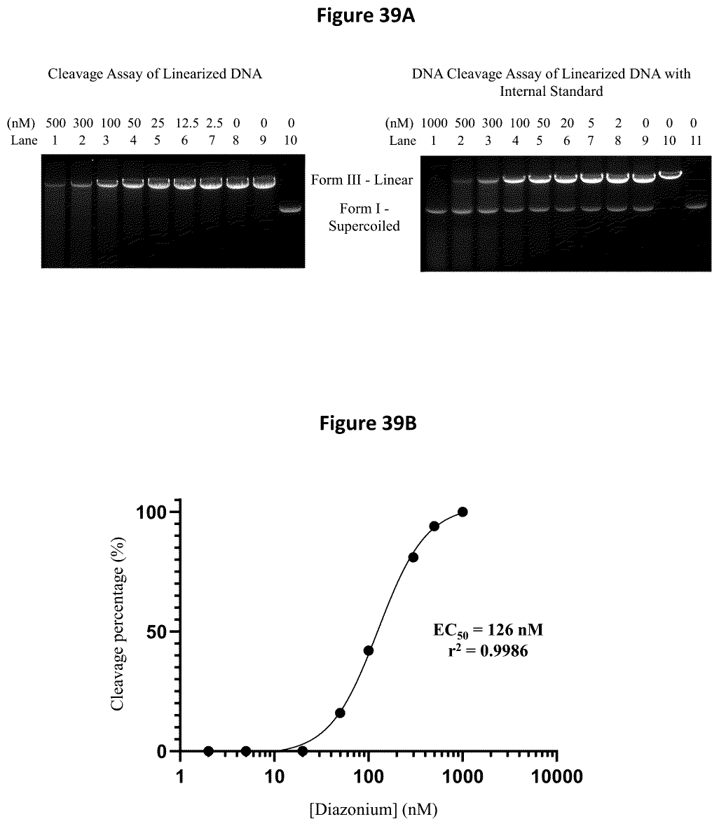

Moreover, the ability of compound 28 to cleave linearized double stranded DNA was tested under green light exposure. Agarose gel images and dose response curve of cleaved DNA percentage vs. concentrations of the diazonium salt compound 28 are shown in FIG. 39. An EC₅₀ value of 126 nM was observed for compound 28 on linearized double stranded DNA.

Compound 28 was effective in cleaving pre-linearized double-stranded DNA. Subjecting pre-linearized DNA pBR322 (Form III, 40 μM/bp) to compound 28 (irradiated with light of 525 nm in water for 1 hour) resulted in noticeable cleavage into low molecular weight fragments at concentrations of ≥100 nM (Lanes 1-3). Comparable cleavage profiles were observed by calicheamicin γ₁ and shishijimicin A (Zhang, et al., 2019). The need for higher concentrations of the diazonium salt to cleave linear DNA, compared to supercoiled DNA, may be attributed to certain molecular recognition with supercoiled DNA. Alternatively, the inherent strain in supercoiled DNA makes it more susceptible to cleavage than its linear form.

### Example 4. Synthesis and SAR of DNA Cleaving Ability of Diazonium Sugar Derivatives

To enhance potency and delineate a further SAR, the diazonium salts are synthesized with the addition of sugars. The sugars include, but are not limited to, glucose, galactose, mannose, or fructose. Additionally, a range of pyranoses and furanoses, and monosaccharides, disaccharides, and polysaccharides may be used. The general formula may resemble the structures as shown:

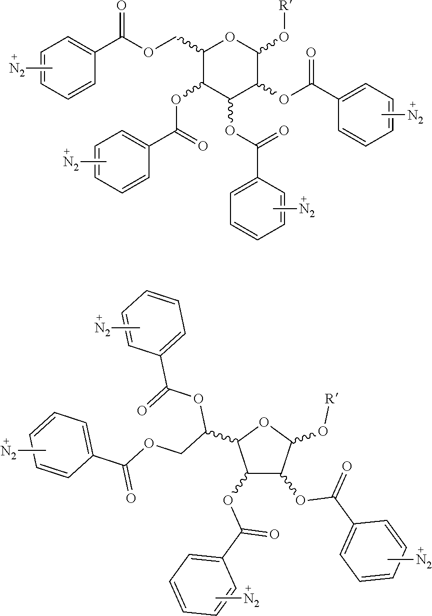

Monosaccharides were employed as scaffolds to host multiple diazonium moieties. The diazonium sugar derivatives created include alpha-glucose tetra-diazonium compound 31, alpha-galactose tetra-diazonium compound 32, and alpha-glucose tetra-diazonium compound 33. For synthesis of monosaccharide-based diazonium compounds, General Procedure 3 was performed as follows:

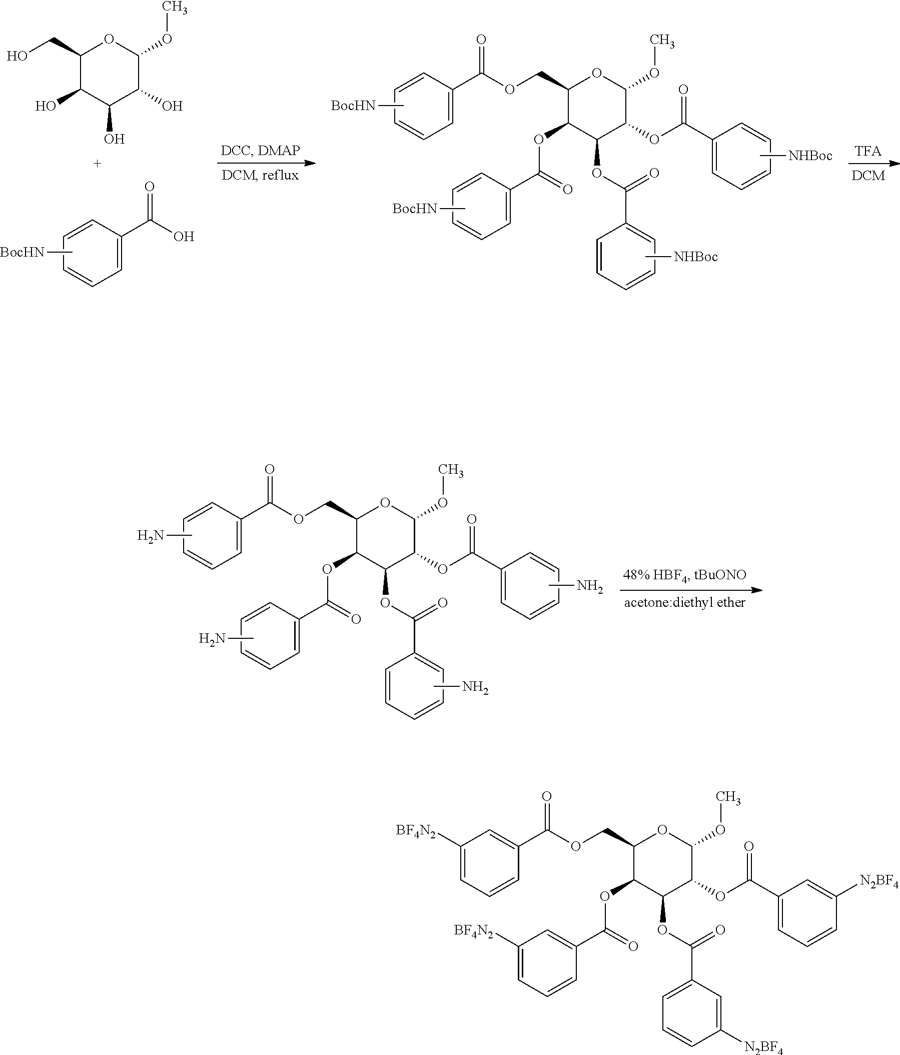

In a round bottom flask, the corresponding Boc-protected benzoic acid derivative (1.1 equiv. per hydroxyl), DMAP (1.0 equiv.) and DCC (dicyclohexylcarbodiimide) (1.1 equiv. per hydroxyl) were added to a solution of the corresponding sugar in DCM (0.1 M). The flask was sealed with a reflux condenser, purged with nitrogen, and allowed to stir at reflux for 16 hours. The mixture was allowed to cool to room temperature and an equivalent volume amount of diethyl ether was added. The solution was filtered through celite. The filtrate was concentrated and purified by flash column chromatography. The corresponding coupled product was deprotected by dissolving the sugar in DCM (0.5 M) followed by the addition of an equivalent volume of trifluoroacetic acid. The solution was stirred for 2 hours, neutralized with sodium bicarbonate (white precipitate was observed), and extracted in DCM and water. The organic layer was concentrated to afford the product, which was used without further purification. In a 20 mL glass vial, 0.100-1.00 mmol of corresponding amine was suspended in a mixture of 1 mL acetone, 1 mL diethyl ether, and 1 mL of a 48% aqueous solution of HBF₄. The vial was placed in an ice-brine bath and tert-butyl nitrite (6 equiv. per amine) was added dropwise, after which precipitate formed in the mixture. The slurry was allowed to stir in the ice-brine bath for 30 minutes. The solid was filtered off and washed with diethyl ether (2×10 mL). The collected diazonium salt was dried over vacuum for 5 minutes and collected without further purification.

The glycoside aryl amine precursor was prepared according to General Procedure 3 with methyl-ca-D-glucopyranoside as the starting glycoside and Boc-3-aminobenzoic acid. Using the glycoside aryl amine precursor (72 mg, 0.10 mmol), diazonium salt compound 31 was obtained as a white solid (97 mg, 91%). The NMR spectra show minor impurity. The compound was used without further purification.

The glycoside aryl amine precursor was prepared according to General Procedure 3 with methyl-α-D-galactopyranoside as the starting glycoside and Boc-3-aminobenzoic acid. Using the glycoside aryl amine precursor (100 mg, 0.15 mmol), diazonium salt compound 32 was obtained as a white solid (145 mg, 90%).

The glycoside aryl amine precursor was prepared according to General Procedure 3 with methyl-ca-D-glucopyranoside as the starting glycoside and Boc-4-aminobenzoic acid. Using the glycoside aryl amine precursor (91 mg, 0.14 mmol), diazonium salt compound 33 was obtained as a white solid (125 mg, 84%).

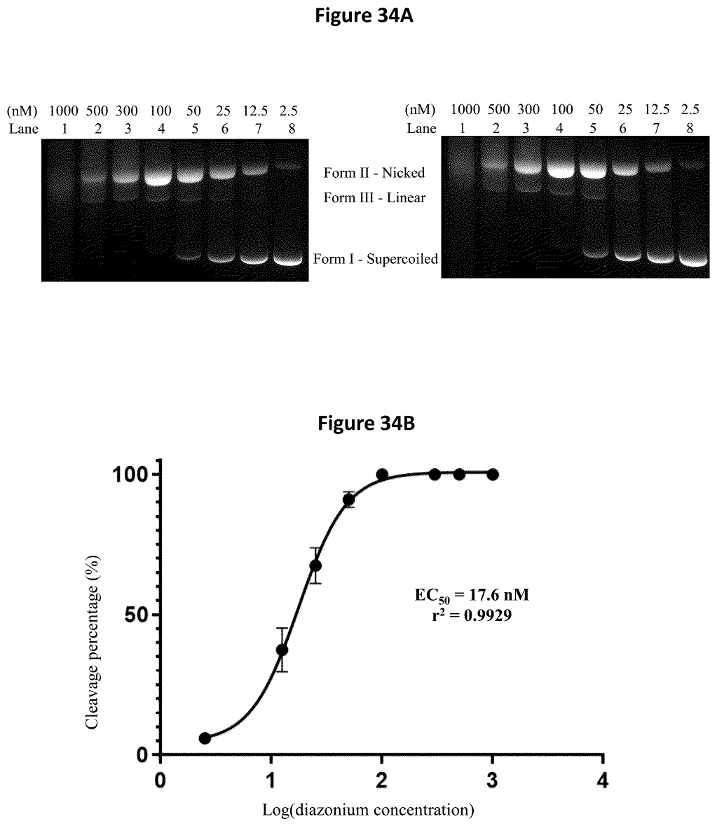

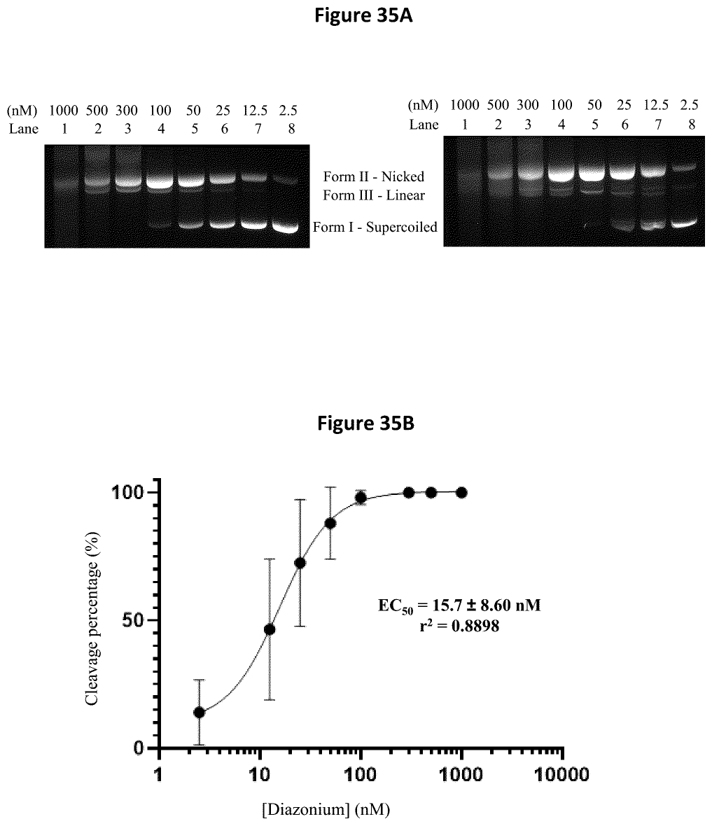

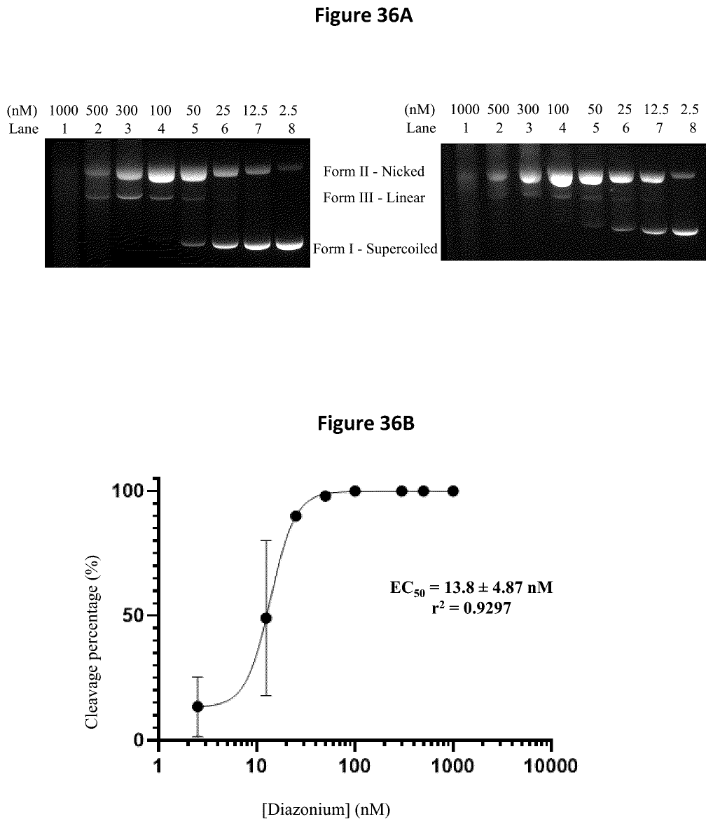

To examine the DNA-cleaving ability of the diazonium sugar derivatives, DNA was incubated with the diazonium sugar derivatives under green light irradiation (525 nm). The structures with their EC₅₀ values for DNA cleavage are shown in Table 3. Agarose gel images and dose response curves of cleaved DNA percentages vs. concentrations of the diazonium salts are shown in FIGS. 34-36.

Compounds 31, 32, and 33 demonstrated favorable potencies. The anomeric position in the carbohydrate framework serves as a handle for potentially linking to a minor-groove binder to enhance molecular recognition and achieve specificity (Ikemoto, et al., 1995). Moreover, this handle may serve to connect the diazonium compounds as a payload to an antibody in creation of ADCs.

Monosaccharide compound 33, with the diazonium units para to the ester of the sugar, displayed potency comparable to that of compounds 31 and 32.

TABLE 3
Diazonium sugar derivatives.

| Reference | EC₅₀ value | | |
|---|---|---|---|
| # | (nM) | SMILES | Structure |
| 31 | 17.6 | CO[C@@H]1[C@H](OC(C2═CC═CC ([N+]#N)═C2)═O) [C@@H](OC(C3═CC═CC ([N+]#N)═C3)═O)[C@H] (OC(C4═CC([N+]#N)═CC═C4)═O) [C@@H](COC(C5═CC([N+] #N)═CC═C5)═O)O1•F[B−](F)(F)F•F[B−] (F)(F)F•F[B−](F)(F)F•F[B−](F)(F)F | 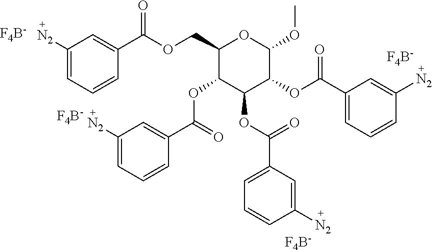 |
| 32 | 15.7 | CO[C@@H]1[C@H](OC(C2═CC═CC ([N+]#N)═C2)═O) [C@@H](OC(C3═CC═CC ([N+]#N)═C3)═O) [C@@H](OC(C4═CC ([N+]#N)═CC═C4)═O) [C@@H](COC(C5═CC ([N+]#N)═CC═C5)═O)O1•F[B−] (F)(F)F•F[B−](F)(F)F•F[B−](F)(F)F•F[B−] (F)(F)F | 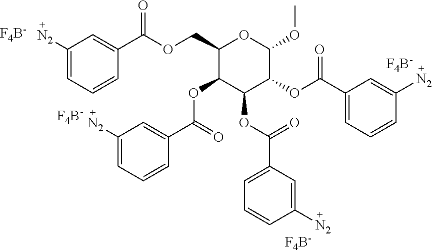 |
| 33 | 13.8 | CO[C@@H]1[C@H](OC(C2═CC═C([N+] #N)C═C2)═O)[C@@H](OC(C3═CC═C ([N+]#N)C═C3)═O) [C@H](OC(C4═CC═C ([N+]#N)C═C4)═O) [C@@H](COC(C5═CC═C ([N+]#N)C═C5)═O)O1•F [B−](F)(F)F•F[B−] (F)(F)F•F[B−](F)(F)F•F[B−](F)(F)F | 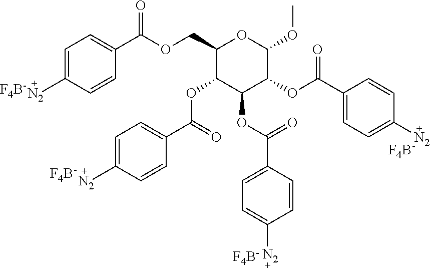 |

The characterization data for compound 31 was as follows: ¹H NMR (400 MHz, CD₃CN) δ 9.12-9.10 (m, 1H), 9.00-8.90 (m, 3H), 8.82 (dt, J=8.2, 1.4 Hz, 1H), 8.76-8.55 (m, 7H), 8.11 (t, J=8.2 Hz, 1H), 8.06-7.91 (m, 3H), 6.05 (dd, J=10.0, 9.1 Hz, 1H), 5.84 (dd, J=10.0, 9.1 Hz, 1H), 5.54 (dd, J=10.0, 3.6 Hz, 1H), 5.29 (d, J=3.6 Hz, 1H), 4.71-4.61 (m, 2H), 4.56 (dt, J=10.0, 3.0 Hz, 1H), 3.54 (s, 3H); ¹³C NMR (101 MHz, CD₃CN) δ 163.06, 163.01, 162.61, 162.47, 142.87, 142.87, 142.77, 142.65, 137.09, 136.98, 134.57, 134.11, 134.05, 133.55, 133.38, 133.36, 133.23, 117.11, 97.40, 73.46, 73.24, 71.03, 67.64, 64.63, 56.43; ¹⁹F NMR (471 MHz, CD₃CN) δ −151.32.

The characterization data for compound 32 was as follows: ¹H NMR (400 MHz, CD₃CN) δ 9.12 (t, J=1.9 Hz, 1H), 9.00 (t, J=1.9 Hz, 1H), 8.96 (t, J=1.9 Hz, 1H), 8.87 (t, J=1.9 Hz, 1H), 8.78-8.55 (m, 8H), 8.10-7.92 (m, 4H), 6.05 (dd, J=3.5, 1.2 Hz, 1H), 5.94 (dd, J=10.7, 3.5 Hz, 1H), 5.72 (dd, J=10.7, 3.6 Hz, 1H), 5.43 (d, J=3.6 Hz, 1H), 4.78-4.56 (m, 3H), 3.53 (s, 3H); ¹³C NMR (101 MHz, CD₃CN) δ 163.18, 162.88, 162.68, 162.55, 143.08, 142.77, 142.56, 137.24, 136.98, 136.93, 134.79, 134.16, 134.14, 134.01, 133.91, 133.55, 133.51, 133.41, 133.31, 133.23, 133.11, 117.10, 117.07, 117.05, 97.78, 71.92, 71.30, 70.71, 67.02, 64.64, 56.42; ¹⁹F NMR (471 MHz, CD₃CN) δ −151.10; IR (cm⁻¹): 3096, 2359, 2293, 1735, 1599, 1568, 1433, 1266, 1031, 966, 813, 746, 651, 541.

The characterization data for compound 33 was as follows: ¹H NMR (400 MHz, CD₃CN) δ 8.64-8.45 (m, 10H), 8.37-8.22 (m, 6H), 6.07 (t, J=9.6 Hz, 1H), 5.83 (t, J=9.6 Hz, 1H), 5.51 (dd, J=10.0, 3.6 Hz, 1H), 5.27 (d, J=3.6 Hz, 1H), 4.72-4.52 (m, 3H), 3.53 (s, 3H); ¹³C NMR (101 MHz, CD₃CN) δ 163.83, 163.78, 163.39, 163.33, 141.45, 141.45, 141.04, 140.93, 140.72, 133.78, 133.78, 133.67, 133.64, 133.12, 133.00, 132.97, 132.94, 120.09, 119.97, 119.92, 119.83, 97.41, 73.57, 73.35, 71.40, 67.58, 65.04, 56.39; ¹⁹F NMR (471 MHz, CD₃CN) δ −151.28; IR (cm⁻¹): 3113, 2294, 1732, 1603, 1508, 1417, 1265, 1034, 1011, 915, 855, 755, 687, 666, 578.

The diazonium sugar analogs provide insight into the role of stereochemistry and how the use of sugars affects the potency of the diazonium compounds.

### Example 5. Further Derivatization of Multi-Diazonium Compounds

To enhance potency and delineate a further SAR, the diazonium salts may be synthesized with the addition of diverse functional groups. Heterocycle-derived diazonium analogs may be synthesized and tested to discern the effects of heterocycles and their electronic effects. The heterocycle-derived diazonium analogs may include, but are not limited to:

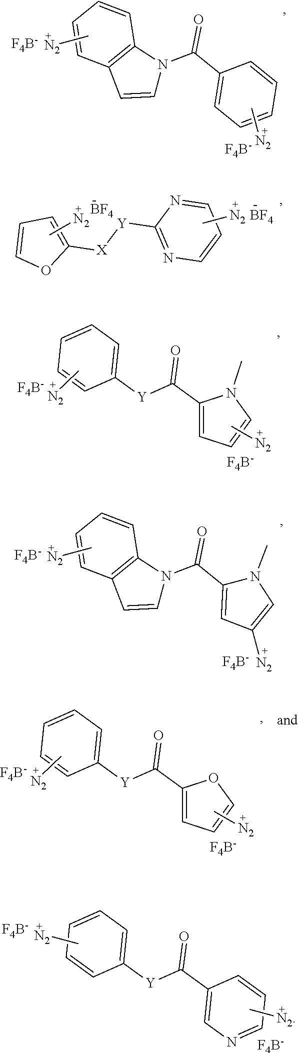

Similarly, to enhance potency and delineate a further SAR, substituted arene diazonium analogs may be synthesized and tested to discern the effects of substituents and their electronic effects. The substituted arene diazonium analogs may include, but are not limited to:

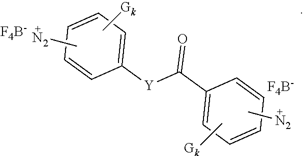

Additionally, the linkages between the diazonium-bearing aryl rings may be further manipulated, allowing added study of the electronic influence of the arenes and the spatial separation between the diazonium groups. The analysis of the thirty aryl diazonium analogs shown in Example 2 suggests that while the electronic properties of the arenes play a role, the distance between the diazonium groups and the torsion angles between the radicals might exert a more dominant effect. The manipulation of the linkages between the diazonium-bearing aryl rings is represented as follows:

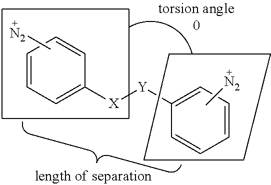

Additional diazonium derivatives may include triazabutadiene “pro-diazonium” molecules. These may also be referred to as “protected diazonium molecules”. These molecules mask the aryl diazonium compounds as triazabutadienes, that can release the aryl diazonium compounds selectively under specific triggers, e.g., low pH, irradiation, etc. Some triazabutadienes scaffolds may include, but are not limited to, the derivatives as follows:

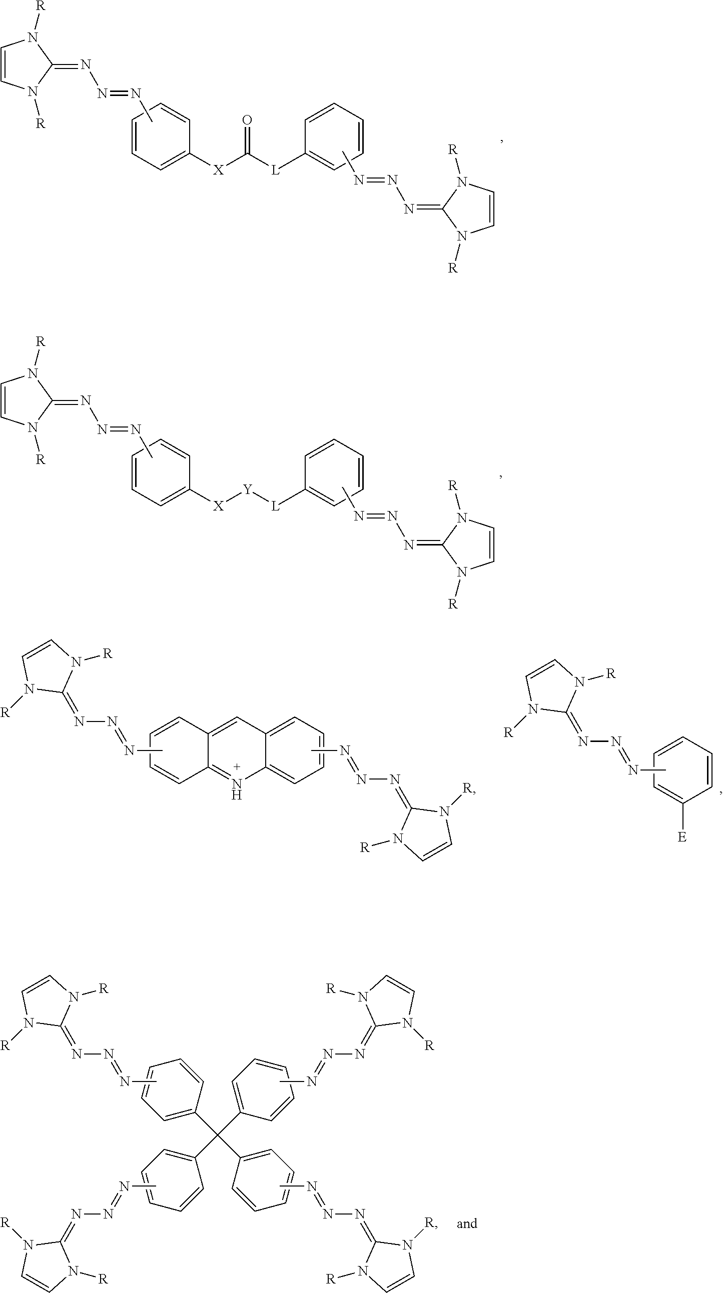

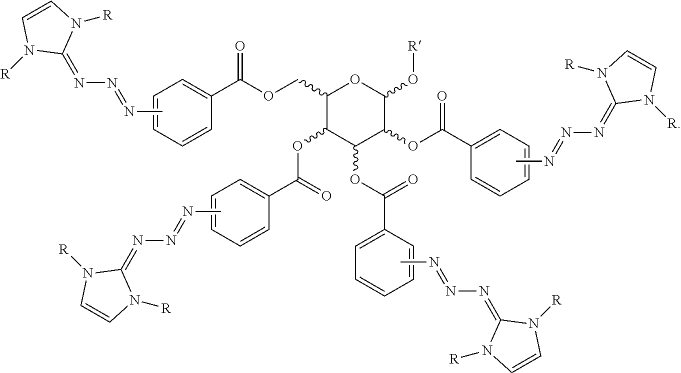

Moreover, to further determine SAR and increase potency of the diazonium compounds, peptide polymer derivatives may be synthesized and tested. These structures may include, but are not limited to, the following derivatives:

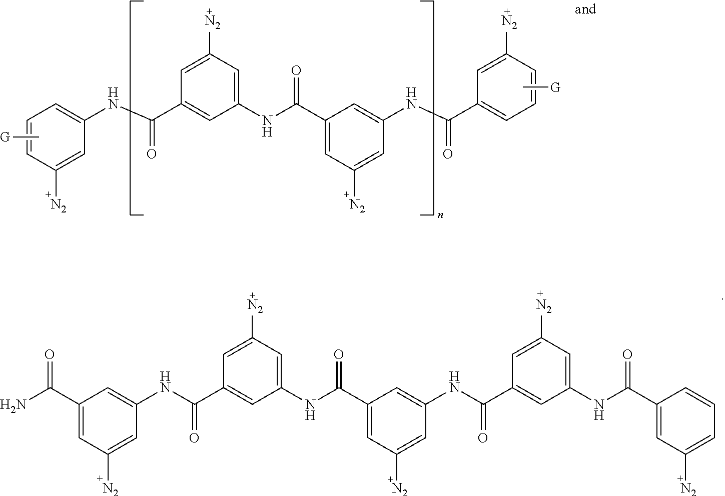

Further, to interpret the data and gain a deeper insight into these intricate factors, machine learning models may be employed for analysis. Deep learning models, notably T5Chem, are tailored for optimizing reactions (Lu, et al., 2022) and can be trained using the experimental dataset as described herein to identify the determining factors influencing performance. Recognizing that the deep learning language models are powerful few-shot learners (Brown, et al., 2020), T5Chem may be fine-tuned with the dataset as described herein. This effort may lead to proposals of more potent molecular candidates to synthesize and test experimentally.

### Example 6. Conjugates of Multi-Diazonium Compounds

To achieve sequence selectivity, diazonium compounds may be conjugated to minor groove binders. This approach would instill molecular recognition abilities to diazonium units, improving the binding affinity and the capability for sequence-specific recognition. Netropsin and distamycin A are renowned binders for AT-rich DNA sequences (Kopka, et al., 1985; Boger, et al., 2001). Diazonium compounds may be conjugated to distamycin A to demonstrate sequence selectivity in DNA cleavage. Diazonium-distamycin A conjugates may include, but are not limited to the following compounds:

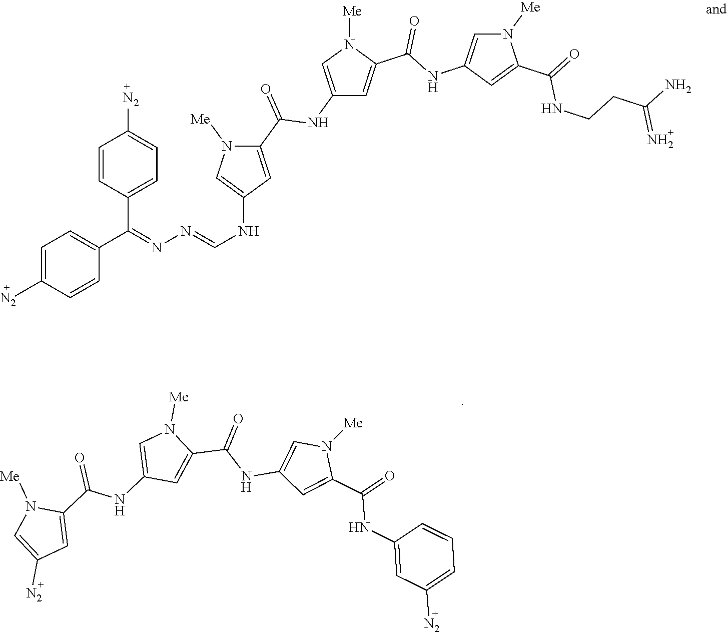

A series of synthesized diazonium-distamycin A conjugates would provide insight on the SAR, including key variables such as the balance between hydrophilicity and cell-permeability of the linker, the length of the linkage, and the strategic positioning of the diazonium compound on distamycin A. There is potential of distamycin A to act as a conduit between multiple diazonium groups.

Moreover, to improve the targeting of DNA, diazonium compounds may be conjugated to intercalators. Intercalators include ethidium bromide, anthracyclines, or acridine derivatives.

Further, antibody-drug conjugates (ADC) may be developed through conjugation of diazonium compounds to antibodies. Examples of antibody-drug conjugates comprising Trastuzumab conjugated to diazonium compounds may include, but are not limited to, the following compounds:

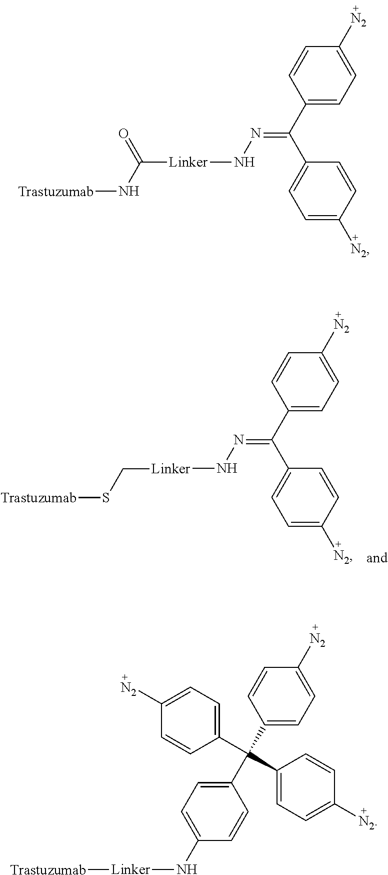

Antibodies provide a high level of specificity due to their capability for selective binding to target antigens. This approach would merge the specificity of antibodies with the cytotoxic capabilities of the potent diazonium salts (Lewis Phillips, et al., 2008). The humanized monoclonal antibody Trastuzumab is the focus, as Trastuzumab has been extensively studied and is clinically utilized due to its ability to selectively target Her-2, a receptor that is overexpressed in certain types of breast cancer cell lines (Cheng, et al., 2020). Upon binding with Trastuzumab, Her-2 is internalized by the cell, which would facilitate the delivery of the diazonium compounds into the cell (Lambert, et al., 2014).

Diazonium compounds may be conjugated to linkers to assess the effect on the diazonium potency. ADCs would be synthesized by conjugating Trastuzumab with potent diazonium compounds developed herein. Both Cys and Lys ligation technologies may be employed with various linkers, including Mal-PEG-NHS ester, DBCO-NHS ester, and SMCC, all of which are compatible with Trastuzumab (Copolovici, et al., 2014). The ADC's sensitivity and cytotoxicity may be assessed across a spectrum of cell lines that exhibit different levels of HER-2 expression, such as SK-Br-3 (human breast cancer) and Calu-3 (human lung carcinoma) (Lewis Phillips, et al., 2008; Copolovici, et al., 2014). The panel includes HMEC (human mammary epithelial cells) and NHEK (normal human epidermal keratinocytes) as sensitivity controls. The panel of cell lines would be incubated with different concentrations of Trastuzumab, Trastuzumab-diazonium conjugates, or the unattached diazonium. Post drug exposure, a clonogenic cell survival assay can be employed to construct dose-response curves, facilitating the quantification of IC₅₀ values and drug efficacies. These insights would provide critical insights into the therapeutic potential of these ADCs and guide optimization. Further, diazonium compounds may be conjugated on anti-CD33 and anti-CD22 antibodies to directly compare their performance with calicheamicin analogues in the same bioassays.

### Example 7. Investigation of Cytotoxicity of Multi-Diazonium Compounds

The cell permeability and cytotoxicity of the diazonium compounds may be investigated using a cell viability assay. If these compounds are cell-permeable, they could induce direct DNA damage in bacterial or cancer cells. Such permeability may be indicative of the bystander effect when these compounds are made into ADC drugs.

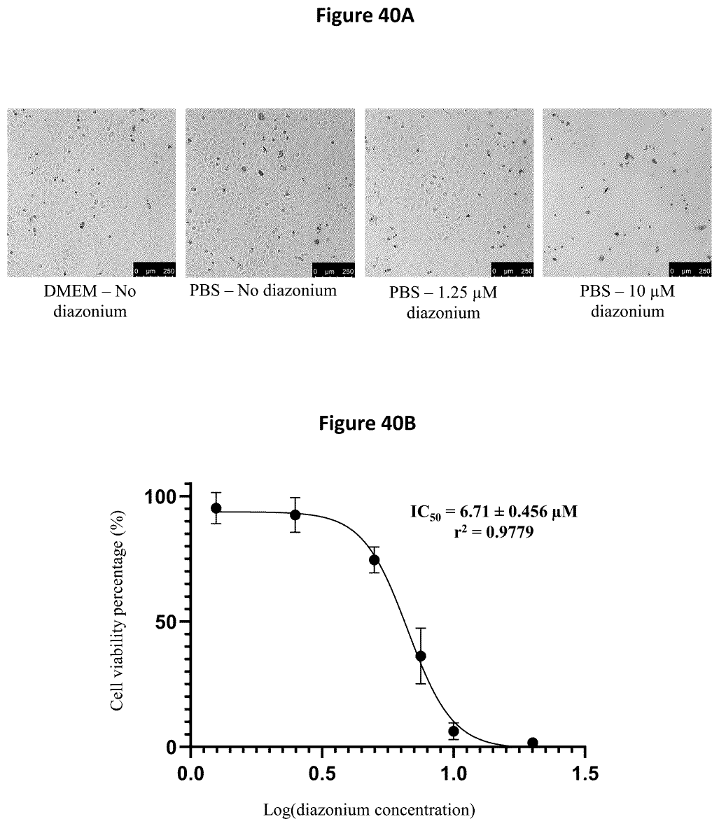

In a cell viability study testing cytotoxicity of the diazonium compounds, the antiproliferation activity of compound 28 was tested on live HeLa cells. Confocal microscopy images and dose response curve of cell viability percentage vs. concentrations of the diazonium compound 28 are shown in FIG. 40. The diazonium compound was effective in killing the cancer cells and the cell viability dose-response curve established an IC₅₀ of 6.71 μM (5.23 μg/mL).

The efficacy of the most potent diazonium compounds can be further assessed on a range of cell lines to establish a comprehensive understanding of their potential applications. These cell lines for testing include, but are not limited to, MCF-7, A549, and HEK293T cells, in addition to HeLa cells.

The compatibility of diazonium compounds with cell culture media can be evaluated to determine the stability of the compounds in media, such as DMEM, PBS, and RPMI-1640. The stability of the diazonium compounds may be enhanced by incorporating electron-donating groups or introducing steric bulk.

Alternative strategies can be implemented to enhance the ability of the diazonium compounds to traverse cellular membranes. One method would be designing cell-penetrating peptide mimetics (Copolovici, et al., 2014) that carry diazonium groups both as their payload and source of positive charges. These peptide mimetics with high density of positive charges could enter the cell via endocytosis, a process which involves wrapping the cell membrane around a charged species to form a vesicle.

### Example 8. Mechanistic Studies

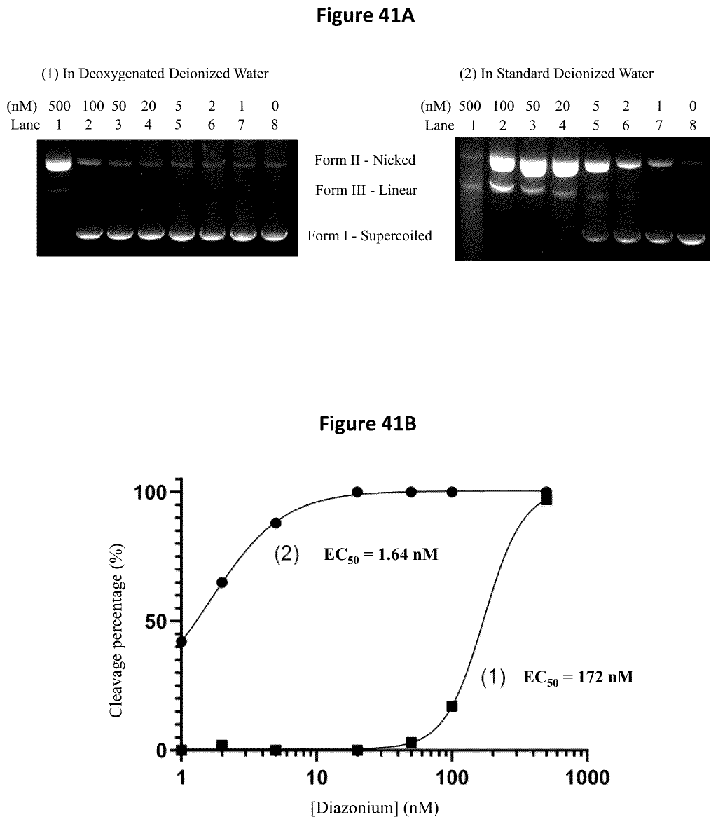

The ability of compound 28 to cleave DNA was tested in the presence of deoxygenated water. Agarose gel images and dose response curve of cleaved DNA percentage vs. concentrations of the diazonium salt compound 28 are shown in FIG. 41. Performing the reaction with degassed deionized water led to a significant 2-log scale decrease in activity compared to using standard deionized water (EC₅₀ values of 172 nM in degassed deionized water vs. 1.64 nM in standard deionized water), consistent with the known participation of O₂ in the cleavage process.

The effect of light irradiation on compound 28-mediated DNA cleavage was evaluated. Form I pBR322 DNA (40 μM/bp) was treated with compound 28 in water for 1 hour. This yielded DNA Form II and Form III. In darkness, the EC₅₀ value for DNA cleavage by compound 28 increased to 1 μM, substantiating the necessity of light for its activation. While a shorter wavelength of 467 nm provided activity (EC₅₀=3.62 nM) comparable to that of 525 nm (EC₅₀=2.76 nM), a longer wavelength (660 nm) (EC₅₀=13.7 nM) and ambient light (EC₅₀=14.9 nM) led to a slight decrease in reactivity.

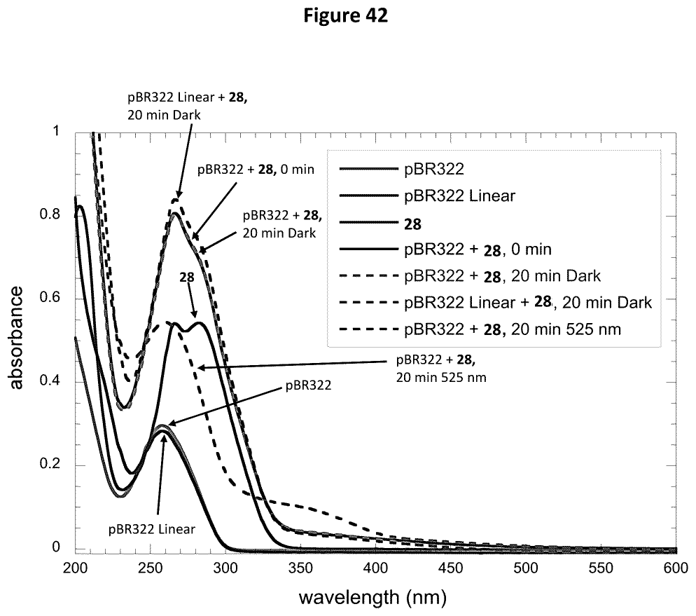

UV-Vis experiments were conducted to probe the interaction between diazonium compounds and DNA as shown in FIG. 42. Neither form of pBR322 (i.e., supercoiled or linear) nor diazonium compound 28 alone absorbed above 350 nm. Mixing supercoiled or linear DNA pBR322 with compound 28 gave similar absorbance spectra with an increase in absorption between 200-300 nm and a redshift in absorption bands compared to compound 28 alone, extending up to 500 nm. Incubating this mixture in darkness for 20 minutes did not alter the absorption spectra. In contrast, exposure to 525 nm light for 20 minutes significantly changed the spectrum. The increase in absorption intensity upon mixing DNA with compound 28 suggests that intercalation is unlikely, as intercalation typically results in decreased absorption due to quenching effects caused by DNA (Yu, et al., 1994). The redshift in the absorption bands supports an association between DNA and diazonium compounds, potentially explaining green light-promoted activation.

It is hypothesized that the ground-state association between supercoiled DNA and diazonium compounds facilitates inner-sphere charge transfer upon irradiation. This could lead to the reduction of diazonium salts by reductive components in DNA, such as guanine or adenine (Eₒₓ˜1 V vs. normal hydrogen electrode (NHE)) (Steenken, et al., 1997), although such charge transfer is typically unfavorable in an outer-sphere context determined by their redox potentials. Upon the extrusion of an N₂ molecule, the diazonium compounds generate aryl radicals that initiate HAT on nucleic acids. The resulting radical intermediates generated on polynucleic acids are subsequently trapped by O₂, leading to the oxidative cleavage of the DNA chain.

### REFERENCES

1. Halliwell, B.; Gutteridge, J. M. C. Free Radicals in Biology and Medicine. Oxford University Press 2015.
2. Zein, N.; Sinha, A. M.; McGahren, W. J.; Ellestad, G. A. Calicheamicin Y: an Antitumor Antibiotic That Cleaves Double-Stranded DNA Site Specifically. Science 1988, 240, 1198-1201.
3. Andrieux, C. P.; Pinson, J. The Standard Redox Potential of the Phenyl Radical/Anion Couple. J. Am. Chem. Soc. 2003, 125, 14801-14806.
4. (a) Hari, D. P.; Schroll, P.; Konig, B. Metal-Free, Visible-Light-Mediated Direct C—H Arylation of Heteroarenes with Aryl Diazonium Salts. J. Am. Chem. Soc. 2012, 134, 2958-2961. (b) Romero, N. A.; Nicewicz, D. A. Organic Photoredox Catalysis. Chem. Rev. 2016, 116, 10075-10166.
5. Hamann, P. R.; Hinman, L. M.; Hollander, I.; Beyer, C. F.; Lindh, D.; Holcomb, R.; Hallett, W.; Tsou, H.-R.; Upeslacis, J.; Shochat, D.; Mountain, A.; Flowers, D. A.; Bernstein, I. Gemtuzumab Ozogamicin, A Potent and Selective Anti-CD33 Antibody-Calicheamicin Conjugate for Treatment of Acute Myeloid Leukemia. Bioconjugate Chem. 2002, 13(1), 47-58.
6. Lee, M. D.; Dunne, T. S.; Chang, C. C.; Siegel, M. M.; Morton, G. O.; Ellestad, G. A.; McGahren, W. J.; Borders, D. B. Calicheamicins, a Novel Family of Antitumor Antibiotics.
4. Structure Elucidation of Calicheamicins β₁^{Br}, γ₁^{Br}, α₂^{I}, α₃^{I}, β₁^{I}, γ₁^{I}, and δ₁^{I}. J. Am. Chem. Soc. 1992, 114(3), 985-997.
7. (a) Smith, A. L.; Hwang, C. K.; Pitsinos, E.; Scarlato, G. R.; Nicolaou, K. C. Enantioselective Total Synthesis of (−)-Calicheamicinone. J. Am. Chem. Soc. 1992, 114, 3134-3136. (b) Nicolaou, K. C.; Hummel, C. W.; Pitsinos, E. N.; Nakada, M.; Smith, A. L.; Shibayama, K.; Saimoto, H. Total Synthesis of Calicheamicin γ₁^{I}. J. Am. Chem. Soc. 1992, 114(25), 10082-10084.
8. Galm, U.; Hager, M. H.; Van Lanen, S. G.; Ju, J.; Thorson, J. S.; Shen, B. Antitumor Antibiotics: Bleomycin, Enediynes, and Mitomycin. Chem. Rev. 2005, 105, 739-758.
9. Tong, J. T. W.; Harris, P. W. R.; Brimble, M. A.; Kavianinia, I. An Insight into FDA Approved Antibody-Drug Conjugates for Cancer Therapy. Molecules 2021, 26, 5847.
10. Abe, M. Diradicals. Chem. Rev. 2013, 113, 7011-7088.
11. Jones, R. R.; Bergman, R. G. p-Benzyne. Generation as an Intermediate in a Thermal Isomerization Reaction and Trapping Evidence for the 1,4-Benzenediyl Structure. J. Am. Chem. Soc. 1972, 94, 660-661.
12. Nicolaou, K. C.; Dai, W. M. Chemistry and Biology of the Enediyne Anticancer Antibiotics. Angei. Chem. Int. Ed. Engl. 1991, 30, 1387-1416.
13. Lee, M. D.; Ellestad, G. A.; Borders, D. B. Calicheamicins: Discovery, Structure, Chemistry, and Interaction with DNA. Acc. Chem. Res. 1991, 24, 235-243.
14. Zhang, H.; Li, R.; Ba, S.; Lu, Z.; Pitsinos, E. N.; Li, T.; Nicolaou, K. C. DNA Binding and Cleavage Modes of Shishijimicin A. J. Am. Chem. Soc. 2019, 141, 7842-7852.
15. Nicolaou, K. C.; Ogawa, Y; Zuccarello, G.; Kataoka, H. DNA Cleavage by a Synthetic Mimic of the Calicheamicin-Esperamicin Class of Antibiotics. J. Am. Chem. Soc. 1988, 110, 7247-7248.
16. Arya, D. P.; Warner, P. M.; Jebaratnam, D. J. Development of new DNA-binding and cleaving molecules: Design, synthesis and activity of a bisdiazonium salt. Tetrahedron Lett. 1993, 34, 7823-7826.
17. Arya, D. P.; Jebaratnam, D. J. Towards the development of non-enediyne approaches for mimicking enediyne chemistry: Design, synthesis and activity of a 1,4-bisdiazonium compound. Tetrahedron Lett. 1995, 36, 4369-4372.
18. Basak, A.; Mandal, S.; Bag, S. S. Chelation-Controlled Bergman Cyclization: Synthesis and Reactivity of Enediynyl Ligands. Chem. Rev. 2003, 103, 4077-4094.
19. Mohler, D. L.; Gray Coonce, J.; Predecki, D. Photoinduced DNA cleavage by benzenediradical equivalents: 1,3- and 1,4-Bis(dicarbonylcyclopentadienyliron)benzene. Bioorg. Med. Chem. Lett. 2003, 13, 1377-1379.
20. Ylijoki, K. E. O.; Lavy, S.; Fretzen, A.; Kündig, E. P.; Berclaz, T.; Bernardinelli, G.; Besnard, C. A Synthetic and Mechanistic Investigation of the Chromium Tricarbonyl-Mediated Masamune-Bergman Cyclization. Direct Observation of a Ground-State Triplet p-Benzyne Biradical. Organometallics 2012, 31, 5396-5404.
21. Bhattacharya, P.; Basak, A.; Campbell, A.; Alabugin, I. V. Photochemical Activation of Enediyne Warheads: A Potential Tool for Targeted Antitumor Therapy. Mol. Pharmaceutics 2018, 15, 768-797.
22. Arya, D. P. Diazo and Diazonium DNA Cleavage Agents: Studies on Model Systems and Natural Product Mechanisms of Action Top. Heterocycl. Chem. 2006, 2, 129-152.
23. Lu, J.; Zhang, Y Unified Deep Learning Model for Multitask Reaction Predictions with Explanation. J. Chem. Inf. Model. 2022, 62, 1376-1387.
24. Brown, T. B.; Mann, B.; Ryder, N.; Subbiah, M.; Kaplan, J.; Dhariwal, P.; Neelakantan, A.; Shyam, P.; Sastry, G; Askell, A.; Agarwal, S.; Herbert-Voss, A.; Krueger, G.; Henighan, T.; Child, R.; Ramesh, A.; Ziegler, D. M.; Wu, J.; Winter, C.; Hesse, C.; Chen, M.; Sigler, E.; Litwin, M.; Gray, S.; Chess, B.; Clark, J.; Berner, C.; McCandlish, S.; Radford, A.; Sutskever, I.; Amodei, D. Language Models are Few-Shot Learners. NeurIPS, 2020.
25. Kopka, M. L.; Yoon, C.; Goodsell, D.; Pjura, P.; Dickerson, R. E. The molecular origin of DNA-drug specificity in netropsin and distamycin. Proc. Nat. Acad. Sci. 1985, 82, 1376-1380.
26. Boger, D. L.; Fink, B. E.; Brunette, S. R.; Tse, W. C.; Hedrick, M. P. A Simple, High-Resolution Method for Establishing DNA Binding Affinity and Sequence Selectivity. J. Am. Chem. Soc. 2001, 123, 5878-5891.
27. Yao, H.; Jiang, F.; Lu, A.; Zhang, G. Methods to Design and Synthesize Antibody-drug Conjugates (ADCs). Int. J. Mol. Sci. 2016, 17, 194.
28. Lewis Phillips, G. D.; Li, G.; Dugger, D. L.; Crocker, L. M.; Parsons, K. L.; Mai, E.; Blättler, W A.; Lambert, J. M.; Chari, R. V. J.; Lutz, R. J.; Wong, W. L. T.; Jacobson, F. S.; Koeppen, H.; Schwall, R. H.; Kenkare-Mitra, S. R.; Spencer, S. D.; Sliwkowski, M. X. Targeting HER2-Positive Breast Cancer with Trastuzumab-DM1, an Antibody-Cytotoxic Drug Conjugate. Cancer Res. 2008, 68, 9280-9290.
29. Cheng, J.; Liang, M.; Carvalho, M. F.; Tigue, N.; Faggioni, R.; Roskos, L. K.; Vainshtein, I. Molecular Mechanism of HER2 Rapid Internalization and Redirected Trafficking Induced by Anti-HER2 Biparatopic Antibody. Antibodies 2020, 9, 49.
30. Lambert, J. M.; Chari, R. V. J. Ado-trastuzumab Emtansine (T-DM1): An Antibody-drug Conjugate (ADC) for Her2-positive Breast Cancer. J. Med. Chem. 2014, 57, 6949-6964.
31. Copolovici, D. M.; Langel, K.; Eriste, E.; Langel, U. Cell-Penetrating Peptides: Design, Synthesis, and Applications. ACS Nano 2014, 8, 1972-1994.
32. Pfaff, P.; Anderl, F.; Fink, M.; Balkenhohl, M.; Carreira, E., M. Azoacetylenes for the Synthesis of Arylazotriazole Photoswitches. Journal of the American Chemical Society. 2021, 143, 14495-14501.
33. Bremerich, M.; Conrads, C. M.; Langletz, T.; Bolm, C. Additions to N-Sulfinylamines as an Approach for the Metal-free Synthesis of Sulfonimidamides: O-Benzotriazolyl Sulfonimidates as Activated Intermediates. Angew. Chem., Int. Ed. 2019, 58, 19014-19020.
34. Heinrich, M. R.; Blank, O.; Ullrich, D.; Kirschstein, M. Allylation and Vinylation of Aryl Radicals Generated from Diazonium Salts. J. Org. Chem. 2007, 72, 9609-9616.
35. Tabey, A.; Berlande, M.; Hermange, P.; Fouquet, E. Mechanistic and asymmetric investigations of the Au-catalysed cross-coupling between aryldiazonium salts and arylboronic acids using (P,N) gold complexes. Chem. Commun. 2018, 54, 12867-12870.
36. Ma, X.; Herzon, S. B. Cobalt bis(acetylacetonate)-tert-butyl hydroperoxide-triethylsilane: a general reagent combination for the Markovnikov-selective hydrofunctionalization of alkenes by hydrogen atom transfer. Beilstein Journal of Organic Chemistry, 2018, 14, 2259-2265.
37. Effaty, F.; Gonnet, L.; Koenig, S. G.; Nagapudi, K.; Ottenwaelder, X.; Friščić, T. Resonant acoustic mixing (RAM) for efficient mechanoredox catalysis without grinding or impact media. Chemical Communications, 2023, 59, 1010-1013.
38. Burke, H. M.; Joullie, M. M. New synthetic routes to tilorone dihydrochloride and some of its analogs. Journal of Medicinal Chemistry, 1978, 21, 1084-1086.
39. Parveen, N.; Sekar, G. Palladium Nanoparticle-Catalyzed Stereoselective Domino Synthesis of 3-Allylidene-2(3H)-oxindoles and 3-Allylidene-2(3H)-benzofuranones. Journal of Organic Chemistry, 2020, 85, 4682-4694.
40. Nie, H.; Xiong, Z.; Hu, M.; Zhang, S.; Qin, C.; Wang, S.; Ji, F.; Jiang, G. Copper-Catalyzed Sulfonylation Reaction of NH-Sulfoximines with Aryldiazonium Tetrafluoroborates and Sulfur Dioxide: Formation of N-Sulfonyl Sulfoximines. Journal of Organic Chemistry, 2023, 88, 2322-2333.
41. Webb, E. W.; Cheng, K.; Wright, J. S.; Cha, J.; Shao, X.; Sanford, M. S.; Scott, P. J. H. Room-Temperature Copper-Mediated Radiocyanation of Aryldiazonium Salts and Aryl Iodides via Aryl Radical Intermediates. Journal of the American Chemical Society, 2023, 145, 6921-6926.
42. Marshall, N.; Locklin, J. Reductive electrografting of benzene (p-bisdiazonium hexafluorophosphate): a simple and effective protocol for creating diazonium-functionalized thin films. Langmuir, 2011, 27, 21, 13367-13373.
43. Ma, D.; Zhang, J.; Zhang, C.; Men, Y.; Sun, H.; Li, L-Y.; Yi, L.; Xi, Z. A highly efficient dual-diazonium reagent for protein crosslinking and construction of a virus-based gel. Organic & Biomolecular Chemistry, 2018, 16, 3353-3357.
44. Anuradha; Lathama, K.; Bhosale, S. V. Selective detection of nitrite ion by an AIE-active tetraphenylethene dye through a reduction step in aqueous media. RSC Advances, 2016, 6, 45009-45013.
45. Tolstaya, T. P.; Vanchikova, L. N.; Lisichkina, I. N. Bromination of triphenyloxonium cations. Russian Chemical Bulletin, 1984, 33, 1282-1285.
46. Bagal, I. L.; Luchkevich, E. R.; El'tsov, A. V. Transformations of Diazonium Salts. III. Transformations, Acid-Base Properties, and Reactivity of Nitronaphthyldiazonium Salts. Russian Journal of General Chemistry, 1996, 66, 119-132.
47. Russian Journal of Organic Chemistry USSR (English Translation), 1986, 914-920
48. Russian Journal of Organic Chemistry USSR (English Translation), 1967, 3, 392
49. Sengupta, S.; Sadhukhan, S. K. Fourfold Heck reactions on the tetraphenylmethane tripod: Model studies towards construction of centrally based three dimensional networks. Tetrahedron Letters, 1998, 39, 1237-1238.
50. Whiteker, G. T.; Lippard, S. J. Synthesis of thioether derivatives of ethidium bromide. Tetrahedron Letters, 1991, 32, 5019-5020
51. Damle, N. K.; Frost, P. Antibody-targeted chemotherapy with immunoconjugates of calicheamicin. Curr. Opin. Pharmacol. 2003, 3 (4), 386-390.
52. Hangeland, J. J.; De Voss, J. J.; Heath, J. A.; Townsend, C. A.; Ding, W. D.; Ashcroft, J. S.; Ellestad, G. A. Specific abstraction of the 5′S- and 4′-deoxyribosyl hydrogen atoms from DNA by calicheamicin γ₁^{I}. J. Am. Chem. Soc. 1992, 114 (23), 9200-9202.
53. Myers, A. G.; Cohen, S. B.; Kwon, B. M. A Study of the Reaction of Calicheamicin γ₁^{I} with Glutathione in the Presence of Double-Stranded DNA. J. Am. Chem. Soc. 1994, 116 (4), 1255-1271.
54. Kishikawa, H.; Jiang, Y. P.; Goodisman, J.; Dabrowiak, J. C. Coupled kinetic analysis of cleavage of DNA by esperamicin and calicheamicin. J. Am. Chem. Soc. 1991, 113 (14), 5434-5440.
55. Lee M. D.; Greenstein, M.; Labeda, D. P.; Fantini, A. A. Antitumor antibiotics (LL-E33288 complex), U.S. Pat. No. 5,108,912A, Apr. 28, 1992.
56. Chen, H.; Xiong, Z.; Zhang, A.; Ge, C.; Chang, F. Improving the Production of Antitumor Calicheamicin by the Micromonospora echinospora Mutant Coupled with in situ Resin Adsorption in Fermentation Process. Appl. Biochem. Microbiol. 2022, 58 (1), S132-S137.
57. Nguyen, T. D.; Bordeau, B. M.; Balthasar, J. P., Mechanisms of ADC Toxicity and Strategies to Increase ADC Tolerability. Cancers, 2023, 15 (3), 713.
58. McDonald, G. B.; Freston, J. W.; Boyer, J. L.; DeLeve, L. D. Liver Complications Following Treatment of Hematologic Malignancy With Anti-CD22-Calicheamicin (Inotuzumab Ozogamicin). Hepatology, 2019, 69 (2), 831-844.
59. Nicolaou, K. C.; Dai, W. M.; Tsay, S. C.; Estevez, V. A.; Wrasidlo, W. Designed Enediynes: A New Class of DNA-Cleaving Molecules with Potent and Selective Anticancer Activity. Science, 1992, 256, 1172-1178.
60. Kizil, M. Y.; Yilmaz, E. I.; Pirinccioglu, N.; and Aytekin, C. DNA Cleavage Activity of Diazonium Salts: Chemical Nucleases. Turk. J. Chem. 2003, 27 (5), 539-544.
61. Behr, J. P. Photohydrolysis of DNA by polyaminobenzenediazonium salts. J. Chem. Soc., Chem. Commun. 1989, (2), 101-103.
62. Starkey, E. B. p-Dinitrobenzene. Org. Synth. 1939, 19, 40.
63. Tse, W. C.; Boger, D. L. Sequence-Selective DNA Recognition: Natural Products and Nature's Lessons. Chem. Biol. 2004, 11 (12), 1607-1617.
64. Varakumar, P.; Rajagopal, K.; Aparna, B.; Raman, K.; Byran, G.; Gonçcalves Lima, C. M.; Rashid, S.; Nafady, M. H.; Emran, T. B.; Wybraniec, S. Acridine as an Anti-Tumour Agent: A Critical Review. Molecules, 2023, 28 (1), 193.
65. Ikemoto, N.; Kumar, R. A.; Ling, T. T.; Ellestad, G. A.; Danishefsky, S. J.; Patel, D. J. Calicheamicin-DNA complexes: warhead alignment and saccharide recognition of the minor groove. Proc. Natl. Acad. Sci. 1995, 92 (23), 10506-10510.
66. Yu, L.; Golik, J.; Harrison, R.; Dedon, P. The Deoxyfucose-Anthranilate of Esperamicin A1 Confers Intercalative DNA Binding and Causes a Switch in the Chemistry of Bistranded DNA Lesions. J. Am. Chem. Soc. 1994, 116 (21), 9733-9738.
67. Steenken, S.; Jovanovic, S. V. How Easily Oxidizable Is DNA? One-Electron Reduction Potentials of Adenosine and Guanosine Radicals in Aqueous Solution. J. Am. Chem. Soc. 1997, 119 (3), 617-618.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

1. A compound having the structure according to Formula (I): 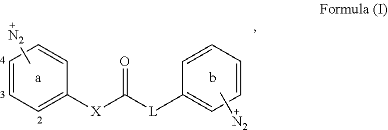
or a pharmaceutically acceptable salt thereof, wherein:
X is selected from the group consisting of S, Se, P, NH, and O; and
L is selected from the group consisting of S, Se, P, bond, and NH;
wherein:
when X is NH and L is bond, N₂⁺ in ring a is not located at the 4 position.

2. The compound of claim 1, wherein the compound is: 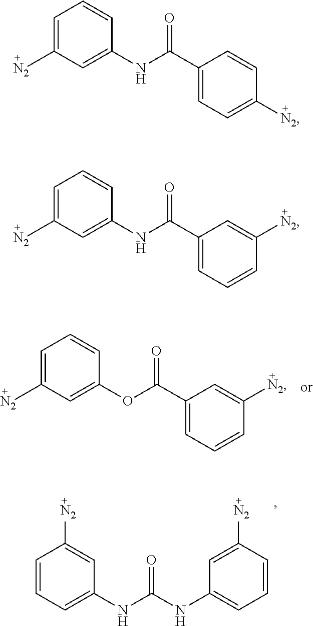
or a pharmaceutically acceptable salt thereof.

3.-11. (canceled)

12. A compound having the structure selected from the group consisting of Formula (II), Formula (III), Formula (IV), Formula (XVII), Formula (XVIII), Formula (XIX), Formula (XX), Formula (XXI), Formula (XXII), and Formula (XXIII), Formula (XXV), Formula (XXVI), Formula (XXVII), Formula (XXVIII), Formula (XXIX), Formula (XXX), and Formula (XXXI): 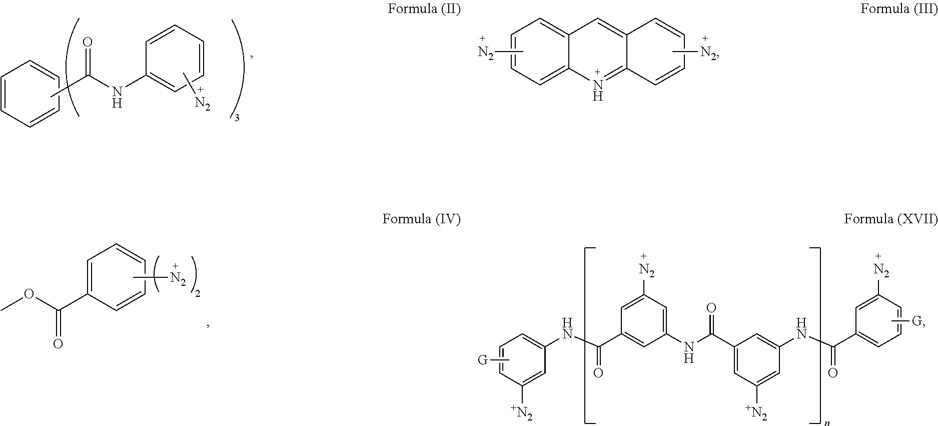 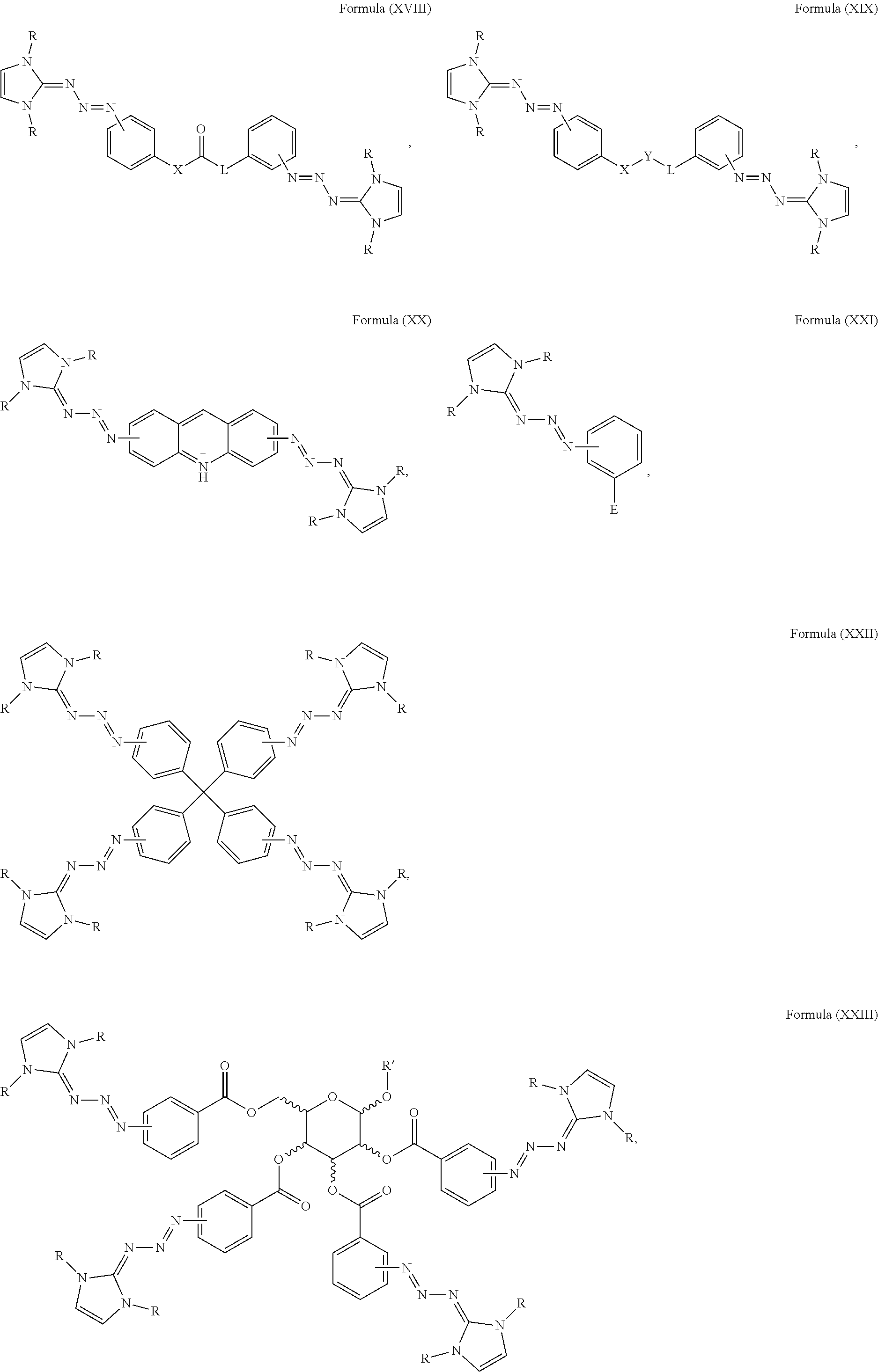 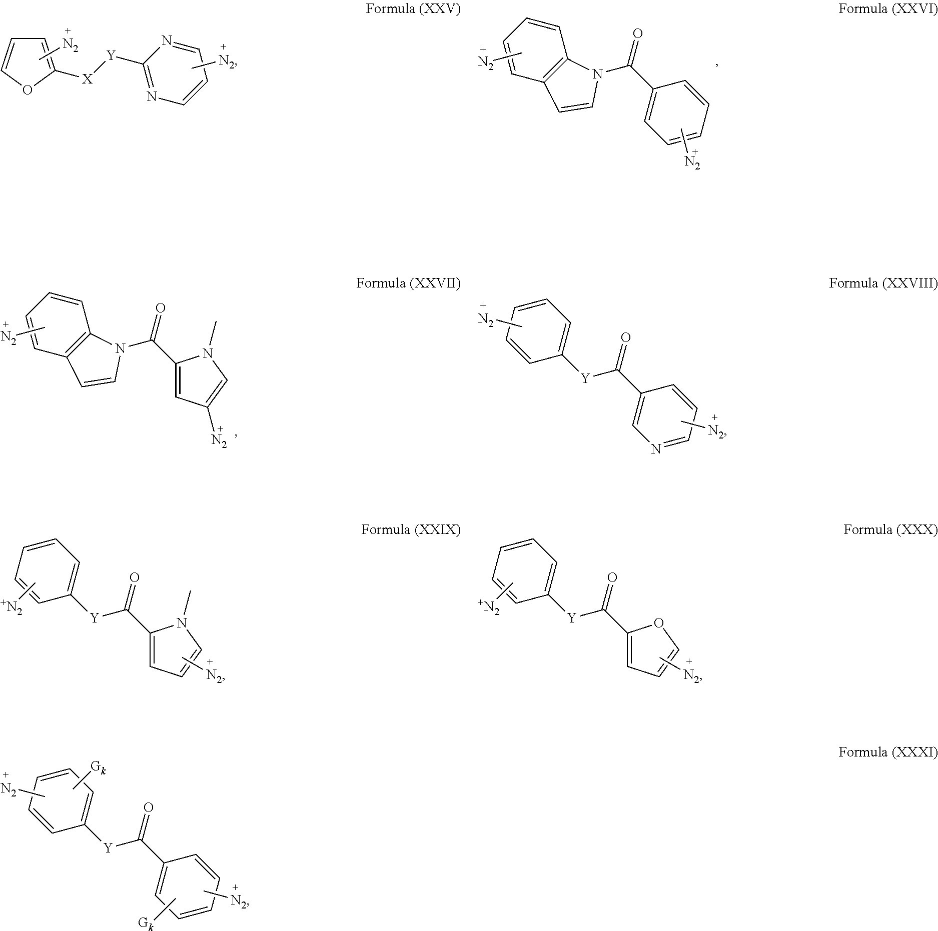
or a pharmaceutically acceptable salt thereof, wherein:
X and Y are independently selected from the group consisting of bond, alkyl, S, Se, P, C═O, NH, NR′, O, amide, and ester;
k is independently at each occurrence from 0 to 4;
G is independently at each occurrence selected from the group consisting of H, halogen, carbonyl, ester, ketone, amide, alkyl, cycloalkyl, aryl, arylalkyl, alkenyl, heteroalkyl, cyano, alkoxy, amine, haloalkyl, heteroatoms, and heterocycle;
E is selected from the group consisting of CN, CO₂Me, OMe, and NO₂;
L is selected from the group consisting of S, Se, P, bond, and NH;
n is selected from the group consisting of 1, 2, 3, 4, 5, 6, 7, 8, 9, and 10;
R is selected from the group consisting of methyl, ethyl, isopropyl, mesityl, and alkyl sulfonate; and
R′ is selected from the group consisting of H, alkyl, cycloalkyl, halogen, alkenyl, aryl, arylalkyl, haloalkyl, heteroalkyl, acetyl, heterocycle, amine, amide, ether, and carbonyl, and any combination thereof.

13. A compound having the structure selected from the group consisting of Formula (I), Formula (II), Formula (III), Formula (IV), Formula (VI), Formula (VII), Formula (VIII), Formula (IX), Formula (X), Formula (XI), Formula (XII), Formula (XVII), Formula (XVIII), Formula (XIX), Formula (XX), Formula (XXI), Formula (XXII), Formula (XXIII), Formula (XXIV), Formula (XXV), Formula (XXVI), Formula (XXVII), Formula (XXVIII), Formula (XXIX), Formula (XXX), Formula (XXXI), and Formula (XXXII): 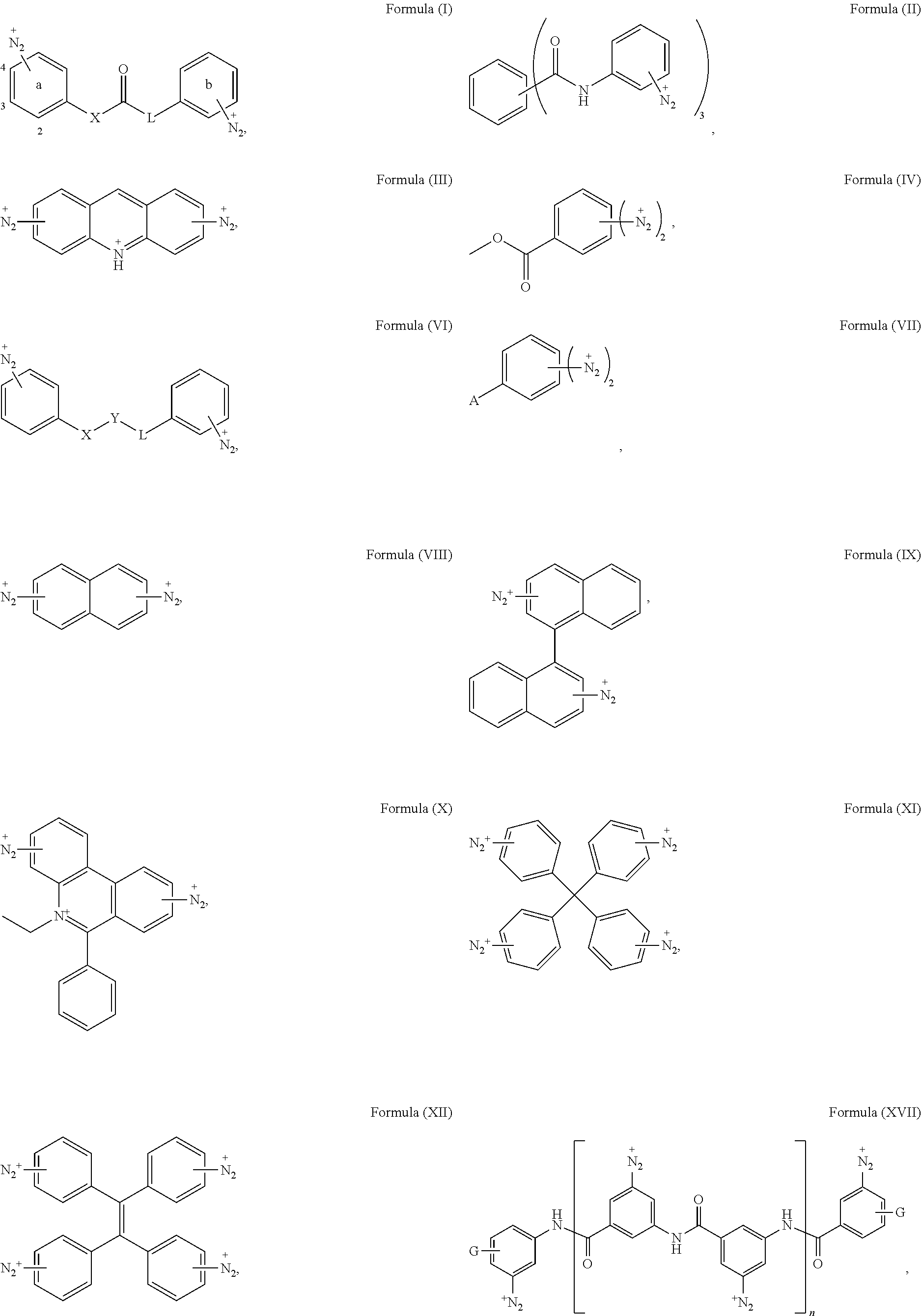 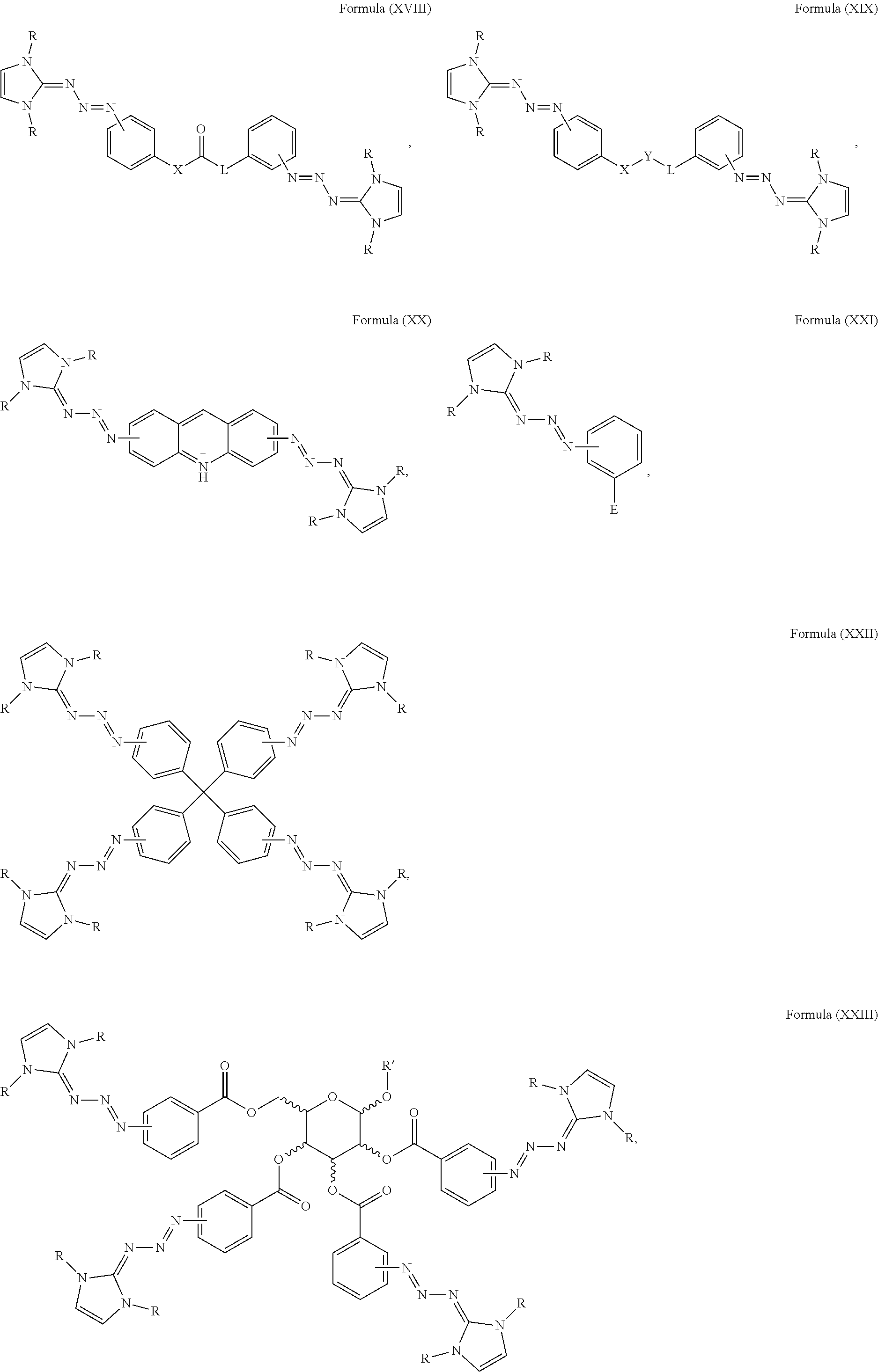 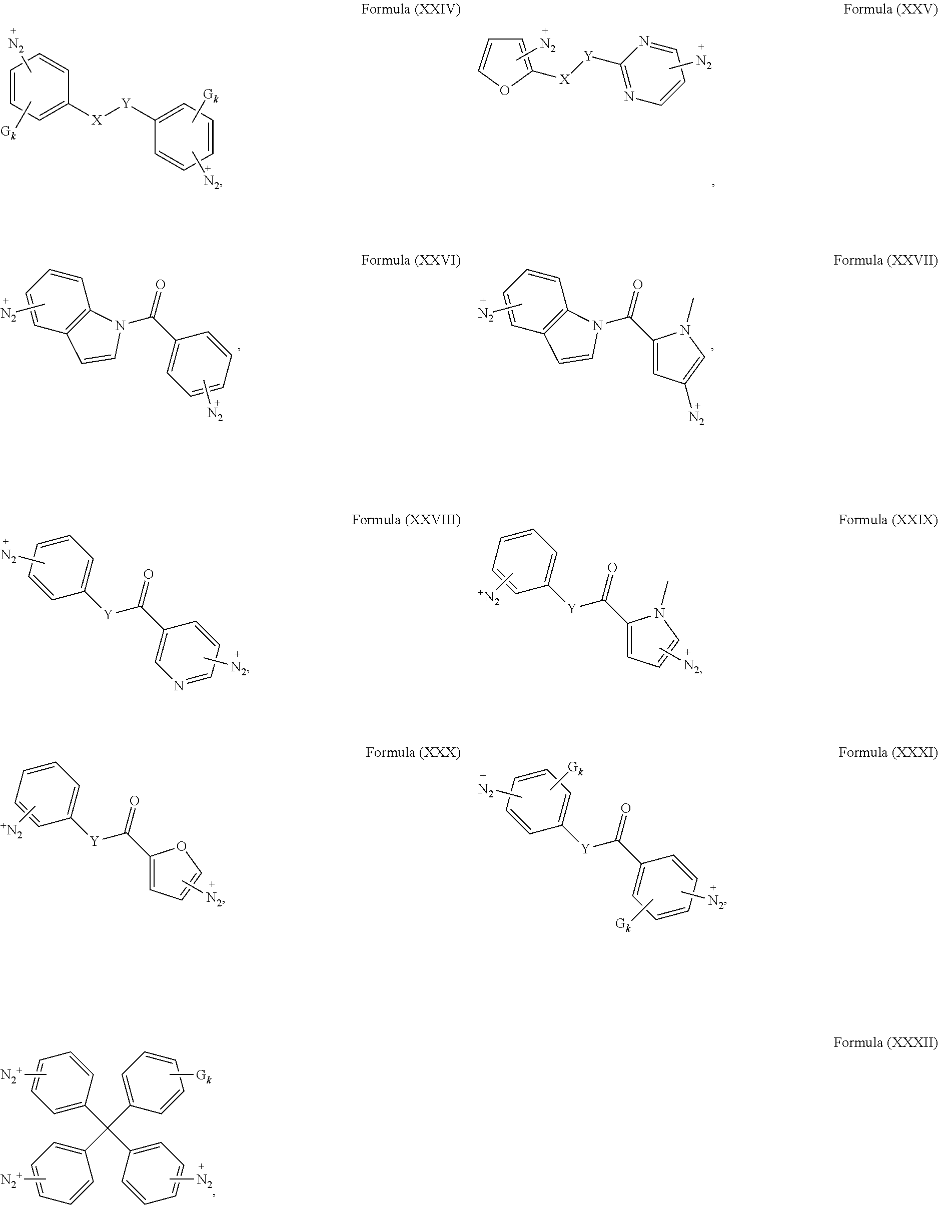
or a pharmaceutically acceptable salt thereof, wherein:
X is selected from the group consisting of S, Se, P, bond, alkyl, C═O, NH, NR′, amide, ester, and O;
L is selected from the group consisting of S, Se, P, bond, and NH;
Y is selected from the group consisting of bond, alkyl, S, Se, P, NH, NR′, amide, ester, O, and C═O;
A is selected from the group consisting of CO₂Me and H;
E is selected from the group consisting of CN, CO₂Me, OMe, and NO₂;
G is independently at each occurrence selected from the group consisting of H, halogen, carbonyl, ester, ketone, amide, alkyl, cycloalkyl, aryl, arylalkyl, alkenyl, heteroalkyl, cyano, alkoxy, amine, haloalkyl, heteroatoms, and heterocycle;
n is selected from the group consisting of 1, 2, 3, 4, 5, 6, 7, 8, 9, and 10;
k is independently at each occurrence from 0 to 4;
R is selected from the group consisting of methyl, ethyl, isopropyl, mesityl, and alkyl sulfonate; and
R′ is selected from the group consisting of H, alkyl, cycloalkyl, halogen, alkenyl, aryl, arylalkyl, haloalkyl, heteroalkyl, acetyl, heterocycle, amine, amide, ether, and carbonyl, and any combination thereof, wherein said compound, optionally in addition to more than one molecule of said compound, is conjugated to a compound selected from the group consisting of a sugar, a heterocycle, an antibody, a minor groove binder, an intercalator, an amino acid, and a peptide.

14. (canceled)

15. The compound of claim 13, wherein the conjugation is via a linker selected from the group consisting of alkyl, heteroatom, heteroalkyl, haloalkyl, alkenyl, alkoxy, ester, ether, amide, hydrazine, hydrazone, acetal, ketal, disulfide, phosphate, Mal-PEG-NHS ester, DBCO-NHS ester, and SMCC.

16.-17. (canceled)

18. The compound of claim 13, wherein the compound has the structure selected from the group consisting of Formula (XV) and Formula (XVI): 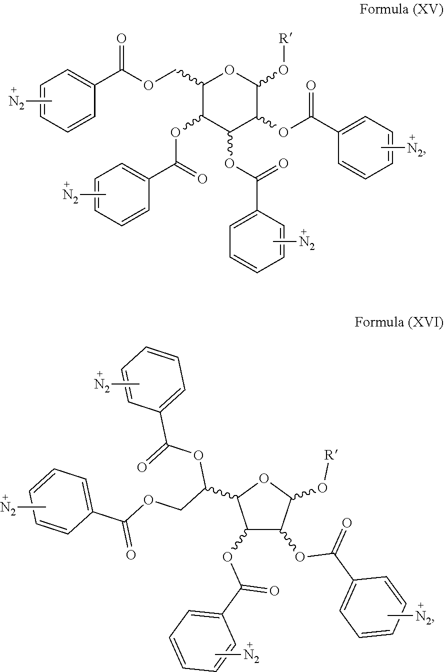
or a pharmaceutically acceptable salt thereof, wherein:
R′ is selected from the group consisting of H, alkyl, cycloalkyl, halogen, alkenyl, aryl, arylalkyl, haloalkyl, heteroalkyl, acetyl, heterocycle, amine, amide, ether, carbonyl, maleimide (Mal)-polyethylene glycol (PEG)-N-Hydroxysuccinimide (NHS) ester, dibenzocyclooctyne (DBCO)—NHS ester, and succinimidyl 4-(N-maleimidomethyl)cyclohexane-1-carboxylate (SMCC), and any combination thereof.

19. The compound of claim 18, wherein the compound is: 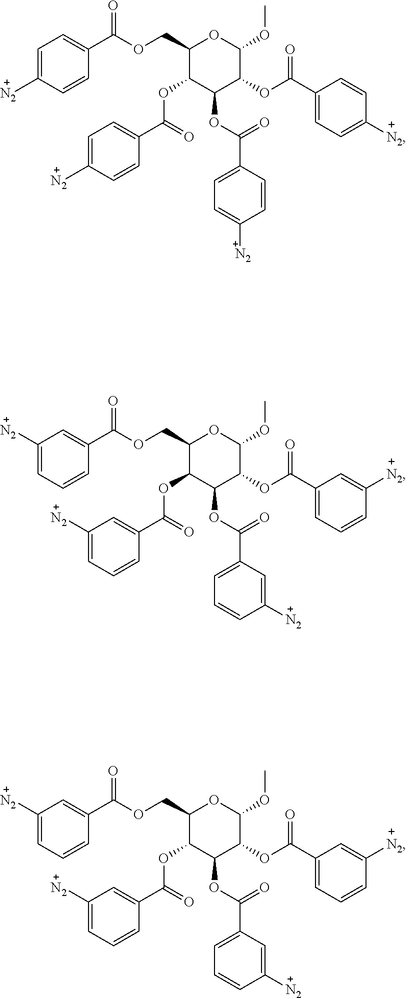
or a pharmaceutically acceptable salt thereof.

20. The compound of claim 13, wherein the sugar is glucose, galactose, mannose, or fructose.

21.-22. (canceled)

23. The compound of claim 13, wherein the sugar is a monosaccharide, a disaccharide, or a polysaccharide.

24.-25. (canceled)

26. The compound of claim 13, wherein the compound is conjugated to a group selected from the group consisting of a heterocycle, an antibody, a minor groove binder, an intercalator, an amino acid, and a peptide.

27.-36. (canceled)

37. The pharmaceutical composition comprising the compound of claim 1, or a pharmaceutically acceptable salt thereof.

38. The pharmaceutical dosage form comprising the compound of claim 1 or a pharmaceutically acceptable salt thereof.

39. A method of cleaving DNA in a cell comprising contacting the cell with an effective amount of the compound of claim 1.

40. A method of cleaving DNA in a cell comprising contacting the cell with an effective amount of the compound of claim 13, wherein the compound is according to Formula (VI), Formula (VII), Formula (VIII), Formula (IX), Formula (X), Formula (XI), Formula (XII), Formula (XXIV), or Formula (XXXII): 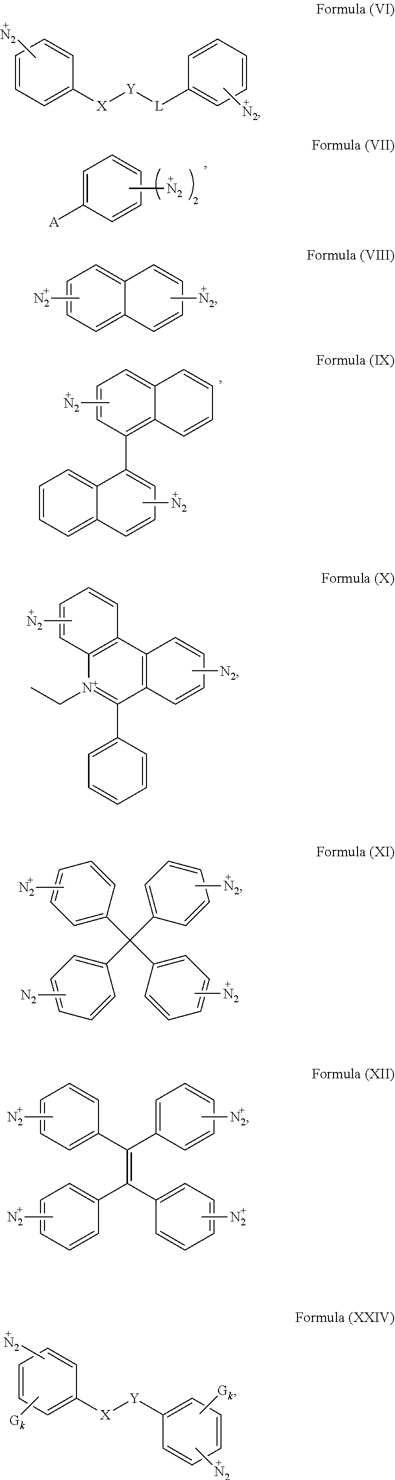 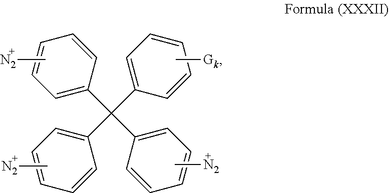
or a pharmaceutically acceptable sale thereof, wherein:
X and Y are independently selected from the group consisting of bond, alkyl, S, Se, P, C═O, NH, NR′, S, O, amide, and ester;
k is independently at each occurrence from 0 to 4;
G is independently at each occurrence selected from the group consisting of H, halogen, carbonyl, ester, ketone, amide, alkyl, cycloalkyl, aryl, arylalkyl, alkenyl, heteroalkyl, cyano, alkoxy, amine, haloalkyl, heteroatoms, and heterocycle;
A is selected from the group consisting of CO₂Me and H;
L is selected from the group consisting of S, Se, P, bond, and NH; and
R′ is selected from the group consisting of H, alkyl, cycloalkyl, halogen, alkenyl, aryl, arylalkyl, haloalkyl, heteroalkyl, acetyl, heterocycle, amine, amide, ether, and carbonyl, and any combination thereof.

41. (canceled)

42. The method of claim 40, wherein the compound is: 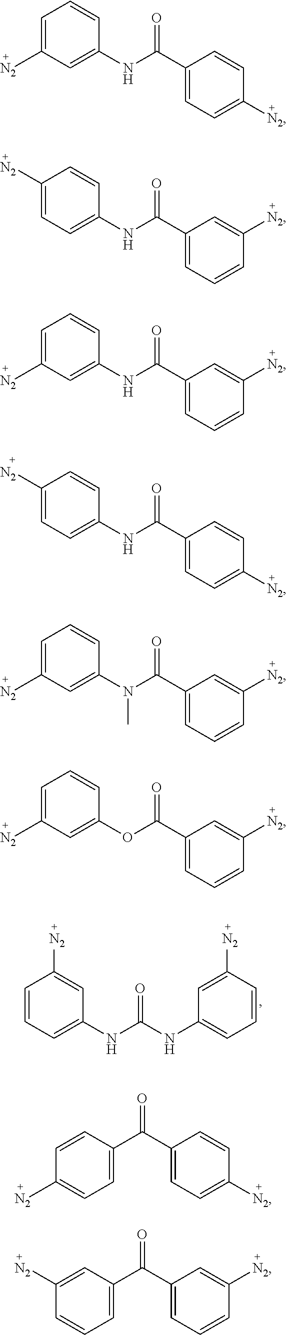 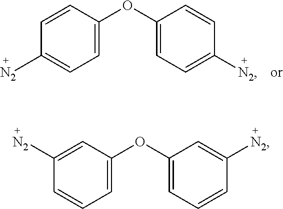
or a pharmaceutically acceptable salt thereof.

43.-45. (canceled)

46. The method of claim 40, wherein the compound is: 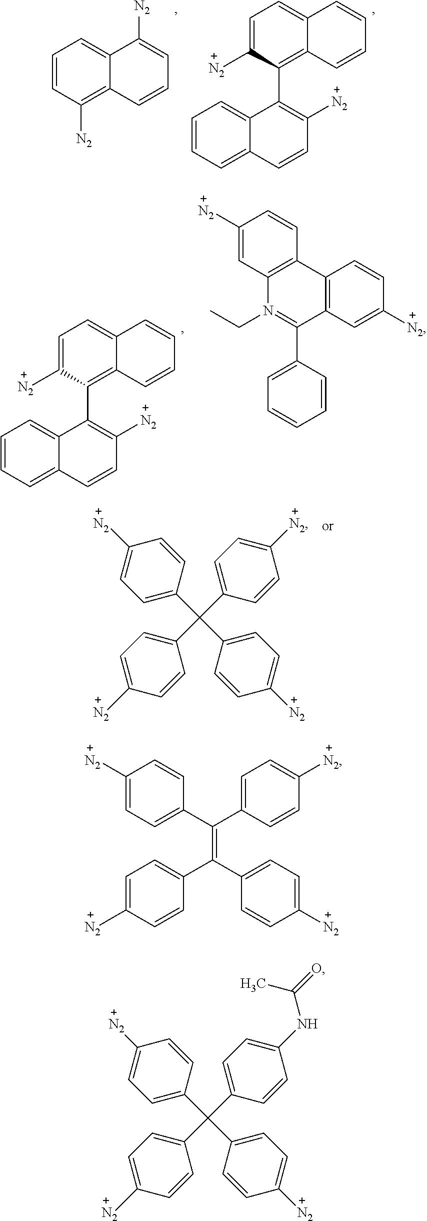
or a pharmaceutically acceptable salt thereof.

47.-51. (canceled)

52. The method of claim 39, wherein the method further comprises a reduction of the compound to its corresponding radical upon nitrogen extrusion after contacting the cell with the compound.

53. (canceled)

54. The method of claim 52, wherein the reduction comprises light irradiation performed with light of a wavelength comprising one or more wavelengths within the full spectrum of light wavelengths.

55.-79. (canceled)

80. A method of treating a bacterial infection, a viral infection, or a cancer in a subject in need thereof, comprising administering to the subject an effective amount of the compound of claim 1.

81.-86. (canceled)

87. The method of claim 80, wherein the cancer is selected from the group consisting of kidney cancer, adrenal gland cancer, anal cancer, appendiceal cancer, ovarian cancer, uterine cancer, gastric cancer, breast cancer, lung cancer, bladder cancer, cervical cancer, stomach cancer, sarcoma cancer, liver cancer, esophageal cancer, laryngeal cancer, multiple myeloma, colorectal cancer, rectal cancer, skin cancer, pancreatic cancer, brain or spinal cord cancer, leukemia or lymphoma.

88.-90. (canceled)
